(19) United States
(12) Patent Application Publication
(10) Pub. No.: US 2010/0029615 A1
(43) Pub. Date: Feb. 4, 2010

(54) BENZIMIDAZOLE DERIVATIVES

(76) Inventors: Michael John Munchhof, Salem, CT (US); Lawrence Alan Reiter, Mystic, CT (US); Andrei Shavnya, East Lyme, CT (US); Christopher Scott Jones, Gales Ferry, CT (US); Qifang Li, Stonington, CT (US); Robert Gerald Linde ll, Old Lyme, CT (US)

Correspondence Address: PFIZER INC, 10555 SCIENCE CENTER DRIVE, SAN DIEGO, CA, 92121, US

(21) Appl. No.: 12/518,970

(22) PCT Filed: Dec. 5, 2007
(86) PCT No.: PCT/IB07/04144

§ 371 Date: Jun. 12, 2009

Related U.S. Application Data
(60) Provisional application No. 60/870,360, filed on Dec. 15, 2006.
(60) Provisional application No. 60/887,626, filed on Feb. 1, 2007.

(51) Int. Cl. A61K 31/55; A61K 31/538; A61K 31/4709; A61K 31/4178; C07D 403/12; C07D 413/12; C07D 401/12

(52) U.S. Cl. 514/212.08; 514/230.5; 514/314; 514/406; 540/524; 544/105; 546/166; 548/305.1

(57) ABSTRACT

The present invention relates to a compound of the formula (I) or a pharmaceutically acceptable salt thereof, wherein R^{1,} R², R³, R⁴, R⁵, A, X, n, and are as defined herein. Such novel benzamidazole derivatives are useful in trv treatment of abnormal cell growth, such as cancer, in mammals. This invention ate relates to a method of using such compounds in the treatment of abnormal cell growth in mammals, especially humans, and to pharmaceutical compositions containing sue compounds.

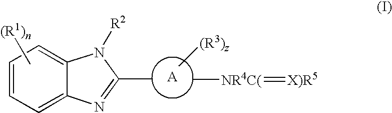

This application claims the benefit of U.S. Application No. 60/870,360, filed Dec. 15, 2006, and U.S. Application No. 60/887,626, filed Feb. 1, 2007, both of which are hereby incorporated by reference in their entirety.

### FIELD OF INVENTION

This invention relates to novel benzimidazole derivatives that are useful in the treatment of abnormal cell growth, such as cancer, in mammals. This invention also relates to a method of using such compounds in the treatment of abnormal cell growth in mammals, especially humans, and to pharmaceutical compositions containing such compounds.

### BACKGROUND OF THE INVENTION

Hedgehog (Hh) proteins are secreted morphogens that are involved in many biological processes during embryonic development. Postnatally, Hh has important roles in tissue homeostasis and aberrant Hh signaling is associated with developmental disorders and several types of cancer. At the cell surface, the Hh signal is thought to be relayed by the 12 transmembrane domain protein Patched (Ptc) (Hooper and Scott, Cell 59: 75 1-65 (1989); Nakano et al., Nature 341: 508-13 (1989)) and the G-protein-coupled-like receptor Smoothened (Smo) (Alcedo et al., Cell 86: 221-232 (1996); van den Heuvel and Tngham, Nature 382: 547-551 (1996)). Both genetic and biochemical evidence support a receptor model where Ptch and Smo are part of a multi-component receptor complex (Chen and Struhl, Cell 87: 553-63 (1996); Mango et al., Nature 384: 176-9 (1996); Stone et al., Nature 384:129-34 (1996)). Upon binding of Hh to Ptch, the normal inhibitory effect of Ptch on Smo is relieved, allowing Smo to transduce the Hh signal across the plasma membrane. However, the exact mechanism by which Ptch controls Smo activity still has yet to be clarified.

The signaling cascade initiated by Smo results in activation of Gli transcription factors that translocate into the nucleus where they control transcription of target genes. Gli has been shown to influence transcription of Hh pathway inhibitors such as Ptc and Hip I in a negative feedback loop indicating that tight control of the Hh pathway activity is required for proper cellular differentiation and organ formation. Uncontrolled activation of Hh signaling pathway is associated with malignancies in particular those of the brain, skin and muscle as well as angiogenesis. An explanation for this is that the Hh pathway has been shown to regulate cell proliferation in adults by activation of genes involved in cell cycle progression such as cyclin D which is involved in G1-S transition. Also, Sonic Hedgehog (SHh), an ortholog of Hh, blocks cell-cycle arrest mediated by p21, an inhibitor of cyclin dependent kinases. Hh signaling is further implicated in cancer by inducing components in the EGFR pathway (EGF, Her2) involved in proliferation as well as components in the PDGF (PDGFa) and VEGF pathways involved in angiogenesis. Loss of function mutations in the Ptch gene have been identified in patients with the basal cell nevus syndrome (BCNS), a hereditary disease characterized by multiple basal cell carcinomas (BCCs). Dysfunctional Ptch gene mutations have also been associated with a large percentage of sporadic basal cell carcinoma tumors (Chidambaram et al., Cancer Research 56: 4599-601 (1996); Gailani et al., Nature Genet. 14: 78-81 (1996); Hahn et al., Cell 85: 841-51 (1996); Johnson et al., Science 272: 1668-71 (1996); Unden et al., Cancer Res. 56: 4562-5; Wicking et al., Am. J. Hum. Genet. 60: 21-6 (1997)). Loss of Ptch function is thought to cause an uncontrolled Smo signaling in basal cell carcinoma. Similarly, activating Smo mutations have been identified in sporadic BCC tumors (Xie et al., Nature 391: 90-2 (1998)), emphasizing the role of Smo as the signaling subunit in the receptor complex for SHh. Various inhibitors of hedgehog signaling have been investigated such as Cyclopamine, a natural alkaloid that has been shown to arrest cell cycle at G0-GI and to induce apoptosis in SCLC. Cyclopamine is believed to inhibit Smo by binding to its heptahelical bundle. Forskolin has been shown to inhibit the Hh pathway downstream from Smo by activating protein kinase A (PKA) which maintains Gli transcription factors inactive. Despite advances with these and other compounds, there remains a need for potent inhibitors of the hedgehog signaling pathway.

### SUMMARY OF THE INVENTION

The present invention relates to a compound of formula I:

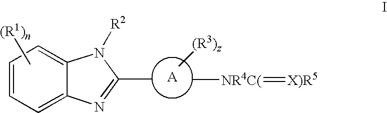

or a pharmaceutically acceptable salt wherein:

A is a 1,3-(C₃-C₁₂)cycloalkyl;

each R¹ is independently selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜ(C═O)N[(CH₂)ₜR⁹]₂, —(CH₂)ₜN[(CH₂)ₜR⁹](C═O)[(CH₂)ₜR⁹], —(CH₂)ₜN[(CH₂)ₜR⁹]S(O)_{w}[(CH₂)ₜR⁹], —(CH₂)ₜS(O)_{w}N[(CH₂)ₜR⁹]₂, —(CH₂)ₜS(O)_{w}[(CH₂)ₜR⁹], —(CH₂)ₜR⁹, —(CH₂)ₜO[(CH₂)ₜR⁹], —(CH₂)ₜ(C═O )[(CH₂)ₜR⁹]₂, —(CH₂)ₜ(C═O)O((CH₂)ₜR⁹], —(CH₂)ₜO(C═O)[(CH₂)ₜR⁹], —N[(C H₂)ₜR⁹](C═O)N[(CH₂)ₜR⁹]₂, —(CH₂)ₜ(C₃-C₁₂)carbocyclyl, —(CH₂)ₜ(C₆-C₁₀ aryl), and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂;

R² is selected from the group consisting of hydrogen, —(C₁-C₆)alkyl, —(CH₂)_{q}OH, —(CH₂)_{q}O(C₁-C₆)alkyl, —(CH₂)_{q}O(C₁-C₆)alkylOH, —(CH₂)ₚCF₃, and —(CH₂)ₚCN;

each R³ is independently selected from the group consisting of hydrogen, —CN, halo, hydroxy, —(C₁-C₆)alkyl, —(C₂-C₆)alkenyl, —(C₂-C₆)alkynyl, —(C₁-C₆)alkoxy, —CF₃, —OCF₃, —N[(CH₂)ₜR⁹]₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, —(C₃-C₁₂)carbocyclyl, —(CH₂)ₜ(C₆-C₁₀ aryl), —(CH₂)ₜ(4 to 14 membered heterocyclyl), —(CH₂)ₜO(CH₂)ₜ(C₆-C₁₀ aryl), —(CH₂)ₜO(CH₂)ₜ(4 to 14 membered heterocyclyl), —(CH₂)ₜ(C═O)(CH₂)ₜ(C₆-C₁₀ aryl), —(CH₂)ₜ(C═O)(CH₂)ₜ(4 to 14 membered heterocyclyl), wherein said heterocyclyl has 1 to 4 ring heteroatoms selected from the group consisting of N, O, and S, and wherein each said alkyl, cycloalkyl, aryl, and heterocyclyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl;

R⁴ is selected from the group consisting of hydrogen, —(C₁-C₆)alkyl, —(CH₂)_{q}OH, —(CH₂)_{q}O(C₁-C₆)alkyl, —(CH₂)_{q}O(C₁-C₆)alkylOH, —(CH₂)ₚCF₃, —(CH₂)ₚCN, —(CH₂)ₚNH₂—(CH₂)ₚNH(C₁-C₆)alkyl, and —(CH₂)ₚN[(C₁-C₆)alkyl]₂;

R⁵ is selected from the group consisting of —(C₁-C₆)alkyl, —(C₂-C₆)alkenyl, —(C₂-C₆)alkyny), —(CH₂)ₜ(C₃-C₁₂)carbocyclyl, —(CH₂)ₜ(C₆-C₁₀)aryl, —(CH₂)ₚ(C₁-C₆)alkoxy, —(CH₂)ₜO(C H₂)ₜ(C₆-C₁₀)aryl, —(CH₂)ₜN[(CH₂)ₜR⁹]₂, —(CH₂)ₜN[(CH₂)ₜR⁹](C₆-C₁₀)aryl, —(CH₂)ₜ(4 to 14 membered heterocyclyl), —(CH₂)ₜO(CH₂)ₜ(4 to 14 membered heterocyclyl) and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, and wherein each said (CH₂) moiety, alkyl, alkynyl, alkenyl, carbocyclyl, aryl, and heterocyclyl are independently optionally substituted by 1 to 5 substituents selected from R⁶;

each R⁶ is independently selected from the group consisting of azide, halo, —NO₂, —OR⁷, —(CH₂)ₜ(R⁷), —CF₃, —OCF₃, —OCHF₂, —OCH₂F, —O(CH₂)ₜ(C₆-C₁₀)aryl(R⁷), —(CH₂)ₜ(C≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜSR⁷, —(CH₂)ₜ(S═O)R⁷, —(CH₂)ₜS(═O)₂R⁷, —[C(R⁶)₂]ₜN(R⁷)S(═O)₂R⁷, —S(═O)₂N(R⁷)₂, —(C═O)R⁷, —(C═O)OR⁷, —[C(R⁷)₂]ₜO(C═O)R⁷, —[C(R⁷)₂]ₜO(C═O)N(R⁷)₂, —[C(R⁷)₂]ₜN(R⁷)(C═O)R⁷, —[C(R⁷)₂]₂N(R⁷)₂, —[C(R⁷)₂]ₜOR⁷, —[C(R⁷)₂]ₜN(R⁷)(C═O)OR⁷, —[C(R⁷)₂]ₜN(R⁷)(C═O)N(R⁷)₂, —[C(R⁷)₂]ₜM(R⁷)S(═O)₂N(R⁷)₂, —[C(R⁷)₂]ₜN(R⁷)N(R⁷)₂, —(C═O)N(R⁷)₂, —O(C═O)N(R⁷)₂, —[C(R⁷)₂]ₜOR⁷, —C(R⁷)₂SR⁷, —[C(R⁷)₂]ₜ(S═O)R⁷, —[C(R⁷)₂]ₜS(═O)₂R⁷, —C(R⁷)₂]ₜS(═O)₂N(R⁷)₂, —[C(R⁷)₂]ₜN(R⁷)(C═O)R⁷, —[C(R⁷)₂]ₜN(R⁷)(C═O)OR⁷, —C(R⁷)═NN(R⁷)₂, —C(R⁷)═NOR⁷, —C(R⁷)₂N(R⁷)N(R⁷)₂, —[C(R⁷)₂]ₜN(R⁷)S(═O)₂N(R⁷)₂, and —[C(R⁷)₂]ₜN(R⁷)(C═O)N(R⁷)₂;

each R⁷ is independently selected from H, —CF₃, —(C₁-C₆)alkyl, —(C₂-C₆)alkenyl, —(C₂-C₆)alkynyl, —(C₃-C₁₂)carbocyclyl, and —(C₆-C₁₀)aryl, or two R⁷ groups on the same nitrogen atom may be taken together with the nitrogen atom to form a 5 to 8 membered heterocyclyl ring, wherein said heterocyclyl ring has 1 to 3 ring heteroatoms selected from the group consisting of N, O, and S, or two R⁷ groups on the same carbon atom may be taken together with the carbon atom to form a 3 to 7 membered carbocyclyl ring and wherein each said alkyl, alkenyl, aryl, heterocyclyl and carbocyclyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₅)alkyl, —(C═O)O(C₁-C₆)alkyl, —C(═O)(C₁-C₆)alkyl, and —(C₃-C₁₂)carbocyclyl;

X is selected from the group consisting of O, S, and NR³;

R⁸ is selected from the group consisting of hydrogen, —(C₁-C₆)alkyl, —(CH₂)ₜC≡N, —NO₂, and —S(═O)₂R⁹;

each R⁹ is independently selected from the group consisting of H, —(C₁-C₆)alkyl, —(CH₂)ₜOH, —(CH₂)ₜ(C₆-C₁₀ aryl), —(CH₂)ₜ(C₃-C₁₂)carbocyclyl, and —(CH₂)ₜ(4 to 14 membered heterocyclyl), wherein said heterocyclyl has 1 to 3 ring heteroatoms selected from the group consisting of N, O, and S, or two R⁹ groups on the same nitrogen atom may be taken together with the nitrogen atom to form a 5 to 8 membered heterocyclyl ring wherein said heterocyclyl ring optionally has 1 to 3 ring additional heteroatoms selected from the group consisting of N, O, and S, or two R⁹ groups on the same carbon atom may be taken together with the carbon atom to form a 3 to 7 membered carbocyclyl ring, wherein each said alkyl, aryl, (CH₂) moiety, carbocyclyl, and heterocyclyl may optionally be substituted by one to three substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S;

each p is an integer independently selected from 1, 2, 3, 4, or 5;

each t is an integer independently selected from 0, 1, 2, 3, 4, or 5;

each n is an integer independently selected from 0, 1, 2, 3, or 4;

each q is an integer independently selected from 2, 3, 4, or 5;

each w is an integer independently selected from 0, 1, or 2; and

each z is an integer independently selected from 0, 1, 2, 3, 4, 5, 6, or 7.

In one embodiment the invention relates to a compound of Formula I or pharmaceutically acceptable salt thereof, wherein A is a 1,3-(C₃-C₉)cycloalkyl.

In one embodiment the invention relates to a compound of Formula I or pharmaceutically acceptable salt thereof, wherein A is a 1,3-(C₄-C₆)cycloalkyl.

In one embodiment the invention relates to a compound of Formula I or pharmaceutically acceptable salt thereof, wherein A is a 1,3-(C₃)cycloalkyl.

In one embodiment the invention relates to a compound of Formula I or pharmaceutically acceptable salt thereof, wherein A is a 1,3-(C₄)cycloalkyl.

In a preferred embodiment the invention relates to a compound of Formula I or pharmaceutically acceptable salt thereof, wherein A is a 1,3-cyclobutyl.

In one embodiment the invention relates to a compound of Formula I or pharmaceutically acceptable salt thereof, wherein A is a 1,3-(C₅)cycloalkyl.

In one embodiment the invention relates to a compound of Formula I or pharmaceutically acceptable salt thereof, wherein A is a 1,3-(C₆)cycloalkyl.

In a more preferred embodiment the invention relates to a compound of Formula I or pharmaceutically acceptable salt thereof, wherein A is a 1,3-cyclohexyl.

In one embodiment the invention relates to a compound of Formula I or pharmaceutically acceptable salt thereof, wherein A is a 1,3-(C₇)cycloalkyl.

In one embodiment the invention relates to a compound of Formula I or pharmaceutically acceptable salt thereof, wherein A is a 1,3-(C₈)cycloalkyl.

In one embodiment the invention relates to a compound of Formula I or pharmaceutically acceptable salt thereof, wherein A is a 1,3-(C₉)cycloalkyl.

In one embodiment the invention relates to a compound of Formula I or pharmaceutically acceptable salt thereof, wherein A is a 1,3-(C₁₀)cycloalkyl.

In one embodiment the invention relates to a compound of Formula I or pharmaceutically acceptable salt thereof, wherein A is a 1,3-(C₁₁)cycloalkyl.

In one embodiment the invention relates to a compound of Formula I or pharmaceutically acceptable salt thereof, wherein A is a 1,3-(C₁₂)cycloalkyl.

In another embodiment the invention relates to a compound of Formula I or pharmaceutically acceptable salt thereof, wherein A is a bicyclo[3.1.1]heptanyl, said ring may optionally contain 1 or 2 double bonds.

In another embodiment the invention relates to a compound of Formula I or pharmaceutically acceptable salt thereof, wherein A is a bicyclo[2.2.1]heptanyl, said ring may optionally contain 1 or 2 double bonds.

In another embodiment the invention relates to a compound of Formula I or pharmaceutically acceptable salt thereof, wherein A is a bicyclo[3.2.1]octanyl, said ring may optionally contain 1 or 2 double bonds.

In another embodiment the invention relates to a compound of Formula I or pharmaceutically acceptable salt thereof, wherein A is a bicyclo[5.2.0]nonanyl, said ring may optionally contain 1 or 2 double bonds.

In another embodiment the invention relates to a compound of Formula I or pharmaceutically acceptable salt thereof, wherein A is a norbornyl, said ring may optionally contain 1 or 2 double bonds.

In another embodiment the invention relates to a compound of Formula I or pharmaceutically acceptable salt thereof, wherein A is an adamantanyl, said ring may optionally contain 1 or 2 double bonds.

In another embodiment the invention relates to a compound of Formula I or pharmaceutically acceptable salt thereof, wherein A is a spiro cycloalkyl.

In a preferred embodiment the invention relates to a compound of Formula I or pharmaceutically acceptable salt thereof, wherein the 3 position has absolute configuration R.

In a more preferred embodiment the invention relates to a compound of Formula I or pharmaceutically acceptable salt thereof, wherein the 3 position has absolute configuration R and the 1 position has absolute configuration S.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜ(C═O )N[(CH₂)ₜR⁹]₂, —(CH₂)ₜN[(CH₂)ₜR⁹](C═O)[(CH₂)ₜR⁹], —(CH₂)ₜN[(CH₂)ₜR⁹]S(O)_{w}[(CH₂)ₜR⁹], —(CH₂)ₜR⁹, —(CH₂)ₜO[(CH₂)ₜR⁹], —(CH₂)ₜ(C═O)[(CH₂)ₜR⁹], —(CH₂)ₜ(C═O)O[(CH₂)ₜR⁹], —(CH₂)ₜO(C═O)[(CH₂)ₜR⁹], —N[(CH₂)ₜR⁹](C═O)N[(CH₂)ₜR⁹]₂, —(CH₂)ₜ(C₃-C₁₂)carbocyclyl, —(CH₂)ₜ(C₆-C₁₀ aryl), and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜ(C═O)N[(CH₂)ₜR⁹]₂, —(CH₂)ₜN[(CH₂)ₜR⁹](C═O)[(CH₂)ₜR⁹], —(CH₂)ₜN[(CH₂)ₜR⁹]S(O)_{w}[(CH₂)ₜR⁹], —(CH₂)ₜR⁹, —(CH₂)ₜO[(CH₂)ₜR⁹], —(CH₂)ₜ(C═O)[(CH₂)ₜR⁹], —(CH₂)ₜ(C═O)O[(CH₂)ₜR⁹], —(CH₂)ₜO(C═O)[(CH₂)ₜR⁹], —(CH₂)ₜ(C₃-C₁₂)carbocyclyl, —(CH₂)ₜ(C₆-C₁₀aryl), and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜ(C═O)N[(CH₂)ₜR⁹]₂, —(CH₂)ₜN[(CH₂)ₜR⁹](C═O)[(CH₂)ₜR⁹], —(CH₂)ₜN[(CH₂)ₜR⁹]S(O)_{w}[(CH₂)ₜR⁹], —(CH₂)ₜR⁹, —(CH₂)ₜO[(CH₂)ₜR⁹], —(CH₂)ₜ(C₃-C₁₂)carbocyclyl, —(CH₂)ₜ(C₆-C₁₀ aryl), and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜ(C═O)N[(CH₂)ₜR⁹]₂, —(CH₂)ₜN[(CH₂)ₜR⁹](C═O)[(CH₂)ₜR⁹], —(CH₂)ₜN[(CH₂)ₜR⁹]S(O)_{w}[(CH₂)ₜR⁹], —(CH₂)ₜR⁹, —(CH₂)ₜO[(CH₂)ₜR⁹], and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜ(C═O )N[(CH₂)ₜR⁹]₂, —(CH₂)ₜN[(CH₂)ₜR⁹](C═O)[(CH₂)ₜR⁹], —(CH₂)ₜR⁹, —(CH₂)ₜO[(CH₂)ₜR⁹], and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜR⁹, —(CH₂)ₜO[(CH₂)ₜR⁹], and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —(CH₂)ₜR⁹, —(CH₂)ₜO[(CH₂)ₜR⁹], and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜOH, —(CH₂)ₜCF₃, —(CH₂)ₜC≡N, —(CH₂)ₜR⁹, and —(CH₂)ₜO[(CH₂)ₜR⁹], wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and -N[(CH₂)ₜR⁹]₂.

In another embodiment the invention relates to a compound of Formula I or pharmaceutically acceptable salt thereof, wherein R¹ is selected from the group consisting of halo, —(CH₂)ₜCF₃, —(CH₂)ₜC≡N, —(CH₂)ₜR⁹, and —(CH₂)ₜO[(CH₂)ₜR⁹], wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜCF₃, —(CH₂)ₜR⁹, and —(CH₂)ₜO[(CH₂)ₜR⁹], wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R¹ is selected from the group consisting of halo, —(CH₂)ₜCF₃, and —(CH₂)ₜR₉, wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In a preferred embodiment the invention relates to a compound of Formula I or pharmaceutically acceptable salt thereof, wherein R¹ is independently selected from the group consisting of halo and —(CH₂)ₜCF₃, wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹₂.

In a more preferred embodiment the invention relates to a compound of Formula I or pharmaceutically acceptable salt thereof, wherein each R¹ is halo.

In a more preferred embodiment the invention relates to a compound of Formula I or pharmaceutically acceptable salt thereof, wherein each R¹ is —(CH₂)ₜCF₃, wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In a more preferred embodiment the invention relates to a compound of Formula I or pharmaceutically acceptable salt thereof, wherein each R¹ is —(CH₂)ₜC≡N, wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another preferred embodiment the invention relates to a compound of Formula I or pharmaceutically acceptable salt thereof, wherein R¹ is —(CH₂)ₜR⁹, wherein each R¹ is —(CH₂)ₜR⁹, wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In a preferred embodiment the invention relates to a compound of Formula I or pharmaceutically acceptable salt thereof, wherein each R¹ is —(CH₂)ₜO[(CH₂)ₜR⁹], wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof wherein A is a 1,3-cyclohexyl and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In yet another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof, wherein R² is selected from the group consisting of hydrogen, —(C₁-C₆)alkyl, —(CH₂)_{q}OH, —(CH₂)_{q}O(C₁-C₆)alkyl, —(CH₂)_{q}O(C₁-C₆)alkylOH, and —(CH₂)ₚCN.

In another embodiment the invention relates to a compound of Formula I or pharmaceutically acceptable salt thereof, wherein R² is selected from the group consisting of hydrogen, —(C₁-C₆)alkyl, —(CH₂)_{q}OH, —(CH₂)_{q}O(C₁-C₆)alkyl, and —(CH₂)ₚCN.

In another embodiment the invention relates to a compound of Formula I or pharmaceutically acceptable salt thereof, wherein R² is selected from the group consisting of hydrogen, —(C₁-C₆)alkyl, —(CH₂)_{q}OH, and —(CH₂)_{q}O(C₁-C₆)alkyl.

In a preferred embodiment the invention relates to a compound of Formula I or pharmaceutically acceptable salt thereof, wherein R² is selected from the group consisting of hydrogen and —(C₁-C₆)alkyl.

In a more preferred embodiment the invention relates to a compound of Formula I or pharmaceutically acceptable salt thereof, wherein R² is hydrogen.

In another preferred embodiment the invention relates to a compound of Formula I or pharmaceutically acceptable salt thereof, wherein R² is —(C₁-C₆)alkyl.

In one embodiment the invention relates to a compound of Formula I or pharmaceutically acceptable salt thereof wherein R¹ is selected from the group consisting of halo and —(CH₂)ₜCF₃ and wherein R² is selected from the group consisting of hydrogen, —(C₁-C₆)alkyl, —(CH₂)_{q}OH, and —(CH₂)_{q}O(C₁-C₆)alkyl wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof wherein A is a 1,3-cyclohexyl, and wherein R² is selected from the group consisting of hydrogen, —(C₁-C₆)alkyl, —(CH₂)_{q}OH, and —(CH₂)_{q}O(C₁-C₆)alkyl wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof wherein A is a bicyclo[3.1.1]heptanyl, and wherein R² is selected from the group consisting of hydrogen, —(C₁-C₆)alkyl, —(CH₂)_{q}OH, and —(CH₂)_{q}O(C₁-C₆)alkyl wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

In another embodiment the invention relates to a compound of Formula I or pharmaceutically acceptable salt thereof wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂ and wherein R² is selected from the group consisting of hydrogen, —(C₁-C₆)alkyl, —(CH₂)_{q}OH, and —(CH₂)_{q}O(C₁-C₆)alkyl.

In another embodiment the invention relates to a compound of Formula I or pharmaceutically acceptable salt thereof of wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, and wherein R² is selected from the group consisting of hydrogen, —(C₁-C₆)alkyl, —(CH₂)_{q}OH, and —(CH₂)_{q}O(C₁-C₆)alkyl.

In another embodiment the invention relates to a compound of Formula I or pharmaceutically acceptable salt thereof, wherein each R³ is independently selected from the group consisting of hydrogen, —CN, halo, hydroxy, —(C₁-C₆)alkyl, —(C₂-C₆)alkenyl, —(C₂-C₆)alkynyl, —(C₁-C₆)alkoxy, —CF₃, —OCF₃, —N[(CH₂)ₜR⁹]₂, —(C═O)O(C₁-C₆)alkyl, —(C═O)(C₁-C₆)alkyl, —(C₃-C₁₂)carbocyclyl, —(CH₂)ₜ(C₆-C₁₀ aryl), —(CH₂)ₜ(4 to 14 membered heterocyclyl), —(CH₂)ₜO(CH₂)ₜ(C₆-C₁₀ aryl), —(CH₂)ₜO(CH₂)ₜ(4 to 14 membered heterocyclyl), —(CH₂)ₜ(C═O)(CH₂)ₜ(C₆-C₁₀ aryl), —(CH₂)ₜ(C═O)(CH₂)ₜ(4 to 14 membered heterocyclyl), wherein said heterocyclyl has 1 to 4 ring heteroatoms selected from the group consisting of N, O, and S, and wherein each said alkyl, cycloalkyl, aryl, or heterocyclyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In another embodiment the invention relates to a compound of Formula I or pharmaceutically acceptable salt thereof, wherein each R³ is independently selected from the group consisting of hydrogen, —CN, halo, hydroxy, —(C₁-C₆)alkyl, —(C₂-C₆)alkenyl, —(C₂-C₆)alkynyl, —(C₁-C₆)alkoxy, —CF₃, —OCF₃, —N[(CH₂)ₜR⁹]₂, —(C═O)O(C₁-C₆)alkyl, —(C═O)(C₁-C₆)alkyl, —(C₃-C₁₂)carbocyclyl, —(CH₂)ₜ(C₆-C₁₀ aryl), —(CH₂)ₜO(CH₂)ₜ(C₆-C₁₀ aryl), and —(CH₂)ₜ(C═O)(CH₂)ₜ(C₆-C₁₀ aryl), wherein said alkyl, alkenyl, carbocyclyl, or aryl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In another embodiment the invention relates to a compound of Formula I or pharmaceutically acceptable salt thereof, wherein each R³ is independently selected from the group consisting of hydrogen, —CN, halo, hydroxy, —(C₁-C₆)alkyl, —(C₂-C₆)alkenyl, —(C₂-C₆)alkynyl, —(C₁-C₆)alkoxy, —CF₃, —OCF₃, —N[(CH₂)ₜR⁹]₂, —(C═O)O(C₁-C₆)alkyl, —(C═O)(C₁-C₆)alkyl, —(C₃-C₁₂)carbocyclyl, —(CH₂)ₜ(C₆-C₁₀ aryl), —(CH₂)ₜO(CH₂)ₜ(C₆-C₁₀ aryl), and —(CH₂)ₜ(C═O)(CH₂)ₜ(C₆-C₁₀ aryl), wherein said alkyl, alkenyl, carbocyclyl, or aryl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl,

In another embodiment the invention relates to a compound of Formula I or pharmaceutically acceptable salt thereof, wherein each R³ is independently selected from the group consisting of hydrogen, —CN, halo, hydroxy, —(C₁-C₆)alkyl, —(C₂-C₆)alkenyl, —(C₂-C₆)alkynyl, —(C₁-C₆)alkoxy, —CF₃, —OCF₃, —N[(CH₂)ₜR⁹]₂, —(C═O)O(C₁-C₆)alkyl, and —(C═O)(C₁-C₆)alkyl, wherein said alkyl or alkenyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)OC₁-C_{C}₆)alkyl, —(C═O)(C₁-C₆)alkyl, and —(C₃-C₁₂)carbocyclyl.

In another embodiment the invention relates to a compound of Formula I or pharmaceutically acceptable salt thereof, wherein each R³ is independently selected from the group consisting of hydrogen, —CN, halo, hydroxy, —(C₁-C₆)alkyl, —(C₁-C₆)alkoxy, —CF₃, —OCF₃, —N[((CH₂)ₜR⁹]₂, —(C═O)O(C₁-C₆)alkyl, and —(C═O)(C₁-C₆)alkyl, wherein said alkyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In another embodiment the invention relates to a compound of Formula I or pharmaceutically acceptable salt thereof, wherein each R³ is independently selected from the group consisting of hydrogen, halo, hydroxy, —(C₁-C₆)alkyl, —(C₁-C₆)alkoxy, —CF₃, —OCF₃, —(C═O)O(C₁-C₆)alkyl, and —(C═O)(C₁-C₆)alkyl, wherein said alkyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In another embodiment the invention relates to a compound of Formula I or pharmaceutically acceptable salt thereof, wherein each R³ is independently selected from the group consisting of hydrogen, halo, hydroxy, —(C₁-C₆)alkyl, —(C₁-C₆)alkoxy, —CF₃, and —OCF₃, wherein said alkyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In another embodiment the invention relates to a compound of Formula I or pharmaceutically acceptable salt thereof, wherein each R³ is independently selected from the group consisting of hydrogen, halo, hydroxy, —(C₁-C₆)alkyl, and —(C₁-C₆)alkoxy, wherein said alkyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In another embodiment the invention relates to a compound of Formula I or pharmaceutically acceptable salt thereof, wherein each R³ is independently selected from the group consisting of hydrogen, halo, and —(C₁-C₆)alkyl, wherein said alkyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In a preferred embodiment the invention relates to a compound of Formula I or pharmaceutically acceptable salt thereof, each R³ is independently —(C₁-C₆)alkyl, wherein said alkyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl.

In a preferred embodiment the invention relates to a compound of Formula I or pharmaceutically acceptable salt thereof, wherein each R³ is halo.

In a preferred embodiment the invention relates to a compound of Formula I or pharmaceutically acceptable salt thereof, wherein each R³ is hydrogen.

In another embodiment the invention relates to a compound of Formula I or pharmaceutically acceptable salt thereof, wherein R⁴ is selected from the group consisting of hydrogen, —(C₁-C₆)alkyl, —(CH₂)_{q}OH, —(CH₂)_{q}O(C₁-C₆)alkyl, —(CH₂)_{q}O(C₁-C₆)alkylOH, —(CH₂)ₚCF₃, and —(CH₂)ₚCN.

In another embodiment the invention relates to a compound of Formula I or pharmaceutically acceptable salt thereof, wherein R⁴ is selected from the group consisting of hydrogen, —(C₁-C₆)alkyl, —(CH₂)_{q}OH, —(CH₂)_{q}O(C₁-C₆)alkyl, —(CH₂)_{q}O(C₁-C₆)alkylOH, and —(CH₂)ₚCF₃.

In another embodiment the invention relates to a compound of Formula I or pharmaceutically acceptable salt thereof, wherein R⁴ is selected from the group consisting of hydrogen and —(C₁-C₆)alkyl.

In a preferred embodiment the invention relates to a compound of Formula I or pharmaceutically acceptable salt thereof, wherein R⁴ is hydrogen.

In another embodiment the invention relates to a compound of Formula I or pharmaceutically acceptable salt thereof, wherein R⁴ is —(C₁-C₆)alkyl.

In a preferred embodiment the invention relates to a compound of Formula I or pharmaceutically acceptable salt thereof, wherein R⁴ is propyl.

In a preferred embodiment the invention relates to a compound of Formula I or pharmaceutically acceptable salt thereof, wherein R⁴ is ethyl.

In a preferred embodiment the invention relates to a compound of Formula I or pharmaceutically acceptable salt thereof, wherein R⁴ is methyl.

In another embodiment the invention relates to a compound of Formula I or pharmaceutically acceptable salt thereof wherein A is a 1,3-cyclohexyl, and wherein R⁴ is selected from the group consisting of hydrogen and —(C₁-C₆)alkyl.

In another embodiment the invention relates to a compound of Formula I or pharmaceutically acceptable salt thereof wherein A is a bicyclo[3.1.1]heptanyl, and wherein R⁴ is selected from the group consisting of hydrogen and —(C₁-C₆)alkyl.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, and wherein R⁴ is selected from the group consisting of hydrogen and —(C₁-C₆)alkyl.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, and wherein R⁴ is selected from the group consisting of hydrogen and —(C₁-C₆)alkyl.

In another embodiment the invention relates to a compound of Formula I or pharmaceutically acceptable salt thereof, wherein R⁵ is selected from the group consisting of —(CH₂)ₜ(C₃-C₁₂)carbocyclyl, —(CH₂)ₜ(C₆-C₁₀)aryl, —(CH₂)ₚ(C₁-C₆)alkoxy, —(CH₂)ₜO(CH₂)ₜ(C₆-C₁₀)aryl, —(CH₂)ₜN[(CH₂)ₜR⁹]₂, —(CH₂)ₜN[(CH₂)ₜR⁹](C₆-C₁₀)aryl, —(CH₂)ₜ(4 to 14 membered heterocyclyl), —(CH₂)ₜO(CH₂)ₜ(4 to 14 membered heterocyclyl) and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said carbocyclyl, aryl, and heterocyclyl are independently optionally substituted by 1 to 3 substituents selected from R⁶.

In another embodiment the invention relates to a compound of Formula I or pharmaceutically acceptable salt thereof, wherein R⁵ is selected from the group consisting of —(CH₂)ₜ(C₃-C₁₂)carbocyclyl, —(CH₂)ₜ(C₆-C₁₀)aryl, —(CH₂)ₜO(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), —(CH₂)ₜO(CH₂)ₜ(4 to 14 membered heterocyclyl) and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said carbocyclyl, aryl, and heterocyclyl are independently optionally substituted by 1 to 3 substituents selected from R⁶.

In another embodiment the invention relates to a compound of Formula I or pharmaceutically acceptable salt thereof, wherein R⁵ is selected from the group consisting of —(C H₂)ₜ(C₆-C₁₀)aryl, —(CH₂)ₜO(CH₂)ₜ(C₆-C₁₀)aryl, —(CH₂)ₜN[(CH₂)ₜR⁹](C₆-C₁₀)aryl, —(CH₂)ₜ(4to 14 membered heterocyclyl), —(CH₂)ₜO(CH₂)ₜ(4 to 14 membered heterocyclyl) and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In a preferred embodiment the invention relates to a compound of Formula I or pharmaceutically acceptable salt thereof, wherein R⁵ is —(CH₂)ₜ(C₆-C₁₀)aryl, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In a preferred embodiment the invention relates to a compound of Formula I or pharmaceutically acceptable salt thereof wherein R⁵ is —(CH₂)ₜ(C₆-C₁₀)aryl, wherein each said (CH₂) moiety may optionally be substituted by one to two substituents independently selected substituents selected from R⁶, and wherein each said aryl and heterocyclyl are independently optionally substituted by 1 to 3 substituents selected from R⁶ and is selected from the group consisting of:

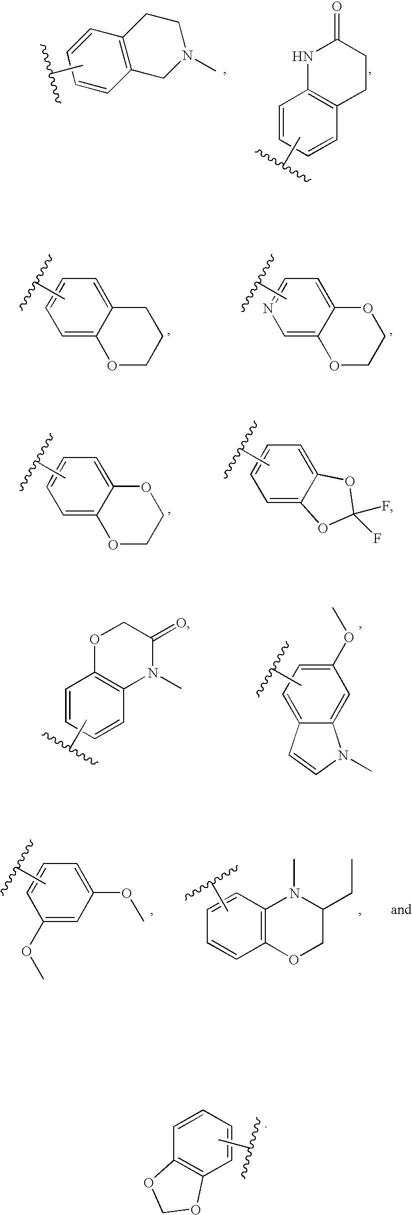

In a preferred embodiment the invention relates to a compound of Formula I or pharmaceutically acceptable salt thereof wherein R⁵ is —(CH₂)ₜ(C₆-C₁₀)aryl, wherein each said (CH₂) moiety may optionally be substituted by one to two substituents independently selected substituents selected from R⁶, and wherein each said aryl and heterocyclyl are independently optionally substituted by 1 to 3 substituents selected from R⁶ and is selected from the group consisting of:

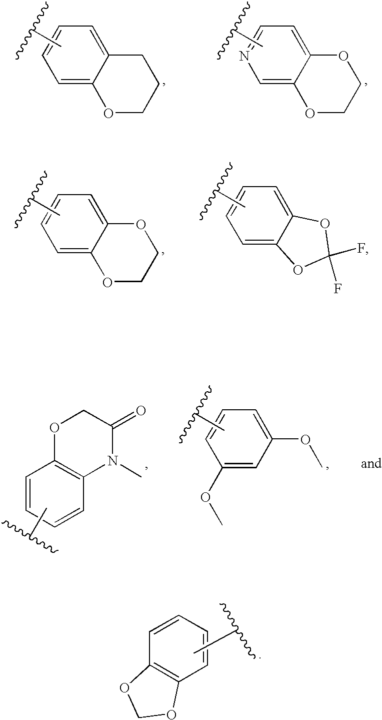

In a preferred embodiment the invention relates to a compound of Formula I or pharmaceutically acceptable salt thereof wherein R⁵ is —(CH₂)ₜ(C₆-C₁₀)aryl, wherein each said (CH₂) moiety may optionally be substituted by one to two substituents independently selected substituents selected from R⁶, and wherein each said aryl and heterocyclyl are independently optionally substituted by 1 to 3 substituents selected from R⁶ and is selected from the group consisting of:

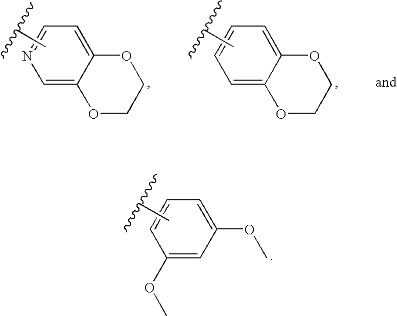

In a preferred embodiment the invention relates to a compound of Formula I or pharmaceutically acceptable salt thereof, wherein R⁵ is —(CH₂)ₜN[(CH₂)ₜR⁹](C₆-C₁₀)aryl, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In a preferred embodiment the invention relates to a compound of Formula I or pharmaceutically acceptable salt thereof, wherein R⁵ is —(CH₂)ₜ(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, and wherein each said (CH₂) moiety may optionally be substituted by one to two substituents independently selected substituents selected from R⁶.

In a preferred embodiment the invention relates to a compound of Formula I or pharmaceutically acceptable salt thereof, wherein R⁵ is —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein each said (CH₂) moiety may optionally be substituted by one to two substituents independently selected substituents selected from R⁶.

In another embodiment the invention relates to a compound of Formula I or pharmaceutically acceptable salt thereof of any of the preceding claims wherein R⁴ is selected from the group consisting of hydrogen and —(C₁-C₆)alkyl and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof of any of the preceding claims wherein A is a 1,3-cyclohexyl, and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof of any of the preceding claims wherein A is a bicyclo[3.1.1]heptanyl, and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof wherein A is a 1,3-cyclohexyl, and wherein R⁴ is selected from the group consisting of hydrogen and —(C₁-C₆)alkyl, and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof wherein A is a bicyclo[3.1.1]heptanyl, and wherein R⁴ is selected from the group consisting of hydrogen and —(C₁-C₆)alkyl, and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹](4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

In another embodiment the invention relates to a compound of Formula I or pharmaceutically acceptable salt thereof, wherein each R⁶ is independently selected from the group consisting of halo, —NO₂, —OR⁷, —(CH₂)ₜ(R⁷), —CF₃, —OCF₃, —OCHF₂, —OCH₂F, —O(CH₂)ₜ(C₆-C₁₀)aryl(R⁷), —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜSR⁷, —(CH₂)ₜ(S═O)R⁷, —(CH₂)ₜS(═O)₂R⁷, —[C(R⁶)₂]ₜN(R⁷)S(═O)₂R⁷, —S(═O)₂N(R⁷)₂, —N(R⁷)₂, —(C═O)R⁷, —(C═O)OR⁷, —[C(R⁷)₂]ₜO(C═O)R⁷, —[C(R⁷)₂]ₜO(C═O)N(R⁷)₂, —[C(R⁷)₂]ₜN(R⁷)(C═O)R⁷, —[C(R⁷)₂]ₜN(R⁷)₂, —[C(R⁷)₂]ₜOR⁷, —[C(R⁷)₂]ₜN(R⁷)(C═O)OR⁷, —[C(R⁷)₂]ₜN(R⁷)(C═O)N(R⁷)₂, —[C(R⁷)₂]ₜN(R⁷)S(═O)₂N(R⁷)₂, —[C(R⁷)₂]ₜN(R⁷)₂, and —(C═O)N(R⁷)₂.

In another embodiment the invention relates to a compound of Formula I or pharmaceutically acceptable salt thereof, wherein each R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof, wherein each R⁶ is independently selected from the group consisting of —OR⁷, —(C ₂)ₜ(R⁷), —(CH₂)ₜC≡N, —(C₁-C₆)alkyl, —(CH₂)ₜ(C₃-C₁₂)carbocyclyl(R⁷), and —(C═O)R⁷.

In another embodiment the invention relates to a compound of Formula I or pharmaceutically acceptable salt thereof wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl ring has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof wherein A is a 1,3-cyclohexyl, and wherein R⁴ is selected from the group consisting of hydrogen and —(C₁-C₆)alkyl, and wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), ——(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O )R⁷, —(C(R⁷)₂]ₜOR⁷, and —(C═O)N(R⁷)₂.

In another embodiment the invention relates to a compound of Formula I or pharmaceutically acceptable salt thereof wherein A is a bicyclo[3.1.1]heptanyl, and wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂ wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, and wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C6)alkyl, wherein R⁴ is selected from the group consisting of hydrogen and —(C₁-C6)alkyl, and wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(C₂)ₜ(C₆-C₁₀)aryl(R⁷), —(C₂)ₜ(4 to 14 membered heterocyclyl)(R⁷), —(CH₂)ₜ(S═O)R⁷, —N(R⁷)₂, —(C═O )R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof of wherein A is a 1,3-cyclohexyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷, —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R₇, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof of wherein A is a bicyclo[3.1.1]heptanyl, said ring may optionally contain 1 or 2 double bonds, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein R⁶ is independently selected from the group consisting of halo, —OR⁷, —(CH₂)_{t(R}⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR₉]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein R⁶ is independently selected from the group consisting of halo, —OR⁷, —(CH₂)ₜ(R⁷), —CF₃, —(CH₂)ₜC≡N, —(C₁-C₆)alkyl, —(C₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof of wherein A is a bicyclo[3.1.1]heptanyl, said ring may optionally contain 1 or 2 double bonds, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂.

In another embodiment the invention relates to a compound of Formula I or pharmaceutically acceptable salt thereof, wherein each R⁷ is independently selected from the group consisting of H, —CF₃, —(C₁-C₆)alkyl, —(C₆-C₁₀)aryl, or two R⁷ groups on the same nitrogen atom may be taken together with the nitrogen atom to form a 5 to 8 membered heterocyclyl ring, wherein said heterocyclyl ring has 1 to 3 ring heteroatoms selected from the group consisting of N, O, and S, or two R⁷ groups on the same carbon atom may be taken together with the carbon atom to form a 3 to 7 membered carbocyclyl ring and wherein said alkyl, cycloalkyl, aryl, heterocyclyl and carbocyclyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —C(═O)O(C₁-C₆)alkyl, —C(═O)(C₁-C₆)alkyl, and —(C₃-C₁₂)carbocyclyl.

In another embodiment the invention relates to a compound of Formula I or pharmaceutically acceptable salt thereof, wherein each R⁷ is independently selected from the group consisting of H, —CF₃, —(C₁-C₆)alkyl, —(C₆-C₁₀)aryl, wherein said alkyl, cycloalkyl, aryl and carbocyclyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —C(═O)(C₁-C₆)alkyl, and —(C3-C₁₂)carbocyclyl.

In a preferred embodiment the invention relates to a compound of Formula I or pharmaceutically acceptable salt thereof, wherein each R⁷ is H.

In a preferred embodiment the invention relates to a compound of Formula I or pharmaceutically acceptable salt thereof, wherein each R⁷ is —CF₃.

In a preferred embodiment the invention relates to a compound of Formula I or pharmaceutically acceptable salt thereof, wherein each R⁷ is —(C₁-C₆)alkyl, wherein said alkyl, may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —C(═O)(C₁-C₆)alkyl, and —(C₃-C₁₂)carbocyclyl.

In a preferred embodiment the invention relates to a compound of Formula I or pharmaceutically acceptable salt thereof, wherein each R⁷ is —(C₆-C₁₀)aryl, wherein said aryl, may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —C(═O)(C₁-C₆)alkyl, and —(C₃-C₁₂)carbocyclyl.

In another embodiment the invention relates to a compound of Formula I or pharmaceutically acceptable salt thereof, wherein X is selected from the group consisting of O and NR⁸.

In a preferred embodiment the invention relates to a compound of Formula I or pharmaceutically acceptable salt thereof, wherein X is O.

In another preferred embodiment the invention relates to a compound of Formula I or pharmaceutically acceptable salt thereof, wherein X is NR⁸.

In another embodiment the invention relates to a compound of Formula I or pharmaceutically acceptable salt thereof wherein A is a 1,3-cyclohexyl, wherein X is O.

In another embodiment the invention relates to a compound of Formula I or pharmaceutically acceptable salt thereof wherein A is a bicyclo[3.1.1]heptanyl, and wherein X is O.

In another embodiment the invention relates to a compound of Formula I or pharmaceutically acceptable salt thereof wherein A is a 1,3-cyclohexyl, wherein X is NR⁸.

In another embodiment the invention relates to a compound of Formula I or pharmaceutically acceptable salt thereof wherein A is a bicyclo[3.1.1]heptanyl, and wherein X is NR⁸.

In another embodiment the invention relates to a compound of Formula I or pharmaceutically acceptable salt thereof wherein A is a 1,3-cyclohexyl, wherein R⁴ is selected from the group consisting of hydrogen and —(C₁-C₆)alkyl, and wherein X is O.

In another embodiment the invention relates to a compound of Formula I or pharmaceutically acceptable salt thereof wherein A is a bicyclo[3.1.1]heptanyl, wherein R⁴ is selected from the group consisting of hydrogen and —(C₁-C₆)alkyl, and wherein X is O.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂ wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, and wherein X is O.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, and wherein X is O.

In another embodiment the invention relates to a compound of Formula I or pharmaceutically acceptable salt thereof of any of the preceding claims wherein A is a 1,3-cyclohexyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is O.

In another embodiment the invention relates to a compound of Formula I or pharmaceutically acceptable salt thereof of any of the preceding claims wherein A is a bicyclo[3.1.1]heptanyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is O.

In another embodiment the invention relates to a compound of Formula I or pharmaceutically acceptable salt thereof wherein A is a 1,3-cyclohexyl, and wherein R⁴ is selected from the group consisting of hydrogen and —(C₁-C₆)alkyl, wherein R⁵ is selected from the group consisting of —(CH₂)ₜ(C₅-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is O.

In another embodiment the invention relates to a compound of Formula I or pharmaceutically acceptable salt thereof wherein A is a bicyclo[3.1.1]heptanyl, and wherein R⁴ is selected from the group consisting of hydrogen and —(C₁-C₆)alkyl, wherein R₅ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is O.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is O.

In another embodiment the invention relates to a compound of Formula I or pharmaceutically acceptable salt thereof wherein A is a 1,3-cyclohexyl, wherein R⁴ is selected from the group consisting of hydrogen and —(C₁-C₆)alkyl, wherein X is NR⁸.

In another embodiment the invention relates to a compound of Formula I or pharmaceutically acceptable salt thereof wherein A is a bicyclo[3.1.1]heptanyl, wherein R⁴ is selected from the group consisting of hydrogen and —(C₁-C₆)alkyl, and wherein X is NR⁸.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, and wherein X is NR⁸.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, and wherein X is NR⁸.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is NR⁸.

In another embodiment the invention relates to a compound of Formula I or pharmaceutically acceptable salt thereof of any of the preceding claims wherein A is a 1,3-cyclohexyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is NR⁸.

In another embodiment the invention relates to a compound of Formula I or pharmaceutically acceptable salt thereof of any of the preceding claims wherein A is a bicyclo[3.1.1]heptanyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and and wherein X is NR⁸.

In another embodiment the invention relates to a compound of Formula I or pharmaceutically acceptable salt thereof wherein A is a 1,3-cyclohexyl, and wherein R⁴ is selected from the group consisting of hydrogen and —(C₁-C₆)alkyl, and wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is NR⁸.

In another embodiment the invention relates to a compound of Formula I or pharmaceutically acceptable salt thereof wherein A is a bicyclo[3.1.1]heptanyl, and wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is NR⁶.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is NR⁶.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, and wherein X is NR⁸.

XXXXX In another embodiment the invention relates to a compound of Formula I or pharmaceutically acceptable salt thereof wherein A is a 1,3-cyclohexyl, and wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is

In another embodiment the invention relates to a compound of Formula I or pharmaceutically acceptable salt thereof wherein A is a bicyclo[3.1.1]heptanyl, and wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is O.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is O.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆ )alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is O.

In another embodiment the invention relates to a compound of Formula I or pharmaceutically acceptable salt thereof of wherein A is a 1,3-cyclohexyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is O.

In another embodiment the invention relates to a compound of Formula I or pharmaceutically acceptable salt thereof of wherein A is a bicyclo[3.1.1]heptanyl, said ring may optionally contain 1 or 2 double bonds, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is O.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is O.

In another embodiment the invention relates to a compound of Formula I or pharmaceutically acceptable salt thereof of wherein A is a bicyclo[3.1.1]heptanyl, said ring may optionally contain 1 or 2 double bonds, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is O.

In another embodiment the invention relates to a compound of Formula I or pharmaceutically acceptable salt thereof wherein A is a 1,3-cyclohexyl, and wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is NR⁸.

In another embodiment the invention relates to a compound of Formula I or pharmaceutically acceptable salt thereof wherein A is a bicyclo[3.1.1]heptanyl, and wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is NR⁸.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R₇, —(C═O)OR⁷, —[C(R⁷)₂]ₜN(R⁷)(C═O)R⁷, —[C(R⁷)₂]ₜOR⁷, and —(C═O)N(R⁷)₂, wherein X is NR⁸.

In another embodiment the invention relates to a compound of Formula I or pharmaceutically acceptable salt thereof wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein Re is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is NR⁸.

In another embodiment the invention relates to a compound of Formula I or pharmaceutically acceptable salt thereof of wherein A is a 1,3-cyclohexyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is NR⁸.

In another embodiment the invention relates to a compound of Formula I or pharmaceutically acceptable salt thereof of wherein A is a bicyclo[3.1.1]heptanyl, said ring may optionally contain 1 or 2 double bonds, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC═N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is NR⁸.

In another embodiment the invention relates to a compound of Formula I or pharmaceutically acceptable salt thereof of wherein A is a 1,3-cyclohexyl, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₈-C₁₀)aryl, —(CH₂)ₜ(4 to 14 membered heterocyclyl), and —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R⁷, —[C(R⁷)₂]ₜOR⁷, and —(C═O)N(R⁷)₂, and wherein X is NR⁸.

In another embodiment the invention relates to a compound of Formula I or pharmaceutically acceptable salt thereof of wherein A is a bicyclo[3.1.1]heptanyl, said ring may optionally contain 1 or 2 double bonds, wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂, wherein R² is selected from the group consisting of hydrogen, —(C₁-C₆)alkyl, —(CH₂)_{q}OH, and —(CH₂)_{q}O(C₁-C₆)alkyl, wherein R⁴ is selected from the group consisting of hydrogen and —(C₁-C₆)alkyl, wherein R⁵ is selected from the group consisting of —(CH₂)ₜ(C₆-C₁₀)aryl, —(CH₂)ₜN[(CH₂)ₜR⁹](C₆-C₁₀)aryl, —(CH₂)ₜ(4 to 14 membered heterocyclyl), a —(CH₂)ₜ(N[(CH₂)ₜR⁹])(4 to 14 membered heterocyclyl), wherein said heterocyclyl has 1 to 3 ring heteroatoms selected from the group consisiting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said aryl and heterocyclyl are independently optionally substituted by 1 to 3 substituents selected from R⁶, wherein R⁶ is independently selected from the group consisting of halo, —OR⁷, —(CH₂)ₜ(R⁷), —CF₃, —(CH₂)ₜC≡N, —(C₁-C₆)alkyl, —(CH₂)ₜ(C₃-C₁₂)carbocyclyl(R⁷), —(CH₂)ₜ, (C₆-C₁₀)aryl(R⁷), —(CH₂)ₜ(4 to 14 membered heterocyclyl)(R⁷), —(CH₂)ₜ(S═O)R⁷, —N(R⁷)₂, —(C═O)R⁷, —(C═O)OR⁷, —[C(R⁷)₂]ₜN(R⁷)(C═O)R₇, —[C(R⁷)₂]ₜOR⁷, and —(C═O)N(R⁷)₂, and wherein X is NR⁸.

In another embodiment the invention relates to a compound of Formula I or pharmaceutically acceptable salt thereof, wherein R⁶ is selected from the group consisting of hydrogen and —(C₁-C₆)alkyl.

In a preferred embodiment the invention relates to a compound of Formula I or pharmaceutically acceptable salt thereof, wherein R⁸ is hydrogen.

In a preferred embodiment the invention relates to a compound of Formula I or pharmaceutically acceptable salt thereof, wherein R⁸ is —(C₁-C₆)alkyl.

In another embodiment the invention relates to a compound of Formula I or pharmaceutically acceptable salt thereof, wherein each R⁹ is independently selected from the group consisting of hydrogen, —(C₁-C₆)alkyl, —(CH₂)ₜ(C₆-C₁₀ aryl), —(CH₂)ₜ(C₃-C₁₂)carbocyclyl, and —(CH₂)ₜ(4 to 14 membered heterocyclyl), wherein said heterocyclyl has 1 to 3 ring heteroatoms selected from the group consisting of N, O, and S, or two R⁹ groups on the same nitrogen atom may be taken together with the nitrogen atom to form a 5 to 8 membered heterocyclyl ring wherein said heterocyclyl ring optionally has 1 to 3 ring additional heteroatoms selected from the group consisting of N, O, and S, or two R⁹ groups on the same carbon atom may be taken together with the carbon atom to form a 3 to 7 membered carbocyclyl ring, wherein each said alkyl, aryl, (CH₂) moiety, carbocyclyl, and heterocyclyl may optionally be substituted by one to three substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl ring has 1 to 3 ring heteroatoms selected from the group consisting of N, O, and S.

In another embodiment the invention relates to a compound of Formula I or pharmaceutically acceptable salt thereof, wherein each R⁹ is —(CH₂)ₜ(4 to 14 membered heterocyclyl), wherein said heterocyclyl has 1 to 3 ring heteroatoms selected from the group consisting of N, O, and S, or two R⁹ groups on the same nitrogen atom may be taken together with the nitrogen atom to form a 5 to 8 membered heterocyclyl ring wherein said heterocyclyl ring optionally has 1 to 3 ring additional heteroatoms selected from the group consisiting of N, O, and S, or two R⁹ groups on the same carbon atom may be taken together with the carbon atom to form a 3 to 7 membered carbocyclyl ring, wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S, and wherein each said heterocyclyl may optionally be substituted by one to three substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S.

In another embodiment the invention relates to a compound of Formula I or pharmaceutically acceptable salt thereof, wherein each R⁹ is —(CH₂)ₜ(C₃-C₁₂)carbocyclyl, wherein two R⁹ groups on the same carbon atom may be taken together with the carbon atom to form a 3 to 7 membered carbocyclyl ring, wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S, and wherein each said carbocyclyl may optionally be substituted by one to three substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S.

In another embodiment the invention relates to a compound of Formula I or pharmaceutically acceptable salt thereof, wherein each R⁹ is —(CH₂)ₜ(C₆-C₁₀ aryl), wherein two R⁹ groups on the same carbon atom may be taken together with the carbon atom to form a 3 to 7 membered carbocyclyl ring, wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S, and wherein each said aryl may optionally be substituted by one to three substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 and 14 membered heterocyclyl) wherein said heterocyclyl has 1 to 3 ring heteroatoms selected from the group consisting of N, O and S.

In a preferred embodiment the invention relates to a compound of Formula I or pharmaceutically acceptable salt thereof, wherein each R⁹ is —(C₁-C₆)alkyl, wherein two R⁹ groups on the same carbon atom may be taken together with the carbon atom to form a 3 to 7 membered carbocyclyl ring, wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S, and wherein each said alkyl may optionally be substituted by one to three substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S.

In a preferred embodiment the invention relates to a compound of Formula I or pharmaceutically acceptable salt thereof, wherein each R⁹ is hydrogen.

In another embodiment the invention relates to a compound of Formula I or pharmaceutically acceptable salt thereof, wherein each p is an integer independently selected from 1, 2, or 3.

In another embodiment the invention relates to a compound of Formula I or pharmaceutically acceptable salt thereof, wherein each p is an integer independently selected from 1 or 2.

In another embodiment the invention relates to a compound of Formula I or pharmaceutically acceptable salt thereof, wherein p is 1.

In another embodiment the invention relates to a compound of Formula I or pharmaceutically acceptable salt thereof, wherein p is 2.

In another embodiment the invention relates to a compound of Formula I or pharmaceutically acceptable salt thereof, wherein each t is an integer independently selected from 0, 1, 2, 3, 4, or 5.

In another embodiment the invention relates to a compound of Formula I or pharmaceutically acceptable salt thereof, wherein each t is an integer independently selected from 0,1, 2, or 3.

In another embodiment the invention relates to a compound of Formula I or pharmaceutically acceptable salt thereof, wherein each t is an integer independently selected from 0, 1 or 2.

In another embodiment the invention relates to a compound of Formula I or pharmaceutically acceptable salt thereof, wherein t is 2.

In another embodiment the invention relates to a compound of Formula I or pharmaceutically acceptable salt thereof, wherein t is 1.

In another embodiment the invention relates to a compound of Formula I or pharmaceutically acceptable salt thereof, wherein t is 0.

In another embodiment the invention relates to a compound of Formula I or pharmaceutically acceptable salt thereof, wherein each n is an integer independently selected from 0, 1, 2, or 3.

In another embodiment the invention relates to a compound of Formula I or pharmaceutically acceptable salt thereof, wherein each n is an integer independently selected from 0, 1, or 2.

In another embodiment the invention relates to a compound of Formula I or pharmaceutically acceptable salt thereof, wherein n is 2.

In another embodiment the invention relates to a compound of Formula I or pharmaceutically acceptable salt thereof, wherein n is 1.

In another embodiment the invention relates to a compound of Formula I or pharmaceutically acceptable salt thereof, wherein n is 0.

In another embodiment the invention relates to a compound of Formula I or pharmaceutically acceptable salt thereof, wherein each q is an integer independently selected from 2, 3, or 4.

In another embodiment the invention relates to a compound of Formula I or pharmaceutically acceptable salt thereof, wherein q is 4.

In another embodiment the invention relates to a compound of Formula I or pharmaceutically acceptable salt thereof, wherein q is 3.

In another embodiment the invention relates to a compound of Formula I or pharmaceutically acceptable salt thereof, wherein q is 2.

In another embodiment the invention relates to a compound of Formula I or pharmaceutically acceptable salt thereof, wherein each w is an integer independently selected from 0 or 1.

In another embodiment the invention relates to a compound of Formula I or pharmaceutically acceptable salt thereof, wherein w is 1.

In another embodiment the invention relates to a compound of Formula I or pharmaceutically acceptable salt thereof, wherein w is 0.

In another embodiment the invention relates to a compound of Formula I or pharmaceutically acceptable salt thereof, wherein each z is an integer independently selected from 0, 1, 2, 3, 4, or 5.

In another embodiment the invention relates to a compound of Formula I or pharmaceutically acceptable salt thereof, wherein each z is an integer independently selected from 0, 1, 2, or 3.

In another embodiment the invention relates to a compound of Formula I or pharmaceutically acceptable salt thereof, wherein z is 2.

In another embodiment the invention relates to a compound of Formula I or pharmaceutically acceptable salt thereof, wherein z is 1.

In another embodiment the invention relates to a compound of Formula I or pharmaceutically acceptable salt thereof, wherein z is 0.

It will be understood by those of skill in the art that the above identified embodiments may be taken together in a variety of combinations.

In an embodiment of the present invention, the compound is selected from the group consisting of:

N-((1R,3S)-3-(1H-benzo[d]imidazol-2-yl)cyclohexyl)-2,3-dihydro-[1,4]dioxino[2,3-dihydro-[1,4]dioxino[2,3-c]pyridine-7-carboxamide,

1-[(1R,3S)-3-(1H-benzimidazol-2-yl)cyclohexyl]-3-[4-chloro-3-(trifluoromethyl)phenyl]urea,

N-{3-[6-(trifluoromethyl)-1H-benzimidazol-2-yl]cyclohexyl}-2,3-dihydro-1,4-benzodioxine-6-carboxamide,

N-{3-[6-(dimethylamino)-1H-benzimidazol-2-yl]cyclohexyl}-2,3-dihydro-1,4-benzodioxine-6-carboxamide,

N-((3S)-3-(1H-benzo[d]imidazol-2-yl)cyclohexyl)-2,3-dihydrobenzo[b][1,4]dioxine-6-carbothioamide,

N-[(1R,3S)-3-(5-chloro-1H-benzimidazol-2-yl)cyclohexyl]-1-methyl-2-oxo-1,2,3,4-tetrahydroquinoline-6-carboxamide,

4-methyl-3-oxo-N-{(1R,3S)-3-[5-(trifluoromethyl)-1H-benzimidazol-2-yl]3cyclohexyl}-3,4-dihydro-2H-1,4-benzoxazine-6-carboxamide,

1-[(1R,3S )-3-(1H-benzimidazol-2-yl)cyclohexyl]-3-(3,5-dichlorophenyl)urea, N-[(1R,3S)-3-(5-chloro-1H-benzimidazol-2-yl)cyclohexyl]-3,5-dimethoxybenzamide,

N-[(1R,3S)-3-(5-chloro-1H-benzimidazol-2-yl)cyclohexyl]-2-oxo-1,2,3,4-tetrahydroquinoline-6-carboxamide,

1-[(1R,3S)-3-(1H-benzimidazol-2-yl)cyclohexyl]-3-(3,4-dichlorophenyl)urea,

N-[3-(6-{[(2-methoxyethyl)amino]methyl}-1-methyl-1H-benzimidazol-2-yl)cyclohexyl]-2,3-dihydro-1,4-benzodioxine-6-carboxamide,

1,3-dimethyl-N-{(1R,3S)-3-[5-(trifluoromethyl)-1H-benzimidazol-2-yl]cyclohexyl}-1H-thieno[2,3-c]pyrazole-5-carboxamide,

2-oxo-N-{(1R,3S)-3-[5-(trifluoromethyl)-1H-benzimidazol-2-yl]cyclohexyl}-1,2,3,4-tetrahydroquinoline-6-carboxamide,

4-benzoyl-N-{5-[5-(trifluoromethyl)-1H-benzimidazol-2-yl]bicyclo[3.1.1]hept-1-yl}benzamide,

3,5-dimethoxy-N-{(1R,3S)-3-[6-(trifluoromethyl)-1H-benzimidazol-2-yl]cyclohexyl}benzamide,

1-methyl-2-oxo-N-{(1R,3S)-3-[5-(trifluoromethyl)-1H-benzimidazol-2-yl]cyclohexyl}-1,2,3,4-tetrahydroquinoline-6-carboxamide,

N-[3-(6-bromo-1H-benzimidazol-2-yl)cyclohexyl]-2,3-dihydro-1,4-benzodioxine-6-carboxamide,

3,5-dimethoxy-N-{5-[5-(trifluoromethyl)-1H-benzimidazol-2-yl]bicyclo[3.1.1]hept-1-yl}benzamide,

1-[(1R,3S)-3-(1H-benzimidazol-2-yl)cyclohexyll-3-(4-isopropylphenyl)urea,

N-[(1R,3S)-3-(1-methyl-1H-benzimidazol-2-yl)cyclohexyl]-2,3-dihydro-1,4-benzodioxine-6-carboxamide,

N-{5-[5-(trifluoromethyl)-1H-benzimidazol-2-yl]bicyclo[3.1.1]hept-1-yl}-2,3-dihydro-1,4-benzodioxine-6-carboxamide,

N-((1S, 3S)-3-(1H-benzo[d]imidazol-2-yl)cyclohexyl)-3-oxo-3,4-dihydro-2H-benzo[b][1,4]oxazine-6-carboxamide,

N-[3-(6-bromo-1-methyl-1H-benzimidazol-2-yl)cyclohexyl]-3,5-dimethoxybenzamide,

4-(trifluoroacetyl)-N-{5-[5-(trifluoromethyl)-1H-benzimidazol-2-yl]bicyclo[3.1.1]hept-1-yl}benzamide,

N-[(1R,3S)-3-(5-chloro-1H-benzimidazol-2-yl)cyclohexyl]-4-methyl-3-oxo-3,4-dihydro-2H-1,4-benzoxazine-6-carboxamide,

1-(4-cyanophenyl)-3-{5-[5-(trifluoromethyl)-1H-benzimidazol-2-yl]bicyclo[3.1.1]hept-1-yl}urea,

3,5-dimethoxy-N-{3-[6-(trifluoromethyl)-1H-benzimidazol-2-yl]cyclahexyl}benzamide,

N-[3-(1H-benzimidazol-2-yl)cyclohexyl]-2-(5-chloro-1H-benzimidazol-2-yl)acetamide,

N-{(3S )-3-[5-(trifluoromethyl )-1H-benzimidazol-2-yl]cyclohexyl}-2,3-dihydro-1,4-benzodioxine-6-carboxamide,

N-[(1R,3S)-3-(6-chloro-1H-benzimidazol-2-yl)cyclohexyl]-2,3-dihydro-1,4-benzodioxine-6-carboxamide,

1-[(1R, 3S)-3-(1H-benzimidazol-2-yl)cyclohexyl]-3-[4-(trifluoromethyl)phenyl]urea,

N-[(1R,3S)-3-(1-methyl-1H-benzimidazol-2-yl)cyclohexyl]-2,3-dihydro-1,4-benzodioxine-6-carboxamide,

N-{3-[6-(methoxymethyl)-1-methyl-1H-benzimidazol-2-yl]cyclohexyl}-2,3-dihydro-1,4-benzodioxine-6-carboxamide,

3,5-dimethoxy-N-{(1R,3S)-3-[6-(trifluoromethyl)-1H-benzimidazol-2-yl]cyclohexyl}benzamide,

N-[3-(7-methyl-1H-benzimidazol-2-yl )cyclohexyl]-2,3-dihydro-1,4-benzodioxine-6-carboxamide,

N-[3-(6-tert-butyl-1H-benzimidazol-2-yl)cyclohexyl]-2,3-dihydro-1,4-benzodioxine-6-carboxamide,

N-[(3R)-3-(5-methoxy-1H-benzimidazol-2-yl)cyclohexyl]-2,3-dihydro-1,4-benzodioxine-6-carboxamide,

N-[3-(1H-benzimidazol-2-yl)cyclohexyl]-6-methoxy-1-methyl-1H-indole-2-carboxamide,

N-[3-(1H-benzimidazol-2-yl)cyclohexyl]-1H-indole-6-carboxamide,

3,5-dimethoxy-N-{(1R,3S)-3-[6-(trifluoromethyl)-1H-benzimidazol-2-yl]cyclohexyl}benzamide,

N-[(1R,3S)-3-(6-methyl-i H-benzimidazol-2-yl)cyclohexyl]-2,3-dihydro-1,4-benzodioxine-6-carboxamide,

N-[3-(6-chloro-1H-benzimidazol-2-yl)cyclohexyl]-2,3-dihydro-1,4-benzodioxine-6-carboxamide,

N-[3-(5,6-dimethyl-1H-benzimidazol-2-yl)cyclohexyl]-2,3-dihydro-1,4-benzodioxine-6-carboxamide,

1-[(1R,3S)-3-(1H-benzimidazol-2-yl)cyclohexyl]-3-(6-fluoro-4H-1,3-benzodioxin-8-yl)urea,

N-{3-[6-(cyanomethyl)-1-methyl-1H-benzimidazol-2-yl]cyclohexyl}-2,3-dihydro-1,4-benzodioxine-6-carboxamide,

N-[5-(5-chloro-1H-benzimidazol-2-yl)bicyclo[3.1.1]hept-1-yl]-3,5-dimethoxybenzamide,

N-[3-(1H-benzimidazol-2-yl)cyclohexyl]-2-oxo-1,2,3,4-tetrahydroquinoline-6-carboxamide,

N-[(3S)-3-(5-bromo-1H-benzimidazol-2-yl)cyclohexyl]-2,3-dihydro-1,4-benzodioxine-6-carboxamide, and

N-{(1R,3S)-3-[5-(trifluoromethyl)-1H-benzimidazol-2-yl]cyclohexyl}-4,5,6,7,8,9-hexahydro-1H-cycloocta[c]pyrazole-3-carboxamide, or pharmaceutically acceptable salt thereof.

In one preferred embodiment, the compound of Formula I is selected from the group consisting of:

N-((1R,3S)-3-(1H-benzo[d]imidazol-2-yl)cyclohexyl)-2,3-dihydro-[1,4]dioxino[2,3-c]pyridine-7-carboxamide,

1-[(1R,3S)-3-(1H-benzimidazol-2-yl)cyclohexyl]-3-[4-chloro-3-(trifluoromethyl)phenyl]urea,

N-{3-[6-(trifluoromethyl)-1H-benzimidazol-2-yl]cyclohexyl}-2,3-dihydro-1,4-benzodioxine-6-carboxamide,

N-{3-[6-(dimethylamino)-1H-benzimidazol-2-yl]cyclohexyl}-2,3-dihydro-1,4-benzodioxine-6-carboxamide,

N-((3S)-3-(1H-benzo[d]imidazol-2-yl)cyclohexyl)-2,3-dihydrobenzo[b][1,4]dioxine-6-carbothioamide,

N-[(1R,3S)-3-(5-chloro-1H-benzimidazol-2-yl)cyclohexyl]-1-methyl-2-oxo-1,2,3,4-tetrahydroquinoline-6-carboxamide,

4-methyl-3-oxo-N-{(1R,3S)-3-[5-(trifluoromethyl)-1H-benzimidazol-2-yl]cyclohexyl}-3,4-dihydro-2H-1,4-benzoxazine-6-carboxamide,

1-[(1R,3S)-3-(1H-benzimidazol-2-yl)cyclohexyl]-3-(3,5-dichlorophenyl)urea,

N-[(1R,3S)-3-(5-chloro-1H-benzimidazol-2-yl)cyclohexyl]-3,5-dimethoxybenzamide,

N-[(1R 3S )-3-(5-chloro-1H-benzimidazol-2-yl)cyclohexyl]-2-oxo-1,2,3,4-tetrahydroquinoline-6-carboxamide,

1-[(1R, 3S)-3-(1H-benzimidazol-2-yl)cyclohexyl]-3-(3,4-dichlorophenyl)urea,

N-[3-(6-{[(2-methoxyethyl)amino]methyl}-1-methyl-1H-benzimidazol-2-yl)cyclohexyl]-2,3-dihydro-1,4-benzodioxine-6-carboxamide,

1,3-dimethyl-N-{(1R,3S)-3-[5-(trifluoromethyl)-1H-benzimidazol-2-yl]cyclohexyl}-1H-thieno[2,3-c]pyrazole-5-carboxamide,

2-oxo-N-{(1R,3S)-3-[5-(trifluoromethyl)-1H-benzimidazol-2-yl]cyclohexyl}-1,2,3,4-tetrahydroquinoline-6-carboxamide,

4-benzoyl-N-{5-[5-(trifluoromethyl)-1H-benzimidazol-2-yl]bicyclo[3.1.1]hept-1-yl}benzamide,

3,5-dimethoxy-N-((1R,3S)-3-[6-(trifluoromethyl)-1H-benzimidazol-2-yl]cyclohexyl}benzamide,

1-methyl-2-oxo-N-{(1R,3S)-3-[5-(trifluoromethyl)-1H-benzimidazol-2-yl]cyclohexyl}-1,2,3,4-tetrahydroquinoline-6-carboxamide,

N-[3-(6-bromo-1H-benzimidazol-2-yl)cyclohexyl)-2,3-dihydro-1,4-benzodioxine-6-carboxamide,

3,5-dimethoxy-N-{5-[5-(trifluoromethyl)-1H-benzimidazol-2-yl]bicyclo[3.1.1 ]hept-1-yl}benzamide,

1-[(1R,3S)-3-(1H-benzimidazol-2-yl )cyclohexyl]-3-(4-isopropylphenyl)urea,

N-[(1R 3S)-3-(1-methyl-1H-benzimidazol-2-yl)cyclohexyl]-2,3-dihydro-1,4-benzodioxine-6-carboxamide,

N-{5-[5-(trifluoromethyl)-1H-benzimidazol-2-yl]bicyclo[3.1.1]hept-1-yl}-2,3-dihydro-1,4-benzodioxine-6-carboxamide,

N-((1 S,3S)-3-(1H-benzo[d]imidazol-2-yl)cyclohexyl)-3-oxo-3,4-dihydro-2H-benzo[b][1,4]oxazine-6-carboxamide,

N-[3-(6-bromo-1-methyl-1H-benzimidazol-2-yl)cyclohexyl)-3,5-dimethoxybenzamide, and

4-(trifluoroacetyl)-N-{5-[5-(trifluoromethyl)-1H-benzimidazol-2-yl]bicyclo[3.1.1]hept-1-yl}benzamide, or the pharmaceutically acceptable salt thereof.

In another preferred embodiment, the compound of Formula I is selected from the group consisting of:

N-[(1R 3S)-3-(5-chloro-1H-benzimidazol-2-yl)cyclohexyl]-4-methyl-3-oxo-3,4-dihydro-2H-1,4-benzoxazine-6-carboxamide,

1-(4-cyanophenyl)-3-{5-[5-(trifluoromethyl)-1H-benzimidazol-2-yl]bicyclo[3.1.1]hept-1-yl}urea,

3,5-dimethoxy-N-{3-[6-(trifluoromethyl)-1H-benzimidazol-2-yl]cyclohexyl}benzamide,

N-[3-(1H-benzimidazol-2-yl)cyclohexyl]-2-(5-chloro-1H-benzimidazol-2-yl)acetamide,

N-{(3S)-3-[5-(trifluoromethyl)-1H-benzimidazol-2-yl]cyclohexyl}-2,3-dihydro-1,4-benzodioxine-6-carboxamide,

N-[(1R,3S)-3-(6-chloro-1H-benzimidazol-2-yl)cyclohexyl]-2,3-dihydro-1,4-benzodioxine-6-carboxamide,

1-[(1R, 3S)-3-(1H-benzimidazol-2-yl)cyclohexyl]-3-[4-(trifluoromethyl)phenyl urea,

N-[(1R,3S)-3-(1-methyl-1H-benzimidazol-2-yl)cyclohexyl]-2,3-dihydro-1,4-benzodioxine-6-carboxamide,

N-{3-[6-(methoxymethyl)-1-methyl-1H-benzimidazol-2-yl]cyclohexyl}-2,3-dihydro-1,4-benzodioxine-6-carboxamide,

3,5-dimethoxy-N-{(1R,3S)-3-[6-(trifluoromethyl)-1H-benzimidazol-2-yl]cyclohexyl}benzamide,

N-[3-(7-methyl-1H-benzimidazol-2-yl)cyclohexyl]-2 3-dihydro-1,4-benzodioxine-6-carboxamide,

N-[3-(6-tert-butyl-1H-benzimidazol-2-yl)cyclohexyl]-2,3-dihydro-1,4-benzodioxine-6-carboxamide,

N-[(3R)-3-(5-methoxy-1H-benzimidazol-2-yl)cyclohexyl]-2,3-dihydro-1,4-benzodioxine-6-carboxamide,

N-[3-(1H-benzimidazol-2-yl)cyclohexyl]-6-methoxy-1-methyl-1H-indole-2-carboxamide,

N-[3-(1H-benzimidazol-2-yl)cyclohexyl]-1H-indole-6-carboxamide,

3,5-dimethoxy-N-{(1R,3S)-3-[6-(trifluoromethyl)-1H-benzimidazol-2-yl]cyclohexyl}benzamide,

N-[(1R,3S)-3-(6-methyl-1H-benzimidazol-2-yl)cyclohexyl]-2,3-dihydro-1,4-benzodioxine-6-carboxamide,

N-[3-(6-chloro-1H-benzimidazol-2-yl)cyclohexyl]-2,3-dihydro-1,4-benzodioxine-6-carboxamide,

N-[3-(5,6-dimethyl-1H-benzimidazol-2-yl)cyclohexyl]-2,3-dihydro-1,4-benzodioxine-6-carboxamide,

1-[(1R,3S)-3-(1H-benzimidazol-2-yl)cyclohexyl]-3-(6-fluoro-4H-1,3-benzodioxin-8-yl)urea,

N-{3-[6-(cyanomethyl)-1-methyl-1H-benzimidazol-2-yl]cyclohexyl}-2,3-dihydro-1,4-benzodioxine-6-carboxamide,

N-[5-(5-chloro-1H-benzimidazol-2-yl)bicyclo[3.1.1]hept-1-yl]-3,5-dimethoxybenzamide,

N-[3-(1H-benzimidazol-2-yl)cyclohexyl]-2-oxo-1,2,3,4-tetrahydroquinoline-6-carboxamide,

N-[(3S)-3-(5-bromo-1H-benzimidazol-2-yl)cyclohexyl]-2,3-dihydro-1,4-benzodioxine-6-carboxamide, and

N-{(1R,3S )-3-[5-(trifluoromethyl)-1H-benzimidazol-2-yl]cyclohexyl}-4,5,6,7,8,9-hexahydro-1H-cycloocta[c]pyrazole-3-carboxamide, or pharmaceutically acceptable salt thereof.

The present invention also relates to a method for the treatment of abnormal cell growth in a mammal comprising administering to said mammal an amount of a compound of Formula I or pharmaceutically acceptable salt thereof that is effective in treating abnormal cell growth.

In a preferred embodiment the abnormal cell growth is cancer.

In a more preferred embodiment, the cancer is selected from the group consisting of basal cell cancer, medulloblastoma cancer, liver cancer, rhabdomyosarcoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenona, or a combination of one or more of the foregoing cancers.

In another preferred embodiment, the present invention relates to a method for the treatment of cancer solid tumor in a mammal comprising administering to said mammal an amount of a compound of formula I or a pharmaceutical acceptable salt thereof that is effective in treating said cancer solid tumor.

In a more preferred embodiment, the cancer is a solid tumor selected from the group consisting of basal cell cancer, medulloblastoma cancer, liver cancer, rhabdomyosarcoma, lung cancer, bone cancer, and pancreatic cancer.

The present invention also relates to a method for the treatment of abnormal cell growth in a mammal which comprises administering to said mammal an amount of a compound of formula I or a pharmaceutical acceptable salt thereof that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

The present invention also provides for a pharmaceutical composition comprising an amount of a compound of formula I or pharmaceutically acceptable salt thereof, and a pharmaceutically acceptable carrier.

The present invention provides a method for making a compound of formula I, comprising reacting a compound of formula E:

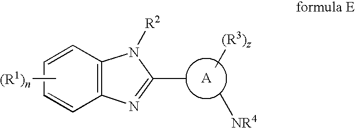

with a compound of formula F:

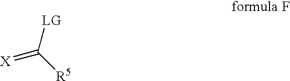

wherein LG is a suitable leaving group.

The present invention also includes isotopically-labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as, but not limited to, ²H, ³H, ¹³C, ¹⁴C, ¹⁵N, ¹⁸O, 17O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as 3H and 14C are incorporated, are useful in drug and/or substrate tissue distribution assays. Tritiated, i.e., 3H, and carbon-14, i.e., 14C, isotopes are particularly preferred for their ease of preparation and detectability. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically-labelled compounds of this invention and prodrugs thereof can generally be prepared by carrying out the procedures disclosed in the Schemes and/or in the Examples and Preparations below, by substituting a readily available isotopically-labelled reagent for a non-isotopically-labelled reagent.

The present invention also relates to the pharmaceutically acceptable acid addition salts of the compounds of the invention. The acids which are used to prepare the pharmaceutically acceptable acid addition salts of the aforementioned base compounds of this invention are those which form non toxic acid addition salts, i.e., salts containing pharmacologically acceptable anions, such as, but not limited to, the chloride, bromide, iodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p toluenesulfonate and pamoate [i.e., 1,1′ methylene bis (2 hydroxy 3 naphthoate)]salts.

The invention also relates to base addition salts of the compounds of the invention. The chemical bases that may be used as reagents to prepare pharmaceutically acceptable base salts of those compounds of the compounds of the invention that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

As used herein, the phrase “compound of formula I” includes prodrugs, solvates or hydrates thereof.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

This invention also encompasses pharmaceutical compositions containing prodrugs of compounds of the the compounds of the invention. Compounds of the compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of the invention. The amino acid residues include the 20 naturally occurring amino acids commonly designated by three letter symbols and also include, 4 hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Prodrugs also include compounds wherein carbonates, carbamates, amides and alkyl esters that are covalently bonded to the above substituents of the compounds of the invention through the carbonyl carbon prodrug sidechain.

This invention also encompasses compounds of the invention containing protective groups. One skilled in the art will also appreciate that compounds of the invention can also be prepared with certain protecting groups that are useful for purification or storage and can be removed before administration to a patient. The protection and deprotection of functional groups is described in “Protective Groups in Organic Chemistry”, edited by J. W. F. McOmie, Plenum Press (1973) and “Protective Groups in Organic Synthesis”, 3rd edition, T. W. Greene and P. G, M. Wuts, Wiley-Interscience (1999).

The compounds of this invention include all stereoisomers (e.g., cis and trans isomers) and all optical isomers of compounds of the the invention (e.g., R and S enantiomers), as well as racemic, diastereomeric and other mixtures of such isomers. While all stereoisomers are encompassed within the scope of our claims, one skilled in the art will recognize that particularly stereoisomers may be preferred. For example, in the case where A is a cyclohexane ring, preferred compounds contain the R configuration at the point of attachment to the —N(R⁴)C(═X)R⁵ moiety as shown below in the structure (i). The most preferred compounds when A is a cyclohexane ring have the R configuration at the point of attachment to the —N(R⁴)C(═X)R⁵ moiety and the S configuration at point of attachment to the benzimidazole moiety as shown below in the structure (ii). The particular preferred stereochemistry for other A groups is determined on a case by case

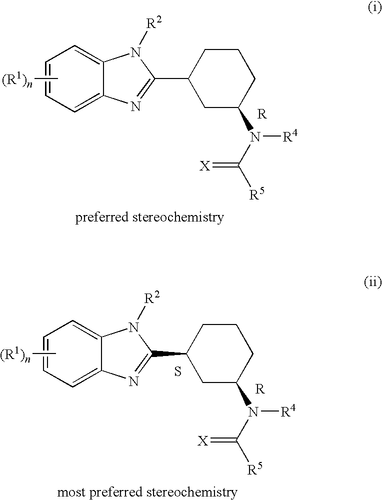

The compounds, salts and prodrugs of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds.

The present invention also includes atropisomers of the present invention. Atropisomers refer to compounds of the invention that can be separated into rotationally restricted isomers.

The term “alkyl”, as used herein means one to ten, preferably one to six, saturated monovalent hydrocarbon radicals having straight or branched moieties.

The terms “carbocycle”, “carbocyclyl”, “carbocyclo”, or “carbocyclic” as used herein means an aliphatic ring system having three to twelve members. The terms “carbocycle”, “carbocyclyl”, “carbocyclo”, or “carbocyclic” whether saturated or partially unsaturated, also refers to rings that are optionally substituted. The terms “carbocycle”, “carbocyclyl”, “carbocyclo”, or “carbocyclic” also include aliphatic rings that are fused to one or more aromatic or non-aromatic rings, such as in a decahydronaphthyl or tetrahydronaphthyl, where the radical or point-of attachment is on the aliphatic ring.

As used herein, the term “cycloalkyl” refers to a mono, fused or bridged bicyclic or tricyclic carbocyclic rings, (e.g., cyclopropyl, cyclobutyl, cyclopentyl, cyclohexyl, cycloheptyl, cyclopentenyl, cyclohexenyl, bicyclo[2.2.1]heptanyl, bicyclo[3.2.1]octanyl and bicyclo[5.2.0]nonanyl, norbornyl, adamantanyl, etc.); said rings may optionally containing 1 or 2 double bonds. The term “cycloalkyl” also includes spiro cycloalkyl groups, including, without limitation multi-ring systems joined by a single atom.

The term “1,3-” as applied to cycloalkyl in the context of A refers to the relative points of attachment of the —N(R⁴)C(═X)R⁵ and benzimidazole moieties. Thus, said moieties are attached to carbon atoms of A that are separated from each other in some instance within the structure of A by a single carbon atom. The term “1,3-” as applied to cycloalkyl in the context of A does not necessarily refer to conventional numbering nomenclature of said cycloalkyl groups although it may in fact do so. For example, when A is cyclohexane the term “1,3-” would coincide with conventional numbering. However, in another example when A is bicyclo[3.1.1]heptane conventional numbering would not necessarily indicate the points of attachment of the —N(R⁴)C(═X)R⁵ and benzimidazole moieties but rather refers to their relative positions on the bicycle[3.1.1]heptane ring system.

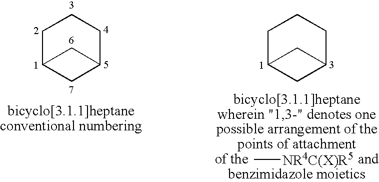

In a further example, when A is a spiro[2.5]octane the conventional numbering would not necessarily indicate the points of attachment of the —N(R⁴)C(═X)R⁵ and benzimidazole moieties but rather refers to their relative positions on the spiro[2.5]octane system.

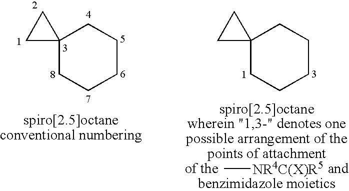

In a further example, when A is cyclopropyl, the conventional numbering would not necessarily indicate the points of attachment of the —N(R⁴)C(═X)R⁵ and benzimidazole moieties but rather refers to their relative positions on the cyclopropyl ring system as shown below.

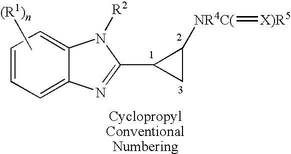

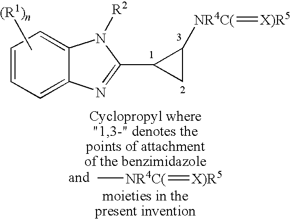

The term “alkoxy”, as used herein means O-alkyl groups wherein alkyl is as defined above.

The terms “hydroxyalkyl”, “alkoxyalkyl”, and alkoxycarbonyl“, used alone or as part of a larger moiety includes both straight and branched chains containing one to six carbon atoms.

The term “alkenyl” used alone or as part of a larger moiety shall include both straight and branched chains containing two to ten carbon atoms having at least one carbon-carbon double bond. The terms “alkynyl” used alone or as part of a larger moiety shall include both straight and branched chains containing two to ten carbon atoms having at least one carbon-carbon triple bond.

The terms “haloalkyl”, “haloalkenyl” and haloalkoxy” means alkyl, alkenyl or alkoxy, as the case may be, substituted with one or more halogen atoms. The term “halo” is used herein interchangeably with the term “halogen” means F, Cl, Br, or I. Preferred halo groups are F, Cl, and Br.

The term “heteroatom”, means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0 to 3 heteroatoms selected from oxygen, sulfur or nitrogen, the nitrogen may be N (as in 3,4-dihydro-2H-pyrrolyl), NH (as in pyrrolidinyl) or NOR (as in N-substituted pyrrolidinyl).

The term “aryl” may be used interchangeably with the term aryl ring. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl” as it is used herein, is a group in which an aromatic ring is fused to one or more non-aromatic rings, such as in an indanyl, phenanthridinyl, or tetrahydronaphthyl, where the radical or point of attachment is on the aromatic ring. The term “aryl” also refers to rings that are optionally substituted.

The term “heterocycle”, “heterocyclyl”, or “heterocyclic” as used herein includes aromatic and non-aromatic ring systems having four to fourteen members, preferably five to ten, in which one or more ring carbons, preferably one to four, are each replaced by a heteroatom such as N, O, or S. Non-aromatic heterocyclic groups include groups having only 4 atoms in their ring system, but aromatic heterocyclic groups must have at least 5 atoms in their ring system. The heterocyclic groups include benzo-fused ring systems. Examples of heterocyclic rings include 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzo[1,3]dioxine, benzo[1,4]dioxine, benzopyrrolidinyl, benzopiperidinyl, benzoxolanyl, benzothiolanyl, 4,5,6,7-tetrahydropyrazol[1,5-alpha]pyridine and benzothianyl.

Also included within the scope of the term “heterocyclyl”, or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring.

The term “heterocycle”, “heterocyclyl”, or “heterocyclic” whether saturated or partially unsaturated, also refers to rings that are optionally substituted.

An example of a 4 membered heterocyclic group is azetidinyl (derived from azetidine). An example of a 5 membered heterocyclic group is thiazolyl and an example of a 10 membered heterocyclic group is quinolinyl.

Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl.

Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-2-yl (C-attached).

An example of a heterocyclic group wherein 1 or 2 ring carbon atoms are substituted with oxo (═O) moieties is 1,1-dioxo-thiomorpholinyl, thienopyridinone, or pyrimidine-2,4-dione. An example of a heterocyclic group wherein 1 ring sulfur atom is substituted with 2 oxo (═O) moieties is tetrahydrothiophenedioxide.

Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquinolinyl, and pyrido[3,4-dlpyrimidinyl.

The term “heteroaryl”, used alone or as part of a larger moiety as in “heteroaralkyl” or “heteroarylalkoxy”, refers to heteroaromatic ring groups having five to fourteen members. Examples of heteroaryl rings include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2 5 pyrazolyl, 3-pyrazolyl, 2-pyridyl, 3-pyridylj 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, or benzoisoxazolyl.

The term “heteroaryl” also refers to rings that are optionally substituted. The term “heteroaryl” may be used interchangeably with the term “heteroaryl ring” or the term “heteroaromatic”. An aryl (including aralkyl, aralkoxy, aryloxyalkyl and the like) or heteroaryl (including heteroaralkyl and heteroarylalkoxy and the like) group may contain one or more R⁵ substituents.

When preparing compounds of the invention in accordance with the invention, it is open to a person skilled in the art to routinely select the form of the intermediate compound which provides the best combination of features for this purpose. Such features include the melting point, solubility, processability and yield of the intermediate form and the resulting ease with which the product may be purified on isolation.

The invention also relates to methods for making intermediate compounds that are useful for making the compounds of the invention.

As noted above, invention also relates to the pharmaceutically acceptable salts of the compounds of the invention. Pharmaceutically acceptable salts of the compounds of the invention include the acid addition and base salts thereof. Suitable acid addition salts are formed from acids which form non-toxic salts. Non-limiting examples of suitable acid addition salts include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloridelchloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Non-limiting examples of suitable base salts include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art.

The compounds of the invention may also exist in unsolvated and solvated forms. Accordingly, the invention also relates to the hydrates and solvates of the compounds of the invention.

The term “solvate” is used herein to describe a molecular complex comprising the compound of the invention and one or more pharmaceutically acceptable solvent molecules, for example, ethanol.

The term ‘hydrate’ is employed when said solvent is water. A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The invention also relates to prodrugs of the compounds of the invention. Thus certain derivatives of compounds of the invention which may have little or no pharmacological activity themselves can, when administered into or onto the body, be converted into compounds of the invention having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of the invention with certain moieties known to those skilled in the art as ‘pro-moieties’ as described, for example, in Design of Prodrugs by H. Bundgaard (Elsevier, 1985).

Some non-limiting examples of prodrugs in accordance with the invention include

(i) where the compound of the invention contains a carboxylic acid functionality

(—COOH), an ester thereof, for example, a compound wherein the hydrogen of the carboxylic acid functionality of the compound of formula I is replaced by (C₁-C₆)alkyl;

(ii) where the compound of the invention contains an alcohol functionality (—OH), an ether thereof, for example, a compound wherein the hydrogen of the alcohol functionality of the compound of the invention is replaced by (C₁-C₆)alkanoyloxymethyl; and

(iii) where the compound of the invention contains a primary or secondary amino functionality (—NH₂ or —NHR where R≠H), an amide thereof, for example, a compound wherein, as the case may be, one or both hydrogens of the amino functionality of the compound of the invention is/are replaced by (C₁-C₆)alkanoyl.

Further examples of replacement groups in accordance with the foregoing examples and examples of other prodrug types may be found in the aforementioned references.

Moreover, certain compounds of the invention may themselves act as prodrugs of other compounds of the invention.

Also included within the scope of the invention are metabolites of compounds of the invention, that is, compounds formed in vivo upon administration of the drug. Some examples of metabolites in accordance with the invention include:

(i) where the compound of the invention contains a methyl group, an hydroxymethyl derivative thereof (e.g., —CH₃→—CH₂OH):

(ii) where the compound of the invention contains an alkoxy group, an hydroxy derivative thereof (e.g., —OH);

(iii) where the compound of the invention contains a tertiary amino group, a secondary amino derivative thereof;

(iv) where the compound of the invention contains a secondary amino group, a primary derivative thereof (e.g., —NH₂);

(v) where the compound of the invention contains a phenyl moiety, a phenol derivative thereof (e.g., -Ph→-PhOH); and

(vi) where the compound of the invention contains an amide group, a carboxylic acid derivative thereof (e.g., —CONH₂→COOH).

Compounds of the invention containing one or more asymmetric carbon atoms can exist as two or more stereoisomers. Where a compound of the invention contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counterion is optically active, for example, d-lactate or I-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of an alcoholic solvent such as isopropanol, typically from 2% to 20%, and from 0 to 5% by volume of an alkylamine, typically 0.1% diethylamine. Concentration of the eluate affords the enriched mixture.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The invention also relates to methods for the treartment of abnormal cell growth in a mammal. In one embodiment the invention relates to a method for the treatment of abnormal cell growth in a mammal comprising administering to said mammal an amount of a compound of the invention that is effective in treating abnormal cell growth.

In another embodiment the abnormal cell growth is cancer.

In another embodiment the cancer is selected from the group consisting of lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

The invention also relates to methods for the treatment of cancer solid tumors in a mammal. In one embodiment the invention relates to the treatment of cancer solid tumor in a mammal comprising administering to said mammal an amount of a compound of the invention that is effective in treating said cancer solid tumor.

In another embodiment the cancer solid tumor is breast, lung, colon, brain, prostate, stomach, pancreatic, ovarian, skin (melanoma), endocrine, uterine, testicular, or bladder.

In another embodiment the invention relates to a method for the treatment of abnormal cell growth in a mammal which comprises administering to said mammal an amount of a compound of the invention that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In still another embodiment the invention relates to a pharmaceutical composition for the treatment of abnormal cell growth in a mammal comprising an amount of a compound of the invention that is effective in treating abnormal cell growth, and a pharmaceutically acceptable carrier.

### DETAILED DESCRIPTION OF THE INVENTION

The compounds of the invention can be prepared by the following general methods and by methods described in detail in the Experimental Section.

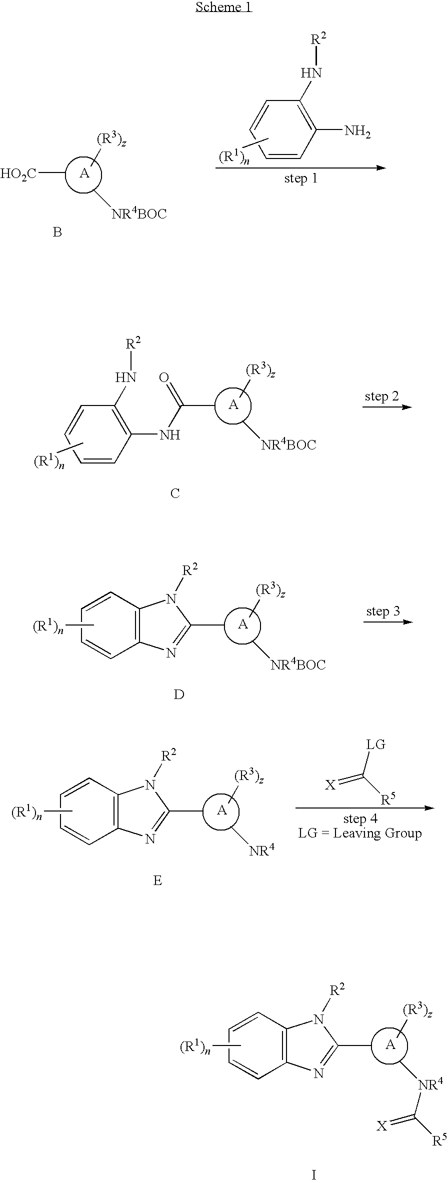

Compounds claimed herein can be prepared as described in Scheme 1 wherein in Step 1 a compound of Formula B, substituted with a carboxylic acid and a protected amine, is reacted with a substituted benzene-1,2-diamine in the presence of a coupling reagent such as N-(3-dimethylaminopropyl)-N′-ethylcarbodiimide hydrochloride, propylphosphonic acid anhydride, or other amide forming reagents well known to those skilled in the art. Starting materials are commercially available, unless otherwise noted in the Examples. In the following examples and preparations, “BOC”, “Boc” or “boc” means N-tert-butoxycarbonyl, “DCM” (CH₂Cl₂) means methylene chloride, “DIPEA” or “DIEA” means diisopropyl ethyl amine, “DMA” means N,N-dimethylacetamide, “DMF” means N-N-dimethyl formamide, “DMSO” means dimethylsulfoxide, “DPPP” means 1,3-bis(diphenylphosphino)propane, “HOAc” means acetic acid, “IPA” means isopropyl alcohol. “MTBE” means methyl t-butyl ether, “NMP” means 1-methyl 2-pyrrolidinone, “TEA” means triethyl amine, “TFA” means trifluoroacetic acid, “THF” means tetrahydrofuran, “DCM” means dichloromethane, “EtOAc” means ethyl acetate, “MgSO₄” means magnesium sulphate, “NaSO4” means sodium sulphate, “MeOH” means methanol, “EtOH” means ethanol, “H₂O” means water, “HCl” means hydrochloric acid, “POCl₃” means phosphorus oxychloride, “DMSO” means dimethyl sulfoxide, and “K₂CO₃” means potassium carbonate. Note that A in a circle represents a 1,3-cycloalkyl as defined herein. The reaction is best performed in an aprotic solvent such as tetrahydrofuran, 1,4-dioxane, dimethylformamide, or acetonitrile. The reaction can be performed at a range of temperatures but generally from room temperature to 80 □C. In Step 2 the resulting benzene-1,2-diamine mono amide can then be cyclized to form the benzimidazole ring by heating to about 100 □C in the presence of an acid such as acetic acid, or by treatment with additional portions of a coupling agent as described above. Subsequently in Step 3 the t-butoxycarbonyl protecting group is removed with a suitable acid such as hydrogen chloride in an appropriate solvent such as 1,4-dioxane, ethyl acetate or methylene chloride or with trifluoroacetic acid, neat or in an appropriate solvent such as methylene chloride. Step 3 can be performed at a range of temperatures but generally from 0 □C to room temperature. One skilled in the art will recognize that although a t-butoxycarbonyl protecting group is shown in FIG. 1, the amine could be protected in alternate ways, such as with a benzyloxycarbonyl or phthalimido group, each of which would be removed by methods known to one skilled in the art. In Step 4 the amino group attached to compound B can be reacted with, for example, an activated carboxylic acid, isocyanate or carbamoyl chloride to produce the compounds of formula I claimed herein. The carboxylic acid may be activated as a carboxylic acid chloride, as a mixed anhydride, formed from, for example pivaloyl chloride or isopropylchloroformate, or as an active intermediate such as is formed by treatment of a carboxylic acid with coupling reagents such as N-(3-dimethylaminopropyl)-N′-ethylcarbodiimide hydrochloride, propylphosphonic acid anhydride, or other amide forming reagents well known to those skilled in the art. Step 4 is best performed in an aprotic solvent such as tetrahydrofuran, 1,4-dioxane, or dimethylformamide and at a range of temperatures but generally from room temperature to 80 □C.

One skilled in the art will also recognize that the compound of formula B could alternatively be substituted with a free amine and a protected carboxylic acid, protected, for example, as a methyl, ethyl, benzyl or t-butyl ester. In such a case, the free amino group would be elaborated to form the —NR⁴C(═X)R⁵ group as described above for Step 4 which would be followed by deprotection of the carboxylic acid by means known to one skilled in the art. For example, an ethyl ester could be saponified with lithium hydroxide, sodium hydroxide or potassium hydroxide in, for example, an alcoholic solvent such as ethanol or in a mixture of an organic solvent such as ethanol, methanol or tetrahydrofuran with water. The saponification could be performed at a range of temperatures but generally at from room temperature to about 80 □C. The resulting carboxylic acid could be converted into the requisite benzimidazole by the procedures described above for Steps 1 and 2.

All of the reactions described above can be performed for a range of times from a few minutes to a few days but generally from 1 hour to 24 hours.

As noted above, the compounds of the invention are useful as inhibitors of SMO. Methods for determining the in vitro activity of these compounds are described below: Smoothened (SMO)/Sonic Hedgehog (SHh) Transient Transcriptional Activation Assay

On Day 1,2×106 C3H10T1/2 cells (ATCC #CCL-226) were split and seeded in 12 mls of growth medium Basal Medium Eagle (BME, Invitrogen #21010-046) supplemented with 2 mM L-glutamine (Invitrogen #25030-081), 0.1 units/ml penicillin and 0.1 ug/ml streptomycin (Invitrogen #15140-122), and 10% Fetal Bovine Serum (FBS, Invitrogen #16140-071) in a T-75 flask (Costar #3376). They were allowed to attach for 4 hours at 37° C., 5% CO2. The cells were then transfected using Fugene 6 (Roch #11 814 443 001) in the following reaction: 48 ul Fugene 6 and 745 ul Opti-MEM (Invitrogen #31985-070) were mixed and allowed to sit at room temperature for 5 minutes. 8 ug of pGL4.14/mGli(CS) DNA (10× murine Gli response elements and minimal CS promoter) and 0.5 ug of pEGFP DNA (Clontech) were added, gently mixed and incubated at room temperature for 20 minutes. This entire transfection mix was then added to the T-75 flask containing the cells. The cells were incubated at 37 □C, 5% C0₂ for 18-24 hrs.

On Day 2, the transfected cells were trypsinized and seeded into white 96 well plates (Costar #3917) in 100ul/well of growth medium at a concentration of 20,000 cells/well. The cells were allowed to recover for 4 hrs before adding serum starvation medium Dulbecco's Modified Eagle Medium (DMEM, Invitrogen #21063-029) supplemented with 2 mM L-glutamine, 0.1 units/ml penicillin and 0.1 ug/ml streptomycin, and 0.5% Calf Serum (CS, Invitrogen #26170-043). The growth media was aspirated off, and the cells were rinsed with 100 ul of starvation media. 95 ul of starvation media was then added to each well. The cells were incubated for 20 hrs at 37 □C, 5% C0₂.

On Day 3, cells were dosed with test compounds at a final concentration ranging from 2 uM to 2 nM. Immediately after dosing cells with compounds, recombinant human sonic hedgehog (SHh, R&D Systems #1845-SH) was add to a final concentration of 250 ng/ml. A 25 ug vial of SHh was reconstituted with 250 ul PBS/0.1% BSA to give a 100 ng/ul working stock. This working stock was then diluted 1:20 in starvation media. The transfected cells were incubated with compounds and SHh for 20 hrs at 37 □C, 5% C0₂.

Luciferase assays were conducted on Day 4 using Dual-Glo Luciferase assay system (Promega #E2940) according to Promega's protocol. Briefly, Dual-Glo luciferase reagent was made up and 100 uls were added to each well of the 96 well plate containing media. Plates were shaken at room temperature for 10 minutes, and then read on TopCount (Perkin-Elmer). The luminescence was recorded.

This invention also relates to a method for the treatment of abnormal cell growth in a mammal, including a human, comprising administering to said mammal an amount of a compound of the the invention, as defined above, or a pharmaceutically acceptable salt, solvate, hydrate or prodrug thereof, that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In one embodiment the method comprises comprising administering to a mammal an amount of a compound of the invention that is effective in treating said cancer solid tumor. In one preferred embodiment the solid tumor is breast, lung, colon, brain, prostate, stomach, pancreatic, ovarian, skin (melanoma), endocrine, uterine, testicular, and bladder cancer.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of the invention, or a pharmaceutically acceptable salt, solvate, hydrate or prodrug thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a compound of the invention, as defined above, or a pharmaceutically acceptable salt, solvate, hydrate or prodrug thereof, that is effective in treating abnormal cell growth, and a pharmaceutically acceptable carrier. In one embodiment of said composition, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of the invention, or a pharmaceutically acceptable salt, solvate, hydrate or prodrug thereof, that is effective in treating abnormal cell growth in combination with another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens. The invention also contemplates a pharmaceutical composition for treating abnormal cell growth wherein the composition includes a compound of the invention, as defined above, or a pharmaceutically acceptable salt, solvate, hydrate or prodrug thereof, that is effective in treating abnormal cell growth, and another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a method for the treatment of a disorder associated with angiogenesis in a mammal, including a human, comprising administering to said mammal an amount of a compound of the invention, as defined above, or a pharmaceutically acceptable salt, solvate, hydrate or prodrug thereof, that is effective in treating said disorder in combination with one or more anti-tumor agents listed above. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of a compound of the invention, or a pharmaceutically acceptable salt, solvate, hydrate or prodrug thereof, in combination with an amount of one or more substances selected from anti-angiogenesis agents, signal transduction inhibitors, and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of the invention in the methods and pharmaceutical compositions described herein. Examples of useful COX-II inhibitors include CELEBREX™ (celecoxib), Bextra (valdecoxib), paracoxib, Vioxx (rofecoxib), and Arcoxia (etoricoxib). Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340,RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R, 3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hyd roxycarbamoyl-cyclobutyl)-amino]-propionic acid;

4-[4-(4-chloro-phenoxy)-benzenesulfonylamino]-tetrahydro-pyran-4-carboxylic acid hydroxyamide;

3-[4-(4-chloro-phenoxy)-benzenesulfonylamino]-tetrahydro-pyran-3-carboxylic acid hydroxyamide;

(2R, 3R) 1-[4-(4-fluoro-2-methyl-benzyloxy)-benzenesulfonyl]-3-hydroxy-3-methyl-piperidine-2-carboxylic acid hydroxyamide;

3-([4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1]octane-3-carboxylic acid hydroxyamide; and

3-[4-(4-fluora-phenoxy)-benzenesulfanylamino]-tetrahydro-furan-3-carboxylic acid hydroxyamide;

and pharmaceutically acceptable salts, solvates and prodrugs of said compounds.

VEGF inhibitors, for example, SU-11248, SU-5416 and SU-6668 (Sugen Inc. of South San Francisco, Calif., USA), can also be combined with a compound of the invention. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); Avastin, an anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome pic), and the monoclonal antibodies AR-209 (Aronex Pharmaceuticals Inc. of The Woodlands, Tex., USA) and 2B-1 (Chiron), may be administered in combination with a compound of the invention. Such erbB2 inhibitors include Herceptin, 2C4, and pertuzumab. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety. Other erbb2 receptor inhibitors include TAK-165 (Takeda) and GW-572016 (Glaxo-Wellcome).

Various other compounds, such as styrene derivatives, have also been shown to possess tyrosine kinase inhibitory properties, and some of tyrosine kinase inhibitors have been identified as erbB2 receptor inhibitors. More recently, five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent applications Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following U.S. provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

A compound of the invention may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Provisional Application 60/113,647 (filed Dec. 23, 1998) which is herein incorporated by reference in its entirety.

A compound of the invention may be applied as a sole therapy or may involve one or more other anti-tumor substances, for example those selected from, for example, mitotic inhibitors, for example vinblastine; alkylating agents, for example cis-platin, oxaliplatin, carboplatin and cyclophosphamide; anti-metabolites, for example 5-fluorouracil, capecitabine, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex (tamoxifen) or, for example anti-androgens such as Casodex (4′-cyano-3-(4-fluorophenylsulphonyl)-2-hydroxy-2-methyl-3′-(trifluoromethyl)propionanilide).

The compounds of the present invention may be used alone or in combination with one or more of a variety of anti-cancer agents or supportive care agents. For example, the compounds of the present invention may be used with cytotoxic agents, e.g., one or more selected from the group consisting of a camptothecin, irinotecan HCl (Camptosar), edotecarin, SU-11248, epirubicin (Ellence), docetaxel (Taxotere), paclitaxel, rituximab (Rituxan) bevacizumab (Avastin), imatinib mesylate (Gleevac), Erbitux, gefitinib (Iressa), and combinations thereof. The invention also contemplates the use of the compounds of the present invention together with hormonal therapy, e.g., exemestane (Aromasin), Lupron, anastrozole (Arimidex), tamoxifen citrate (Nolvadex), Trelstar, and combinations thereof. Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

The compounds of the invention may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the compounds of the invention.

Alkylating agents include, but are not limited to, nitrogen mustard N-oxide, cyclophosphamide, ifosfamide, melphalan, busulfan, mitobronitol, carboquone, thiotepa, ranimustine, nimustine, temozolomide, AMD-473, altretamine, AP-5280, apaziquone, brostallicin, bendamustine, carmustine, estramustine, fotemustine, glufosfamide, ifosfamide, KW-2170, mafosfamide, and mitolactol; platinum-coordinated alkylating compounds include but are not limited to, cisplatin, carboplatin, eptaplatin, lobaplatin, nedaplatin, oxaliplatin or satrplatin;

Antimetabolites include but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, gemcitabine, fludarabin, 5-azacitidine, capecitabine, cladribine, clofarabine, decitabine, eflornithine, ethynylcytidine, cytosine arabinoside, hydroxyurea, TS-1, melphalan, nelarabine, nolatrexed, ocfosfate, disodium premetrexed, pentostatin, pelitrexol, raltitrexed, triapine, trimetrexate, vidarabine, vincristine, vinorelbine; or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid;

Antibiotics include but are not limited to: aclarubicin, actinomycin D, amrubicin, annamycin, bleomycin, daunorubicin, doxorubicin, elsamitrucin, epirubicin, galarubicin, idarubicin, mitomycin C, nemorubicin, neocarzinostatin, peplomycin, pirarubicin, rebeccamycin, stimalamer, streptozocin, valrubicin or zinostatin;

Hormonal therapy agents, e.g., exemestane (Aromasin), Lupron, anastrozole (Arimidex), doxercalciferol, fadrozole, formestane, anti-estrogens such as tamoxifen citrate (Nolvadex) and fulvestrant, Trelstar, toremifene, raloxifene, lasofoxifene, letrozole (Femara), or anti-androgens such as bicalutamide, flutamide, mifepristone, nilutamide, Casodex® (4′-cyano-3-(4-fluorophenylsulphonyl)-2-hydroxy-2-methyl-3′-(trifluoromethyl)propionanilide) and combinations thereof;

Plant derived anti-tumor substances include for example those selected from mitotic inhibitors, for example vinblastine, docetaxel (Taxotere) and paclitaxel;

Cytotoxic topoisomerase inhibiting agents include one or more agents selected from the group consisting of aclarubicn, amonafide, belotecan, camptothecin, 10-hydroxycamptothecin, 9-aminocamptothecin, diflomotecan, irinotecan HCl (Camptosar), edotecarin, epirubicin (Ellence), etoposide, exatecan, gimatecan, lurtotecan, mitoxantrone, pirarubicin, pixantrone, rubitecan, sobuzoxane, SN-38, tafluposide, and topotecan, and combinations thereof;

Immunologicals include interferons and numerous other immune enhancing agents. Interferons include interferon alpha, interferon alpha-2a, interferon, alpha-2b, interferon beta, interferon gamma-la or interferon gamma-n1. Other agents include PF3512676, filgrastim, lentinan, sizofilan, TheraCys, ubenimex, WF-10, aldesleukin, alemtuzumab, BAM-002, dacarbazine, daclizumab, denileukin, gemtuzumab ozogamicin, ibritumomab, imiquimod, lenograstim, lentinan, melanoma vaccine (Corixa), molgramostim, OncoVAX-CL, sargramostim, tasonermin, tecleukin, thymalasin, tositumomab, Virulizin, Z-100, epratuzumab, mitumomab, oregovomab, pemtumomab, Provenge;

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, or ubenimex;

Other anticancer agents include alitretinoin, ampligen, atrasentan bexarotene, bortezomib. Bosentan, calcitriol, exisulind, finasteride,fotemustine, ibandronic acid, miltefosine, mitoxantrone, I-asparaginase, procarbazine, dacarbazine, hydroxycarbamide, pegaspargase, pentostatin, tazarotne, TLK-286, Velcade, Tarceva, or tretinoin;

Other anti-angiogenic compounds include acitretin, fenretinide, thalidomide, zoledronic acid, angiostatin, aplidine, cilengtide, combretastatin A-4, endostatin, halofuginone, rebimastat, removab, Revlimid, squalamine, ukrain and Vitaxin;

Platinum-coordinated compounds include but are not limited to, cisplatin, carboplatin, nedaplatin, or oxaliplatin;

Camptothecin derivatives include but are not limited to camptothecin, 10-hydroxycamptothecin, 9-aminocamptothecin, irinotecan, SN-38, edotecarin, and topotecan;

Tyrosine kinase inhibitors are Iressa or SU5416;

Antibodies include Herceptin, Erbitux, Avastin, or Rituximab;

Interferons include interferon alpha, interferon alpha-2a, interferon, alpha-2b, interferon beta, interferon gamma-la or interferon gamma-n1;

Biological response modifiers are agents that modify defense mechanisms of living organisms or biological responses, such as survival, growth, or differentiation of tissue cells to direct them to have anti-tumor activity. Such agents include krestin, lentinan, sizofiran, picibanil, or ubenimex; and

Other antitumor agents include mitoxantrone, I-asparaginase, procarbazine, dacarbazine, hydroxycarbamide, pentostatin, or tretinoin. “Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The compounds of the present invention are potent inhibitors of SMO, and thus are all adapted to therapeutic use as antiproliferative agents (e.g., anticancer), antitumor (e.g., effective against solid tumors), antiangiogenesis (e.g., stop or prevent proliferationation of blood vessels) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH). It is, in addition, expected that a compound of the present invention may possess activity against a range of leukemias and lymphoid malignancies.

In one embodiment of the present invention cancer is selected from lung cancer, bone cancer, pancreatic cancer, gastric, skin cancer, cancer of the head or neck, cutaneous or intraocular melanoma, uterine cancer, ovarian cancer, gynecological, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, squamous cell, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain, pituitary adenoma, or a combination of one or more of the foregoing cancers.

In another embodiment cancer is selected a solid tumor, such as, but not limited to, breast, lung, colon, brain (e.g., glioblastoma), prostate, stomach, pancreatic, ovarian, skin (melanoma), endocrine, uterine, testicular, and bladder.

The methods of the present invention include the use of small molecules which inhibit Smo, in the regulation of repair and/or functional performance of a wide range of cells, tissues and organs, including normal cells, tissues, and organs, as well as those having the phenotype of ptc loss-of-function, hedgehog gain-of-function, or smoothened gain-of-function. For instance, the subject method has therapeutic and cosmetic applications ranging from regulation of neural tissues, bone and cartilage formation and repair, regulation of spermatogenesis, regulation of smooth muscle, regulation of lung, liver and other organs arising from the primative gut, regulation of hematopoietic function, regulation of skin and hair growth, etc. Moreover, the subject methods can be performed on cells that are provided in culture (in vitro), or on cells in a whole animal (in vivo). See, for example, PCT publications WO 95/18856 and WO 96/17924.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The present invention also provides a pharmaceutical composition comprising a compound of formula I, or a pharmaceutically acceptable salt or solvate thereof, as hereinbefore defined in association with a pharmaceutically acceptable adjuvant, diluent or carrier.

The invention further relates to a pharmaceutical composition of the invention which comprises mixing a compound of formula I, or a pharmaceutically acceptable salt or solvate thereof, as hereinbefore defined with a pharmaceutically acceptable adjuvant, diluent or carrier.

For the above-mentioned therapeutic uses the dosage administered will, of course, vary with the compound employed, the mode of administration, the treatment desired and the disorder indicated. The daily dosage of the compound of formula I or pharmaceutically acceptable salt may be in the range from 1 mg to I gram, preferably 1 mg to 250 mg, more preferably 10 mg to 100 mg.

The present invention also encompasses sustained release compositions.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex® (tamoxifen) or, for example anti-androgens such as Casodex® (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### Examples

Where HPLC chromatography is referred to in the preparations and examples below, the general conditions used, unless otherwise indicated, are as follows. The column used is a Polaris 5 C18-A column, 20×2.0 mm, with a 3.76 minute gradient elution starting at 95% A/5% B (A: 98% water, 2% acetonitrile, 0.01% formic acid; B: 100% acetonitrile, 0.005% formic acid) ending at 100% B with a 1.0 mL/min flow rate. Compounds were detected by UV absorption and electrospray mass ionization.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### Example 1

### N-(3-(1H-benzo[d]midazol-2-yl)cyclohexyl)-2,3-dihydrobenzo[b][1,4]dioxine-6-carboxamide

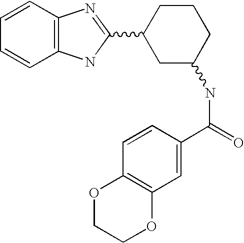

3-(tert-Butoxycarbonyl)cyclohexanecarboxylic acid. A mixture of 3-aminocyclohexanecarboxylic acid (25 g, 175 mmol), di-tert-butyl dicarbonate (49.5 g, 227 mmol), diisopropylethylamine (34 ml, 193 mmol), THF (100 ml), and water (100 ml) was stirred at room temperature for 3 hours. The reaction mixture was concentrated to about one half of the initial volume and 35 ml of 6 M hydrochloric acid was added. The resulting mixture was extracted with 300 ml of MTBE. The organic extract was dried over anhydrous magnesium sulfate, concentrated in vacuum and dried in high vacuum at 45° C. to provide the desired product was as a white solid (41.4 g, 97%).

3-(1H-Benzo[d]imidazol-2-yl)cyclohexanamine dihydrochloride. A mixture of 3-(tert-butoxycarbonyl)cyclohexane-carboxylic acid (15.3 g, 62.9 mmol), benzene-1,2-diamine (6.8 g, 62.9 mmol), N-(3-dimethylaminopropyl)-N′-ethylcarbodiimide hydrochloride (14.5 g, 75.5 mmol), dimethylaminopyridine (400 mg, 3.2 mmol), and DMF (200 ml) was stirred at room temperature for 18 hours. Most of DMF was removed in vacuum and the resulting mixture was partitioned between 500 ml of 3% aqueous sodium bicarbonate and 400 ml of ethyl acetate. The formed white precipitate was collected by filtration and dried in vacuum at room temperature. The mentioned solid material was stirred in 80 ml of glacial acetic acid at 65° C. for 2 hours. The mixture was concentrated in vacuum to dryness and the residue was stirred in a mixture of 20 ml of methanol and 40 ml of 4 M solution of hydrogen chloride in dioxane at room temperature for 2 hours. The desired product was obtained by filtration as a white solid (9.5 g, 43%).

Individual enantiomers of this product—(1R,3S)-3-(1H-benzo[d]imidazol-2-yl)cyclohexanamine and (1S,3R)-3-(1H-benzo[d]imidazol-2-yl)cyclohexanamine—can be obtained by chromatography on a chiral column.

N-(3-(1H-benzo[d]midazol-2-yl)cyclohexyl)-2,3-dihydrobenzo[b][1,4]dioxine-6-carboxamide. To a stirred mixture of 3-(1H-benzo[d]imidazol-2-yl)cyclohexanamine dihydrochloride (5.62 g, 19.5 mmol), triethylamine (11 ml, 78 mmol), THF (160 ml), and water (30 ml) a solution of 2,3-dihydrobenzo-[b][1,4]dioxine-6-carbonyl chloride in 40 ml of THF was added at 0° C. in 10 minutes. The resulting mixture was stirred at 0° C. for 1 hour and at room temperature for 18 hours, concentrated to one third of the initial volume, and poured into 330 ml of 2% aqueous sodium bicarbonate. The precipitate was collected by filtration and subjected to chromatography on silica gel column, eluting with 2% methanol solution in ethyl acetate, to obtain 6.13 g (83%) of the title compound as a white solid. HPLC Rt=1.48; MS: [M+H]=378. SMO % inhibition at 2 uM=107.

### Example 2

### N-((1R,3S)-3-(1H-benzo[d]imidazol-2-yl)cyclohexyl]-2,3-dihydrobenzo[b][1,4]dioxine-6-carboxamide and N-((1S,3R)-3-(1H-benzo[d]imidazol-2-yl)cyclohexyl)-2,3-dihydrobenzo[b][1,4]dioxine-6-carboxamide

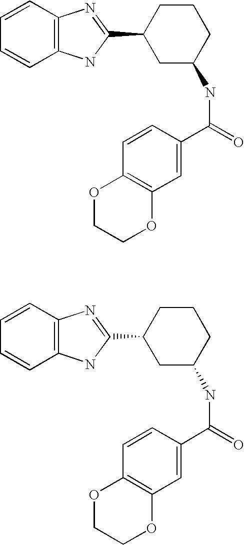

The title compounds were obtained by chiral chromatography of N-(3-(1H-benzo[d]imidazol-2-yl)cyclohexyl)-2,3-dihydrobenzo[b][1,4]dioxine-6-carboxamide (Example 1) on a modified silica gel column, eluting with 10%solution of ethanol in heptane. HPLC Rt=2.6; MS: [M+H]=408.1. SMO % inhibition at 2 uM=84.

### Example 3

### N-((1R,3S)-3-(1-methyl-1H-benzo[d]imidazol-2-yl)cyclohexyl)-2,3-dihydrobenzo[b][1,4]dioxine-6-carboxamide

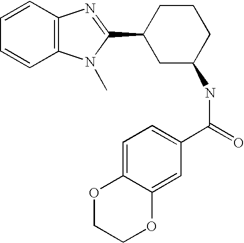

A mixture of N-((1R,3S)-3-(1H-benzo[d]imidazol-2-yl)cyclohexyl)-2,3-dihydrobenzo[b][1,4]dioxine-6-carboxamide (Example 2, 140 mg, 0.37 mmol), iodomethane (0.025 ml, 0.41 mmol), potassium carbonate (153 mg, 1.11 mmol), and DMF (3 ml) was stirred at room temperature for three hours. The reaction mixture was partitioned between 30 ml of water and 30 ml of ethyl acetate. The organic extract was dried over anhydrous magnesium sulfate and concentrated in vacuum to provide 120 mg (83%) of the title compound. 1H NMR (CD3OD) 1.5 (m,1H), 1.65 (m, 2H), 1.75 (m, 2H), 2.0 (m, 2H), 2.2 (m, 1H), 3.25 (m, 1H), 3.8 (s, 3H), 4.1 (t, 1H), 4.25 (m, 4H), 6.85 (d, 1H), 7.2 (m, 2H), 7.35 (m, 2H), 7.45 (d, 1H), and 7.55 (d, 1H). HPLC Rt=1.55. [M+H]=392. SMO % inhibition at 2 uM=107.

### Example 4

### 3,5-Dimethoxy-N-((1R,3S)-3-[5-(trifluoromethyl)-1H-benzo[d]imidazol-2-yl)cyclohexyl)benzamide

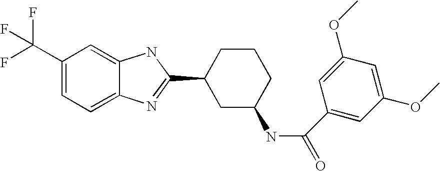

(1S,3R)-3-(Benzyloxycarbonyl)cyclohexanecarboxylic acid. To a stirred mixture of 3-amino-cyclohexanecarboxylic acid (33.0 g, 230 mmol), sodium carbonate (71.6 g, 675 mmol), and water (700 ml) a solution of benzyl chloroformate (38 ml, 266 mmol) in dioxane (175 ml) was added at 0˜5° C. during 40 min. The reaction mixture was stirred at room temperature for 18 hours, after which it was washed with 200 ml of MTBE, acidified with concentrated hydrochloric acid to pH=2, and extracted with 400 ml of ethyl acetate. The organic extract was washed with brine, dried over anhydrous magnesium sulfate, and concentrated in vacuum to obtain 60 g of white solid. The title compound (25.2 g) was isolated as a single enantiomer by chromatography of the above mentioned solid material on a modified silica gel column Chiralcel OJ-H (3 cm×25 cm), with multiple injections, eluting with a mixture of super-critical carbon dioxide and methanol (75:25). Retention time of the desired enantiomer is 4.33 minutes on the same column in the same conditions,

Benzyl(1R,3S)-3-(5-(trifluoromethyl)-1H-benzo[d]imidazol-2-yl)cyclohexylcarbamate. To a stirred mixture of (1 S,3R)-3-(benzyloxycarbonyl)cyclohexanecarboxylic acid (100 mg, 0.36 mmol), triethylamine (0.24 ml, 1.72 mmol), and DCM (4 ml) a 1M solution of iso-propyl chloroformate in toluene (0.43 ml, 0.43 mmol) was added dropwise at 0° C. The obtained solution was stirred at the same temperature for 30 minutes and was added dropwise at the same temperature to a stirred mixture of 4-(trifluoromethyl)benzene-1,2-diamine in DCM (4 ml). The resulting mixture was stirred at room temperature for 4 hours and concentrated to dryness. The residue was heated in 5 ml of glacial acetic acid at 100° C. for 2 hours. The reaction was concentrated in vacuum and partitioned between saturated sodium bicarbonate and ethyl acetate. The organic extract was concentrated and subjected to chromatography on a silica gel column, eluting with a gradient from 50 to 90% ethyl acetate in heptane to obtain 110 mg of the target product.

(1R,3S)-3-(5-(Trifluoromethyl)-1H-benzo[d]imidazol-2-yl)cyclohexanamine. A mixture of benzyl (1R,3S)-3-(5-(trifluoromethyl)-1H-benzo[d]imidazol-2-yl)cyclohexylcarbamate (110 mg, 0.26 mmol), 10% palladium on activated carbon, and methanol (30 ml) was shaken under 40 psi of hydrogen gas at room temperature for 18 hours. The mixture was filtered through a pad of Celite and concentrated in vacuum to provide the desired product as a yellow glass, 70% pure by analytical HPLC.

3,5-Dimethoxy-N-((1R,3S)-3-[5-(trifluoromethyl)-1H-benzo[d]imidazol-2-yl)cyclohexyl)benzamide. To a stirred mixture of 3,5-dimethoxybenzoic acid (46 mg, 0.25 mmol), triethylamine (0.116 ml, 0.84 mmol), and DCM (5 ml) a IM solution of iso-propyl chloroformate in toluene (0.25 ml, 0.25 mmol) was added dropwise at 0° C. The obtained solution was stirred at the same temperature for 30 minutes and was added dropwise at the same temperature to a stirred mixture of (1R,3S)-3-(5-(trifluoromethyl)-1H-benzo[d]imidazol-2-yl)cyclohexanamine (70% pure by analytical HPLC, 60 mg, 0.21 mmol) in DMF (2 ml). The resulting mixture was stirred at room temperature for 4 hours and after this partitioned between DCM and saturated sodium bicarbonate. The organic phase was concentrated and chromatographed on silica gel column, eluting with a gradient from 60 to 100% ethyl acetate in heptane to obtain 35 mg (53%) of the title compound. HPLC Rt=2.9. MS: (M+H]=448.4. SMO % inhibition at 2 uM=104.94.

### Example 5

### 3,5-dimethoxy-N-[3-(5-methyl-1H-benzo[d]imidazol-2-yl)cyclohexyl)benzamide

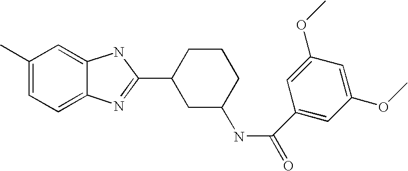

3-(3,5-Dimethoxybenzamido)cyclohexanecarboxylic acid. A mixture of 3-(3,5-dimethoxybenzamido)-cyclohexanecarboxylic acid (1.47 g, 8.07 mmol), thionyl chloride (0.71 ml, 9.7 mmol), DMF (0.03 ml, 0.38 mmol), and DCM (15 ml) was stirred at 42° C. for 2 hours, after which it was concentrated in vacuum and dried in high vacuum. A solution of the obtained solid in 10 ml of THF was added to a stirred mixture of 3-aminocyclohexanecarboxylic acid (1.26 g, 8.8 mmol), thriethylamine (3.3 ml, 24 mmol), THF (15 ml), and water (15 ml) at 0° C. in 5 min. The stirring continued for 1 hour at the same temperature and for 3 hours at room temperature. After this, 10 ml of 2 M aqueous hydrochloric acid was added. The mixture was extracted with 30 ml of ethyl acetate. A precipitate appeared on standing. The extract was concentrated to dryness and dried in high vacuum to obtain 2.3 g (93%) of the target product as a white solid.

3,5-Dimethoxy-N-(3-(5-methyl-1H-benzo[d]imidazol-2-yl)cyclohexyl)benzamide. A mixture of 3-(3,5-dimethoxybenzamido)cyclohexanecarboxylic acid (42 mg, 0.137 mmol), 4-methylbenzene-1,2-diamine (20 mg, 0.16 mmol), benzotriazol-1-yloxy)tris(dimethylamino)phosphonium hexafluorophosphate (73 mg, 0.16 mmol), diisopropylethylamine (0.049 ml, 0.28 mmol), and DMF (1 ml) was stirred at room temperature for 18 hours. The reaction mixture was poured into 10 ml of water, the precipitate was separated and subjected to heating in 1 ml of glacial acetic acid at 105° C. for 1 hour. Chromatography on reverse phase column, eluting with agradient fro 10 to 60% acetonitrile in 0.1% aqueous formic acid provided 13.8 mg of the title compound. HPLC Rt=2.6. MS: [M+H]=394.3. SMO % inhibition at 2 uM=102.

### Example 6

### N-(3-(1-Ethyl-1H-benzo[d]imidazol-2-yl)cyclohexyl)-2,3-dihydrobenzo[b][1,4]dioxine-6-carboxamide

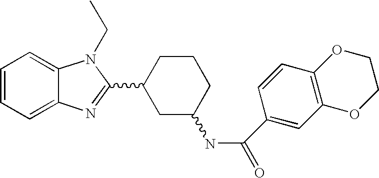

A mixture of N-(3-(1H-benzo[d]imidazol-2-yl)cyclohexyl)-2,3-dihydrobenzo[b][1,4]dioxine-6-carboxamide (Example 1, 30 mg, 0.08 mmol), iodoethane (0.032 ml, 0.4 mmol), potassium carbonate (33 mg, 0.24 mmol), and DMF (1 ml) was stirred at 65° C. for 18 hours. Solids were filtered off. Chromatography on reverse phase column, eluting with a gradient from 10 to 60% acetonitrile in 0.1% aqueous formic acid provided 17.5 mg of the title compound. HPLC Rt=2.2. MS: [M+H]=406.1. SMO % inhibition at 2 uM=58.

### Example 7

### 2-(3-[3,5-dimethoxybenzamido)cyclohexyl)-N-methyl-1H-benzo[d]imidazole-5-carboxamide

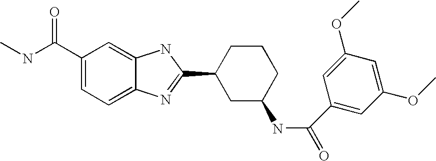

A mixture of 2-(3-(3,5-dimethoxybenzamido)cyclohexyl)-1H-benzo[d]imidazole-5-carboxylic acid (obtained similarly to Example 5 starting from ethyl 3,4-diaminobenzoate) −25 mg, 0.06 mmol, 2 M solution of methylamine in THF (0.15 ml, 0.3 mmol), ), benzotriazol-1-yloxy)tris(dimethylamino)-phosphonium hexafluorophosphate (53 mg, 0.12 mmol), and DMF (1 ml) was stirred at room temperature for 2 days. Chromatography on reverse phase column, eluting with a gradient from 5 to 60% acetonitrile in 0.1% aqueous formic acid gave 11 mg of the cis-title compound. HPLC Rt=2.1. MS: [M+H]=437. SMO % inhibition at 2 uM=70.

### Example 8

### 2-(3-(3,5-dimethoxybenzamido)cyclohexyl)-N,N-dimethyl-1H-benzo[d]imidazole-5-carboxamides (cis- and trans-isomers)

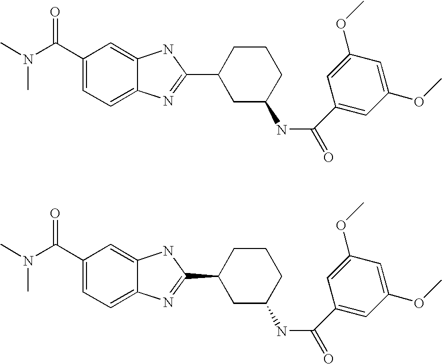

The procedure similar to the one described in Example 7 led to a crude product which after chromatography on a reverse phase column gave cis- and trans-enantiomeric pairs of the title compound (10 mg and 3 mg, respectively). HPLC Rt=2.5. MS: [M+H]=451.4. SMO % inhibition at 2 uM=68.

### Example 9

### N-(3-(5-Acetamido-1H-benzo[d]imidazol-2-yl)cyclohexyl)-3,5-dimethoxybenzamide

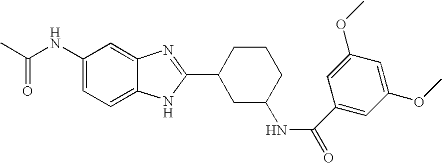

N-(3-(5-Amino-1H-benzo[d]imidazol-2-yl)cyclohexyl)-3,5-dimethoxybenzamide. A mixture of 3,5-dimethoxy-N-(3-(5-nitro-1H-benzo[d]imidazol-2-yl)cyclohexyl)benzamide (prepared in a manner similar to Example 5) -200 mg, 0.47 mmol, 10% palladium on activated carbon (200 mg), and methanol (30 ml) was shaken under 40 psi of hydrogen gas at room temperature for 1 hour. The mixture was filtered through a pad of Celite and concentrated in vacuum. Chromatography on silica gel, eluting with a mixture of ethyl acetate—methanol—38% aqueous ammonia (95:5:0.5) gave 160 mg of the desired product.

N-(3-(5-Acetamido-1H-benzo[d]imidazol-2-yl)cyclohexyl)-3,5-dimethoxybenzamide. To a stirred mixture of N-(3-(5-amino-1H-benzo[d]imidazol-2-yl)cyclohexyl)-3,5-dimethoxybenzamide (30 mg, 0.076 mmol), pyridine (0.2 ml), and DCM (1 ml) neat acetyl chloride (0.012 ml, 0.152 ml) was added in one portion at room temperature. After 15 minutes the reaction mixture was concentrated and the residue was chromatographed on reverse phase column, eluting with a gradient fro 10 to 60% of acetonitrile in 0.1% aqueous formic acid to obtain 10 mg of the title compound. HPLC Rt=224. MS: [M+H]=437.0. SMO % inhibition at 2 uM=87.

### Example 10

### N-(3-(5-Isobutyramido-1H-benzo[d]imidazol-2-yl)cyclohexyl)-3,5-dimethoxybenzamide

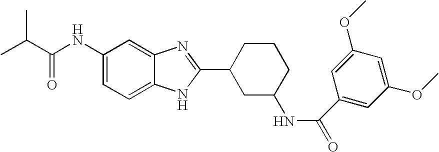

The title compound was obtained in 7.6 mg yield starting from 30 mg of N-(3-(5-amino-1H-benzo[d]imidazol-2-yl)cyclohexyl)-3,5-dimethoxybenzamide according to the procedure in Example 9. HPLC Rt=2.6. MS: [M+H]=465.1. SMO % inhibition at 2 uM=96.

### Example 11

### 3,5-Dimethoxy-N-(3-(5-(methylsulfonamido)-1H-benzo[d]imidazol-2-yl)cyclohexyl)benzamide

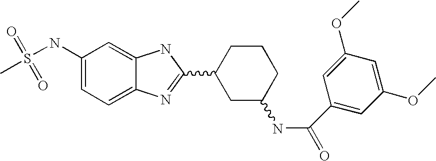

To a stirred mixture of N-(3-(5-amino-1H-benzo[d]imidazol-2-yl)cyclohexyl)-3,5-dimethoxybenzamide (30 mg, 0.076 mmol), pyridine (0.2 ml), and DCM (1 ml) neat methanesulfonyl chloride (0.012 ml, 0.152 ml) was added in one portion at room temperature. After 15 min the reaction mixture was concentrated and the residue was heated in a stirred mixture of 2 ml of THF and 1 ml of 1 M aqueous lithium hydroxide at 60° C. for 6 hours. The title compound (1.9 mg) was obtained by chromatography on reverse phase column, eluting with a gradient from 5 to 60% of acetonitrile in 0.1% aqueous formic acid. HPLC Rt=2.4. MS: [M+H]=473. SMO % inhibition at 2 uM=86.

### Example 12

### 3,5-Dimethoxy-N-(3-(5-(propan-2-ylsulfonamido)-1H-benzo[d]imidazol-2-yl)cyclohexyl)

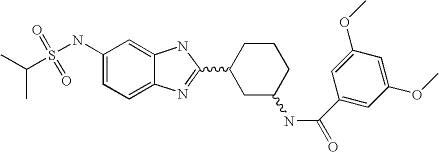

The title compound was obtained in 7.9 mg yield starting from 30 mg of N-(3-(5-amino-1H-benzo[d]imidazol-2-yl)cyclohexyl)-3,5-dimethoxybenzamide according to the procedure in Example 11.HPLC Rt=2.6. MS: [M+H]=501. SMO % inhibition at 2 uM=62.

### Example 13

### N-(3-(1-(2-Hydroxyethyl)-1H-benzo[d]imidazol-2-yl)cyclohexyl)-3,5-dimethoxybenzamide (cis-isomer)

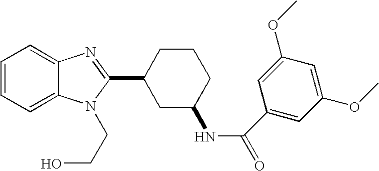

N-(3-(1-(2-(tert-Butyldimethylsilyloxy)ethyl)-1H-benzo[d]imidazol-2-yl)cyclohexyl)-3,5-dimethoxy-benzamide. A mixture of N-(3-(1H-benzo[d]imidazol-2-yl)cyclohexyl)-2,3-dihydrobenzo[b][1,4]dioxine-6-carboxamide (Example 1, 140 mg, 0.37 mmol), (2-bromoethoxy)(tert-butyl)dimethylsilane (0.16 ml, 0.74 mmol), potassium carbonate (102 mg, 0.74 mmol), and DMF (3 ml) was stirred at 95° C. for 3 days. The mixture was partitioned between 15 ml of water and 20 ml of ethyl acetate. The organic extract was concentrated and chromatographed on silica gel, eluting with a gradient from 60 to 100% ethyl acetate in heptane to obtain 163 mg of the desired product.

N-(3-(1-(2-Hydroxyethyl)-1H-benzo[d]imidazol-2-yl)cyclohexyl)-3,5-dimethoxybenzamide (cis-isomer). A mixture of N-(3-(1-(2-(tert-butyidimethylsilyloxy)ethyl)-1H-benzo[d]imidazol-2-yl)cyclohexyl)-3,5-dimethoxy-benzamide, 1 M solution of tetrabuthylammonium fluoride in THF (1.2 ml, 1.2 ml, mol), and THF (2 ml) was stirred at room temperature for 2 h, concentrated to about 1/5 of the initial volume. Successive chromatography on silica gel (form 0 to 5% methanol in ethyl acetate) and on a reverse phase column (from 5 to 60% acetonitrile in 0.1% aqueous formic acid) provided 80 mg of the title compound as a mixture of cis-enantiomers. HPLC Rt=2.0. MS: [M+H]=422.3. SMO % inhibition at 2 uM=98.

### Example 14

### N-Methyl-N-(3-(1-methyl-1H-benzo[d]imidazol-2-yl)cyclohexyl)-2,3-dihydrobenzo[b][1,4]dioxine-6-carboxamide

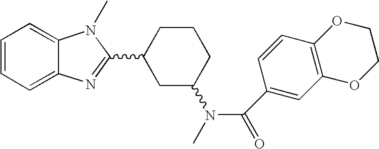

To a stirred mixture of N-(3-(1H-benzo[d]imidazol-2-yl)cyclohexyl)-2,3-dihydrobenzo[b][1,4]dioxine-6-carboxamide (Example 1,22 mg, 0.058 mmol) and DMF (1.5 ml) a 1 M solution of potassium tert-butoxide in THF (0.23 ml, 0.23 mmol) was added at room temperature in one portion and the obtained mixture was stirred at room temperature for 1 hour. Iodomethane (0.015 ml, 0.23 mmol) was added in one portion and stirring continued at room temperature for 24 hours. Solids were filtered off and 8 ml of water was added. The mixture was extracted with 2 ml of ethyl acetate. Chromatography on silica gel, eluting with a gradient from 70 to 100% ethyl acetate in heptane provided 7 mg of the title compound. HPLC Rt 1.9. MS: [M+H]=406. SMO % inhibition at 2 uM=95.

### Example 15

### N-(3-(1-(2-Cyanoethyl)-1H-benzo[d]imidazol-2-yl)cyclohexyl)-2,3-dihydrobenzo[b][1,4]dioxine-6-carboxamide

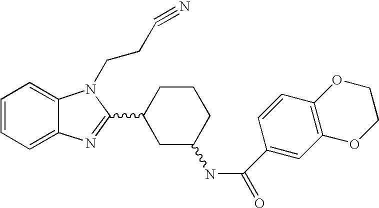

A mixture of N-(3-(1H-benzo[d]imidazol-2-yl)cyclohexyl)-2,3-dihydrobenzo[b][1,4]dioxine-6-carboxamide (Example 1, 30 mg, 0.08 mmol), 3-bromopropionitrile (0.013 ml, 0.16 mmol), potassium carbonate (33 mg, 0.24 mmol), and DMF (1 ml) was stirred at 100° C. for 18 hours. Solids were filtered off. Chromatography on reverse phase column, eluting with a gradient from 5 to 60% acetonitrile in 0.1% aqueous formic acid provided 2.4 mg of the title compound. HPLC Rt=2.3. MS: [M+H]=421.3. SMO % inhibition at 2 uM=94.

### Example 16

### N-(3-(1-(2-Methoxyethyl)-1H-benzo[d]imidazol-2-yl]cyclohexyl)-2,3-dihydrobenzo[b][1,4]dioxine-6-carboxamide

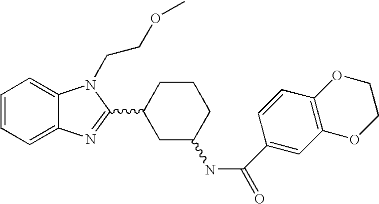

A mixture of N-(3-(1H-benzo[d]imidazol-2-yl)cyclohexyl)-2,3-dihydrobenzo[b][1,4]dioxine-6-carboxamide (Example 1, 30 mg, 0.08 mmol), 1-bromo-2-methoxyethane (0.013 ml, 0.16 mmol), potassium carbonate (33 mg, 0.24 mmol), and DMF (1 ml) was stirred at 100° C. for 18 hours. Solids were filtered off. Chromatography on reverse phase column, eluting with a gradient from 5 to 60% acetonitrile in 0.1% aqueous formic acid provided 2.3 mg of the title compound. HPLC Rt=2.4. MS: [M+H]=436.3. SMO % inhibition at 2 uM=59.

### Example 17

### N-(3-(5-Bromo-1H-benzo[d]imidazol-2-yl)cyclohexyl)-3,5-dimethoxybenzamide

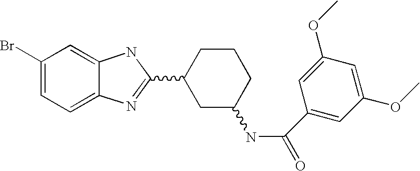

The title compound was prepared according to the procedure described in Example 5. HPLC Rt=2.7. MS: [M+H]=457.9. SMO % inhibition at 2 uM=103.

### Example 18

### N-(3-(5-Bromo-1-methyl-1H-benzo[d]imidazol-2-yl)cyclohexyl)-3,5-dimethoxybenzamide and N-(3-(6-bromo-1-methyl-1H-benzo[d]imidazol-2-yl)cyclohexyl)-3,5-dimethoxybenzamide

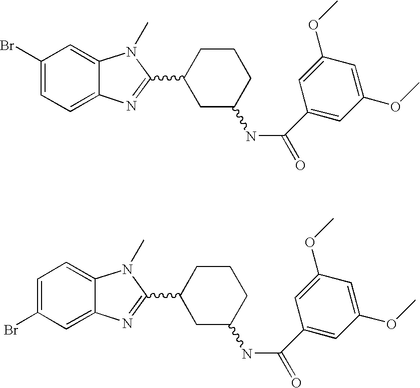

A mixture (1:1) of the title compounds was prepared according to Example 3 from N-(3-(5-bromo-1H-benzo[d]imidazol-2-yl)cyclohexyl)-3,5-dimethoxybenzamide (Example 17). HPLC Rt=3. MS: [M+H]=472.3. SMO % inhibition at 2 uM=107.

### Example 19

### N-(3-(5-Cyano-1H-benzo[d]imidazol-2-yl)cyclohexyl]-2,3-dihydrobenzo[b][1,4]dioxine-6-carboxamide

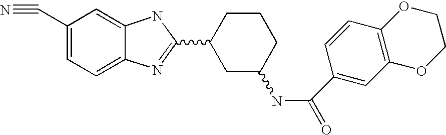

The title compound was prepared according to the procedure described in Example 5. HPLC Rt=2.7. MS: [M+H]=403. SMO % inhibition at 2 uM=100.

### Example 20

### N-(3-(5-Cyano-1-methyl-1H-benzo[d]imidazol-2-yl)cyclohexyl]-2,3-dihydrobenzo[b][1,4]dioxine-6-carboxamide and N-(3-(6-Cyano-1-methyl-1H-benzo[d]imidazol-2-yl)cyclohexyl]-2,3-dihydrobenzo[b][1,4]dioxine-6-carboxamide

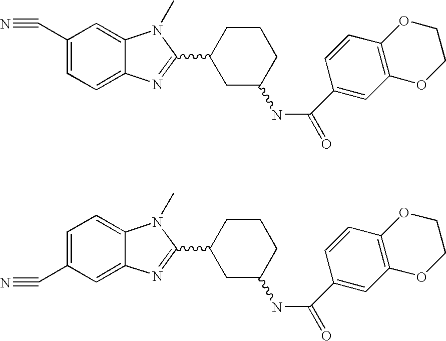

A mixture (1:1) of the title compounds was prepared according to Example 3 from N-(3-(5-cyano-1H-benzo[d]imidazol-2-yl)cyclohexyl)-2,3-dihydrobenzo[b][1,4]dioxine-6-carboxamide (Example 19). HPLC Rt=2.8. MS: [M+H]=417. SMO % inhibition at 2 uM=103.

### Example 21

### N-((1S,3R)-3-(1-Methyl-1H-benzo[d]imidazol-2-yl)cyclohexyl)-2,3-dihydrobenzo[b](1,4]dioxine-6-carboxamide

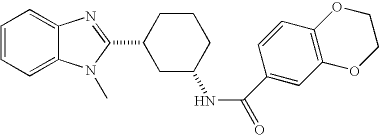

The title compound was prepared according to Example 3 from N-((1S,3R)-3-(1H-benzo[d]imidazol-2-yl)cyclohexyl)-2,3-dihydrobenzo[b](1,4]dioxine-6-carboxamide (Example 2). HPLC Rt=1.7. MS: [M+H]=392.0. SMO % inhibition at 2 uM=71.

### Example 22

### N-(3-(1,5-Dimethyl-1H-benzo[d]imidazol-2-yl)cyclohexyl]-3,5-dimethoxybenzamide and N-(3-(1,6-dimethyl-1H-benzo[d]imidazol-2-yl)cyclohexyl]-3,5-dimethoxybenzamide

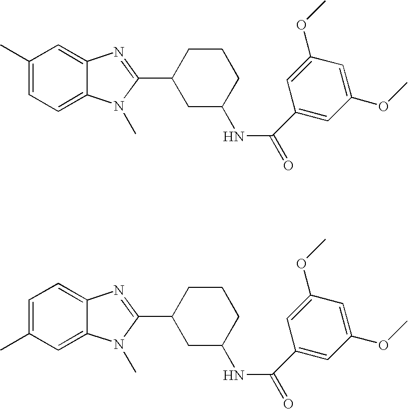

A mixture (1:1) of the title compounds was prepared according to Example 3 from 3,5-dimethoxy-N-(3-(5-methyl-1H-benzo[d]imidazol-2-yl)cyclohexyl)benzamide (Example 5). HPLC Rt=2.6. MS: [M+H]=408.1. SMO % inhibition at 2 uM=98.

### Example 23

### N-(3-(5-(Aminomethyl)-1-methyl-1H-benzo[d]imidazol-2-yl)cyclohexyl)-3,5-dimethoxybenzamide and N-(3-(6-(aminomethyl)-1-methyl-1H-benzo[d]imidazol-2-yl)cyclohexyl)-3,5-dimethoxybenzamide

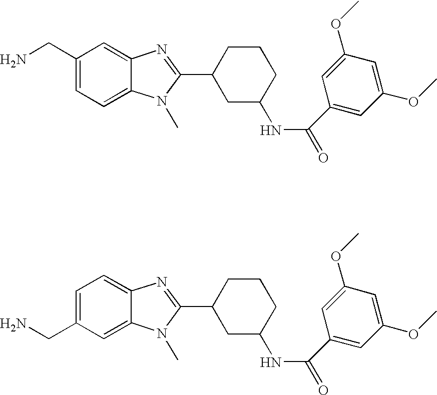

To a stirred solution of a mixture (1:1) of N-(3-(5-cyano-1-methyl-1H-benzo[d]imidazol-2-yl)cyclohexyl )-2,3-dihydrobenzo[b][1,4]dioxine-6-carboxamide and N-(3-(6-cyano-1-methyl-1H-benzo[d]imidazol-2-yl)cyclohexyl)-2,3-dihydrobenzo[b][1,4]dioxine-6-carboxamide (Example 20, 140 mg, 0.34 mmol)) and THF (3 ml) a 1 M solution of lithium aluminum hydride (0.67 ml, 0.67 mmol) was added dropwise at room temperature and the obtained reaction mixture was stirred at room temperature for 24 hours. Water (0.3 ml), ethyl acetate (10 ml), and silica gel (1 g) were added and the resulting mixture was stirred at room temperature for 1 hour. Solids were filtered off. Chromatography on a silica gel column, eluting with a gradient from 2 to 10% of a mixture methanol-aqueous ammonia (10:1) in ethyl acetate gave 30 mg of 1:1 mixture of the title products. HPLC Rt=2.6. MS: [M+H]=420.1. SMO % inhibition at 2 uM=104.

### Example 24

### 3,5-Dimethoxy-N-(3-(6-(trifluoromethyl)-1H-benzo[d]imidazol-2-yl]cyclohexyl)benzamide

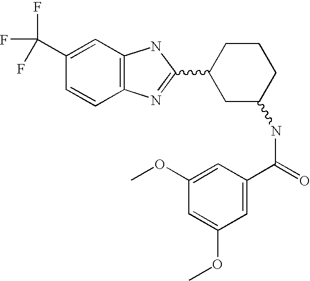

The title compound was prepared similarly to the procedure described in Example 5. HPLC Rt=2.8; MS: [M+H]=448.1. SMO % inhibition at 2 uM=105.

### Example 25

### N-(3-(6-Chloro-1H-benzo[d]imidazol-2-yl)cyclohexyl]-3,5-dimethoxybenzamide

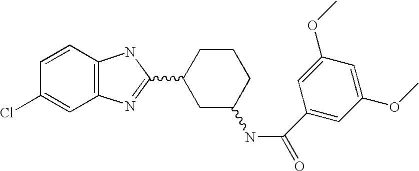

The title compound was prepared similarly to the procedure described in Example 5. HPLC Rt=2.7; MS: [M+H]=414. SMO % inhibition at 2 uM=99.

### Example 26

### N-(3-(1,6-Dimethyl-1H-benzo[d]imidazol-2-yl)cyclohexyl]-3,5-dimethoxybenzamide

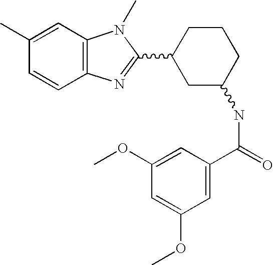

N,5-Dimethyl-2-nitrobenzenamine. A mixture of 2-fluoro-4-methyl-1-nitrobenzene (2.55 g, 16.4 mmol) and 10% solution of methylamine in ethanol (20 ml) was stirred at 80° C. for 2 hours. After cooling to room temperature, 50 ml of water was added and the resulting slurry was stirred at room temperature for 18 hours. The target product was isolated by filtration as an orange solid (2.4 g, 88% yield).

N¹,5-Dimethylbenzene-1,2-diamine. A mixture of N,5-dimethyl-2-nitrobenzenamine (2.4 g, 14.4 mmol), 10% palladium on activated carbon (100 mg), and methanol (60 ml) was shaken under 40 psi of hydrogen gas during 1 hour. The mixture was filtered through a pad of Celite and concentrated in vacuum to provide 1.9 g of the desired product.

N-(3-(1,6-Dimethyl-1H-benzo[d]imidazol-2-yl)cyclohexyl]-3,5-dimethoxybenzamide. The title product was prepared similarly to the procedure described in Example 5 from 3-(3,5-dimethoxybenzamido)-cyclohexanecarboxylic acid and N¹,5-dimethylbenzene-1,2-diamine. HPLC Rt=2.6; MS: [M+H]=408.1. SMO % inhibition at 2 uM=113.

### Example 27

### N-(3-(1,5-Dimethyl-1H-benzo[d]imidazol-2-yl)cyclohexyl]-3,5-dimethoxybenzamide

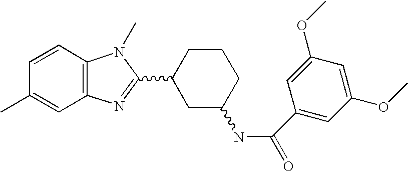

The title compound was prepared similarly to the procedure described in Example 26, starting from 1-fluoro-4-methyl-2-nitrobenzene. HPLC Rt=2.6; MS: [M+H]=408.1. SMO % inhibition at 2 uM=84.

### Example 28

### N-(3-(5-Methyl-1H-benzo[d]imidazol-2-yl)cyclohexyl]-2,3-dihydrobenzo[b][1,4]dioxine-6-carboxamide

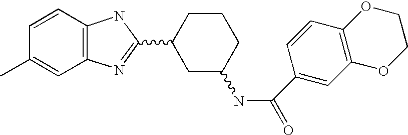

3-(2,3-Dihydrobenzo[b][1,4]dioxine-7-carboxamido)cyclohexanecarboxylic acid. This intermediate was prepared similarly to 3-(3,5-dimethoxybenzamido)cyclohexanecarboxylic acid (see Example 5).

N-(3-(5-Methyl-I H-benzo[d]imidazol-2-yl)cyclohexyl]-2,3-dihydrobenzo[b][1,4]dioxine-6-carboxamide. To a stirred mixture of 3-(2,3-dihydrobenzo[b][1,4]dioxine-7-carboxamido)-cyclohexanecarboxylic acid (50 mg, 0.16 mmol), triethylamine (0.09 ml, 0.64 mmol), and DCM (1 ml) iso-butyl chloroformate (0.04 ml, 0.32 mmol) was added dropwise at room temperature. After 10 minutes a solution of 4-methylbenzene-1,2-diamine (39 mg, 0.32 mmol) in DCM (0.5 ml) was added and the resulting mixture was stirred at room temperature for 30 minutes. The reaction mixture was concentrated to dryness and the residue was heated in glacial acetic acid (1 ml) at 100° C. during 30 minutes. The solution was concentrated to dryness and the residue was partitioned between saturated aqueous sodium carbonate and ethyl acetate. The organic extract was concentrated, and chromatography on a silica gel column, eluting a gradient from 80 to 100% of ethyl acetate in heptane gave 40 mg of the title compound. HPLC Rt=2.4; MS: [M+H]=392.1. SMO % inhibition at 2 uM=103.

### Example 29

### N-(3-(5-Methoxy-1H-benzo[d]imidazol-2-yl)cyclohexyl]-2,3-dihydrobenzo[b][1,4]dioxine-6-carboxamide

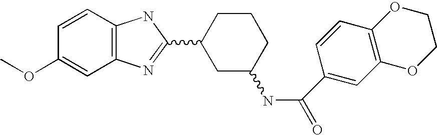

The title compound was prepared in a manner similar to the procedure described in Example 28. HPLC Rt=2.4; MS: [M+H]=408.1. SMO % inhibition at 2 uM=102.

### Example 30

### N-(3-(6-(Hydroxymethyl)-1-methyl-1H-benzo[d]imidazol-2-yl)cyclohexyl)-2,3-dihydrobenzo[b][1,4]dioxine-6-carboxamide and N-(3-(5-(Hydroxymethyl)-1-methyl-1H-benzo[d]imidazol-2-yl)cyclohexyl)-2,3-dihydrobenzo[b][1,4]dioxine-6-carboxamide

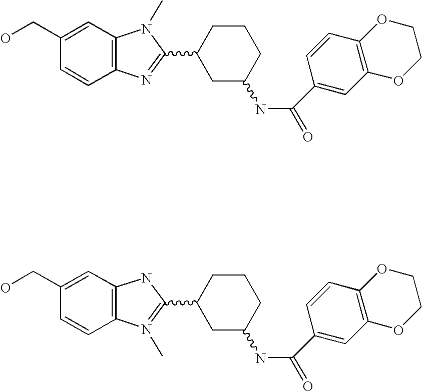

Ethyl 2-(3-(2,3-dihydrobenzo[b][1,4]dioxine-7-carboxamido)cyclohexyl)-1H-benzo[d]imidazole-5-carboxylate. This intermediate was prepared in a manner similar to the procedure described in Example 5.

Ethyl 2-(3-(2,3-dihydrobenzo[b][1,4]dioxine-7-carboxamido)cyclohexyl)-3-methyl-3H-benzo[d]-imidazole-5-carboxylate and ethyl 2-(3-(2,3-dihydrobenzo[b][1,4]dioxine-7-carboxamido)cyclohexyl)-1-methyl-1H-benzo[d]imidazole-5-carboxylate. These intermediates were prepared as a 1:1 mixture in a fashion similar to the procedure described in Example 3 from ethyl 2-(3-(2,3-dihydrobenzo[b][1,4]dioxine-7-carboxamido)cyclohexyl)-1H-benzo[d]imidazole-5-carboxylate.

N-(3-(6-(Hydroxymethyl)-1-methyl-1H-benzo[d]imidazol-2-yl)cyclohexyl)-2,3-dihydrobenzo[b)[1,4]dioxine-6-carboxamide and N-(3-(5-(Hydroxymethyl)-1-methyl-1H-benzo[d]imidazol-2-yl)cyclohexyl)-2,3-dihydrobenzo[b][1,4]dioxine-6-carboxamide. To a stirred 1:1 mixture of ethyl 2-(3-(2,3-dihydrobenzo[b][1,4]dioxine-7-carboxamido)cyclohexyl)-3-methyl-3H-benzo[d]-imidazole-5-carboxylate and ethyl 2-(3-(2,3-dihydrobenzo[b][1,4]dioxine-7-carboxamido)cyclohexyl)-1-methyl-1H-benzo[d]imidazole-5-carboxylate (600 mg, 1.29 mmol) in THF (6 ml) a solution of lithium aluminum hydride in THF (1.6 ml, 1.6 mmol) was added at 0° C. during 15 minutes. The reaction mixture was stirred at the same temperature during 1 hour and at room temperature during 18 hours. Water (0.5 ml) and ethyl acetate (10 ml) were added successively and stirring continued for 30 minutes. The mixture was loaded on silica gel. Chromatography on silica gel column, eluting with a gradient from 1 to 10% methanol in ethyl acetate yielded 240 mg of the title compound. 1H NMR (d6-acetone) (methanol-d4, 400 MHz) 1.4-1.5 (m, 1H), 1.55-1.65 (m, 1H), 1.65-1.75 (m, 2 H), 1.95-2.1 (m, 4H), 3.15-3.25 (m, 1H), 3.8 (s, 3H), 4.0-4.1 (m, 1H), 4.2, (s, 4H), 4.7 (s, 2H), 6.85 (d, 1H), 7.25 (d, 1H), 7.3-7.35 (m, 2H), 7.4-7.45 (m, 1H), 7.5 (d, 1H). HPLC Rt=1.9; MS: [M+H]=422.1. SMO % inhibition at 2 uM=104.

### Example 31

### N-(3-(6-(2-Hydroxypropan-2-yl)-1-methyl-1H-benzo[d]imidazol-2-yl)cyclohexyl)-2,3-dihydrobenzo[b][1,4]dioxine-6-carboxamide and N-(3-(5-(2-Hydroxypropan-2-yl)-1-methyl-1H-benzo[d]imidazol-2-yl)cyclohexyl)-2,3-dihydrobenzo[b][1,4]dioxine-6-carboxamide

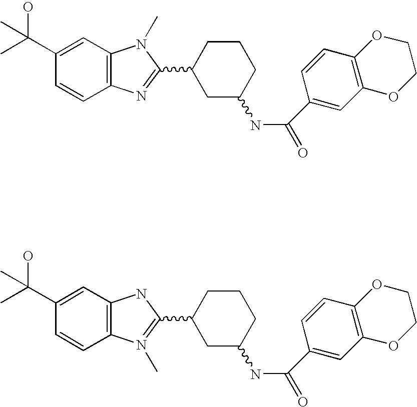

To a stirred 1:1 mixture of ethyl 2-(3-(2,3-dihydrobenzo[b][1,4]dioxine-7-carboxamido)cyclohexyl)-3-methyl-3H-benzo[d]-imidazole-5-carboxylate and ethyl 2-(3-(2,3-dihydrobenzo[b][1,4]dioxine-7-carboxamido)cyclohexyl)-1-methyl-1H-benzo[d]imidazole-5-carboxylate (65 mg, 0.14 mmol) in THF (3 ml) a 3.0 M solution of methylmagnesium bromide in THF (0.19 ml, 0.56 mmol) was added at 0° C. during 5 minutes. The reaction mixture was stirred at the same temperature during 1 hour, warmed to 15° C. and quenched with 0.3 ml of water at this temperature. The obtained mixture was loaded on silica gel and chromatographed on a silica gel column, eluting with a gradient from 0 to 5% methanol in ethyl acetate to obtain 11 mg of 1:1 mixture of the title products. HPLC Rt=1.2; MS: [M+H]=450.1. SMO % inhibition at 2 uM=97.

### Example 32

N-(3-(6-Isopropyl-1-methyl-1H-benzo[d]imidazol-2-yl)cyclohexyl)-2,3-dihydrobenzo[b][1,4]dioxine-6-carboxamide and N-(3-(5-isopropyl-1-methyl-1H-benzo[d]imidazol-2-yl)cyclohexyl)-2,3-dihydrobenzo[b][1,4]dioxine-6-carboxamide

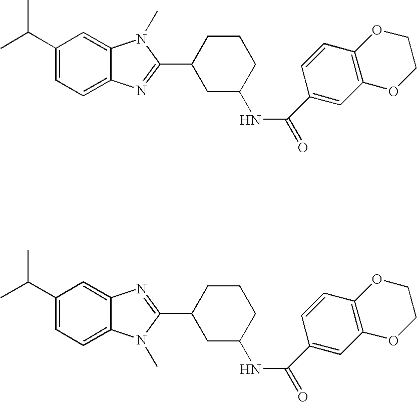

The title compounds were isolated as a 1:1 mixture as by-products of the preparation in Example 31. HPLC Rt=1.7. MS: [M+H]=434.1. SMO % inhibition at 2 uM=81.

### Example 33

### N-(3-(6-(Methoxymethyl)-1-methyl-1H-benzo[d]imidazol-2-yl]cyclohexyl)-2,3-dihydrobenzo[b][1,4]dioxine-6-carboxamide and N-(3-(6-(Methoxymethyl)-1-methyl-1H-benzo[d]imidazol-2-yl]cyclohexyl)-2,3-dihydrobenzo[b][1,4]dioxine-6-carboxamide

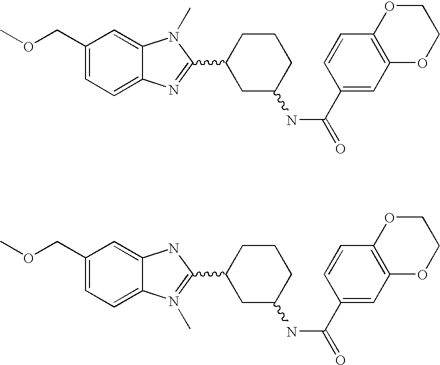

N-(3-(6-(Chloromethyl)-1-methyl-1H-benzo[d]imidazol-2-yl)cyclohexyl)-2,3-dihydrobenzo-[b)[1,4]dioxine-6-carboxamide and N-(3-(5-(chloromethyl)-1-methyl-1H-benzo[d]imidazol-2-yl)cyclohexyl)-2,3-dihydrobenzo b][1,4]dioxine-6-carboxamide. To a stirred 1:1 mixture of N-(3-(6-(hydroxymethyl)-1-methyl-1H-benzo[d]imidazol-2-yl)cyclohexyl)-2,3-dihydrobenzo[b][1,4]-dioxine-6-carboxamide and N-(3-(5-(hydroxymethyl)-1-methyl-1H-benzo[d]imidazol-2-yl)-cyclohexyl)-2,3-dihydrobenzo[b][1,4]dioxine-6-carboxamide (220 mg, 0.52 mmol), triethylamine (0.22 ml, 1.56 mmol), and DCM (10 ml) neat methanesulfonyl chloride (0.049 ml, 0.63 mmol) was added dropwise at 0° C. and the stirring continued during 2 hours at the same temperature. Water (10 ml) was added, and after vigorous stirring the organic phase was separated, dried over magnesium sulfate and concentrated to provide a 1:1 mixture of the target compounds, which was immediately used for the next step without additional purification.

N-(3-(6-(Methoxymethyl)-1-methyl-1H-benzo[d]imidazol-2-yl]cyclohexyl)-2,3-dihydrobenzo[b][1,4]dioxine-6-carboxamide and N-(3-(6-(Methoxymethyl)-1-methyl-1H-benzo[d]imidazol-2-yl]cyclohexyl)-2,3-dihydrobenzo[b][1,4]dioxine-6-carboxamide. A 1:1 mixture of N-(3-(6-(chloromethyl)-1-methyl-1H-benzo[d]imidazol-2-yl)cyclohexyl)-2,3-dihydrobenzo-[b][1,4]dioxine-6-carboxamide and N-(3-(5-(chloromethyl)-1-methyl-1H-benzo[d]imidazol-2-yl)cyclohexyl)-2,3-dihydrobenzo[b][1,4]dioxine-6-carboxamide (0.07 mmol), was stirred with DIPEA (0.024 ml, 0.14 mmol), and methanol (1.2 ml) at 50° C. for four hours. The title products were isolated as a 1:1 mixture (8 mg) by chromatography on a reverse phase column, eluting with a gradient from 5 to 60% of acetonitrile in 0.1% aqueous formic acid. HPLC Rt=1.6; MS: [M+H]=436.1. SMO % inhibition at 2 uM=102.

### Example 34

### N-(3-(6-((Dimethylamino)methyl]-1-methyl-1H-benzo[d]imidazol-2-yl)cyclohexyl)-2,3-dihydrobenzo[b][1,4]dioxine-6-carboxamide and N-(3-(5-((Dimethylamino)methyl]-1-methyl-1H-benzo[d]imidazol-2-yl)cyclohexyl)-2,3-dihydrobenzo[b][1,4]dioxine-6-carboxamide

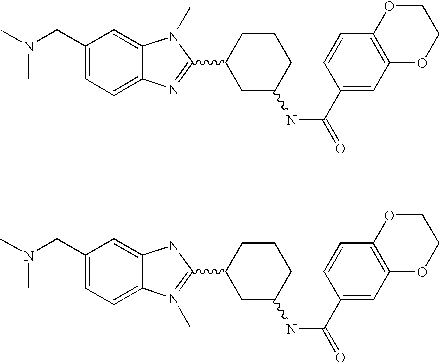

A 1:1 mixture of N-(3-(6-(chloromethyl)-1-methyl-1H-benzo[d]imidazol-2-yl)cyclohexyl)-2,3-dihydrobenzo-[b][1,4]dioxine-6-carboxamide and N-(3-(5-(chloromethyl)-1-methyl-1H-benzo[d]imidazol-2-yl)cyclohexyl)-2,3-dihydrobenzo[b][1,4]dioxine-6-carboxamide (0.07 mmol), was stirred with a 2.0 M solution of dimethylamine in THF (1.2 ml, 2.4 mmol) at room temperature for 18 hours. The title products were isolated as a 1:1 mixture (12 mg) by chromatography on a reverse phase column, eluting with a gradient from 5 to 60% of acetonitrile in 0.1% aqueous formic acid. HPLC Rt=1.1; MS: [M+H]=449.1. SMO % inhibition at 2 uM=96.

### Example 35

### N-(3-(6-(Cyanomethyl)-1-methyl-1H-benzo[d]imidazol-2-yl)cyclohexyl)-2,3-dihydrobenzo[b][1,4]dioxine-6-carboxamide and N-(3-(5-(Cyanomethyl)-1-methyl-1H-benzo[d]imidazol-2-yl)cyclohexyl)-2,3-dihydrobenzo[b][1,4]dioxine-6-carboxamide

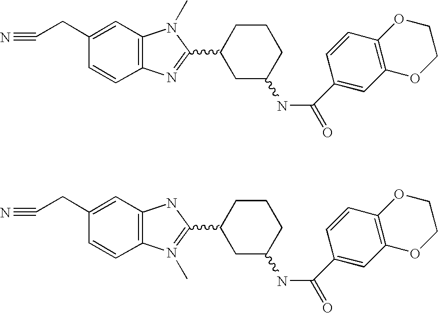

A 1:1 mixture of N-(3-(6-(chloromethyl)-1-methyl-1H-benzo[d]imidazol-2-yl)cyclohexyl)-2,3-dihydrobenzo-[b][1,4]dioxine-6-carboxamide and N-(3-(5-(chloromethyl)-1-methyl-1H-benzo[d]imidazol-2-yl)cyclohexyl)-2,3-dihydrobenzo(b][1,4]dioxine-6-carboxamide (0.21 mmol), was stirred with sodium cyanide (41 mg, 0.84 mmol), and DMF (2 ml) at +50° C. for four hours. The title products were isolated as a 1:1 mixture (22 mg) by chromatography on a silica gel column, eluting with a gradient from 0 to 5% of methanol in ethyl acetate. HPLC Rt=1.5; MS: [M+H]=431.1. SMO % inhibition at 2 uM=106.

### Example 36

### N-(3-(6-(2-Cyanopropan-2-yl)-1-methyl-1H-benzo[d]imidazol-2-yl)cyclohexyl)-2,3-dihydrobenzo[b)[1,4]dioxine-6-carboxamide and N-(3-(5-(2-cyanopropan-2-yl)-1-methyl-1H-benzo[d]imidazol-2-yl)cyclohexyl)-2,3-dihydrobenzo[b][1,4]dioxine-6-carboxamide

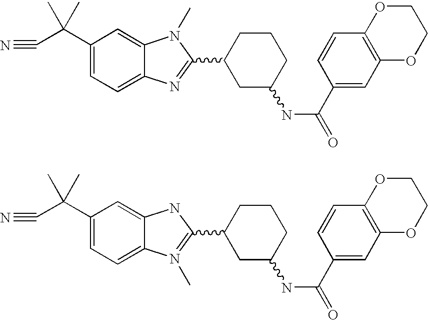

A 1:1 mixture of N-(3-(6-(cyanomethyl)-1-methyl-1H-benzo[d]imidazol-2-yl)cyclohexyl)-2,3-dihydrobenzo[b][1,4]dioxine-6-carboxamide and N-(3-(5-(cyanomethyl)-1-methyl-1H-benzo[d]imidazol-2-yl)cyclo hexyl)-2,3-dihydrobenzo[b][1,4]dioxine-6-carboxamide (Example 35, 20 mg, 0.046 mmol), was stirred with iodomethane (0.012 ml, 0.2 mmol), potassium hydroxide (11 mg, 0.2 mmol), water (0.1 ml), and DMSO (1 ml) at room temperature for three hours. After aqueous work-up, the title products were isolated as a 1:1 mixture (8 mg) by chromatography on a silica gel column, eluting with ethyl acetate. HPLC Rt=2.6; MS: [M+H]=459.1. SMO % inhibition at 2 uM=93.

### Example 37

### N-(3-(1-Methyl-6-(morpholinomethyl)-1H-benzo[d]imidazol-2-yl]cyclohexyl)-2,3-dihydrobenzo[b][1,4]dioxine-6-carboxamide and N-(3-(1-Methyl-5-(morpholinomethyl)-1H-benzo[d]imidazol-2-yl]cyclohexyl)-2,3-dihydrobenzo[b][1,4]dioxine-6-carboxamide

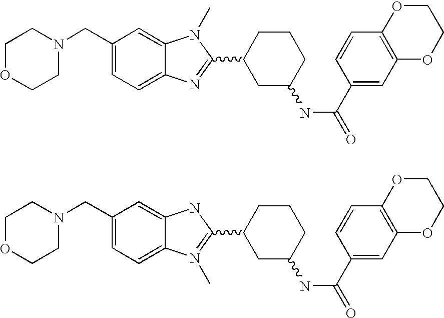

The title compounds were prepared according to procedure described in Example 34. HPLC Rt=1.8; MS: [M+H]=491.1. SMO % inhibition at 2 uM=88.

### Example 38

### N-(3-(6-((2-Methoxyethylamino)methyl)-1-methyl-1H-benzo[d]imidazol-2-yl)cyclohexyl)-2,3-dihydrobenzo[b][1,4]dioxine-6-carboxamide and N-(3-(5-((2-methoxyethylamino)methyl)-1-methyl-1H-benzo[d]imidazol-2-yl)cyclohexyl)-2,3-dihydrobenzo[b][1,4]dioxine-6-carboxamide

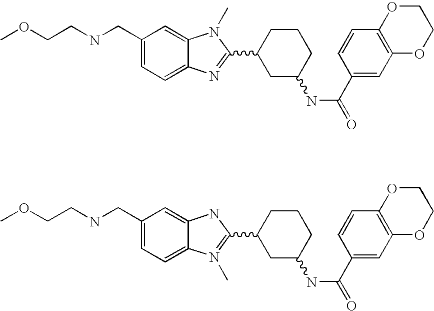

The title compounds were prepared according to procedure described in Example 34. HPLC Rt=1.8; MS: [M+H]=479.1. SMO % inhibition at 2 uM=103.

### Example 39

### N-(3-(6-(Dimethylamino)-1H-benzo[d]imidazol-2-yl)cyclohexyl-2,3-dihydrobenzo[d][1,4]dioxine-6-carboxamide

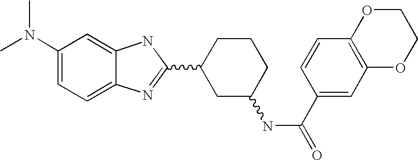

N¹,N¹-Dimethylbenzene-1,3,4-triamine. A mixture of N¹,N¹-dimethyl-4-nitrobenzene-1,3-diamine (200 mg, 1.1 mmol), Raney nickel (200 mg), and THF (20 ml) was shaken at room temperature under 30-40 psi of hydrogen gas for two hours. The mixture was filtered through a pad of Celite and concentrated to provide the target product, which must be used immediately for the next step.

N-(3-(6-(Dimethylamino)-1H-benzo[d]imidazol-2-yl)cyclohexyl-2,3-dihydrobenzo[d][1,4]dioxine-6-carboxamide. The title compound can be prepared from N¹,N¹-dimethylbenzene-1,3,4-triamine and 3-(2,3-dihydrobenzo[b][1,4]dioxine-7-carboxamido)-cyclohexanecarboxylic acid similarly to the procedure from Example 28. HPLC Rt 2.2; MS: [M+H]=421.1. SMO % inhibition at 2 uM=102.

### Example 40

### N-[(1R,3S)-3-(1,6-Dimethyl-1H-benzo[d]imidazol-2-yl)cyclohexyl)-2,3-dihydrobenzo[b][1,4]dioxine-6-carboxamide

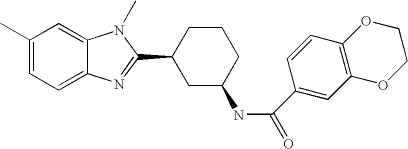

N,5-Dimethyl-2-nitrobenzenamine. A mixture of 2-fluoro-4-methyl-1-nitrobenzene (2.55 g, 16.4 mmol), 33% solution of methylamine in ethanol (5 ml), and ethanol (15 ml) was stirred at 80° for two hours. Water (50 ml) was added, and the target product (2.4 g) was isolated as an orange solid by filtration.

N¹,5-Dimethylbenzene-1,2-diamine. A mixture of N,5-dimethyl-2-nitrobenzenamine (2.4 g), 10% palladium on activated carbon (100 mg), and methanol (60 ml) was shaken at room temperature under 30-40 psi of hydrogen gas for one hour. The mixture was filtered through a pad of Celite and concentrated to provide the target product.

N-(3-(1,6-Dimethyl-1H-benzo[d]imidazol-2-yl)cyclohexyl)-2,3-dihydrobenzo[b][1,4]dioxine-6-carboxamide. The title product was obtained according to Example 28.

### Example 41

### N-((1R,3S)-3-(1H-Benzo-[d]imidazol-2-yl)cyclohexyl)-6-(2-methoxyethylamino)nicotinamide

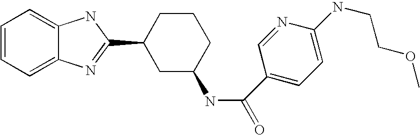

N-((1R,3S)-3-(1H-Benzo[d]imidazol-2-yl)cyclohexyl)-6-chloronicotinamide. This compounds was obtained according to Example 4. HPLC Rt=1.3; MS: [M+H]=394.3. SMO % inhibition at 2 uM=103.

N-((1R,3S)-3-(1H-Benzo-[d]imidazol-2-yl)cyclohexyl)-6-(2-methoxyethylamino)nicotinamide. A mixture of N-((1R,3S)-3-(1H-benzo[d]imidazol-2-yl)cyclohexyl)-6-chloronicotinamide (21 mg), 2-methoxyethylamine (0.1 ml), and NMP (0.8 ml) was stirred at 130° C. for 24 hours. The title product (11 mg) was isolated by chromatography on reverse phase column, eluting with a gradient from 5 to 50% of acetonitrile in 0.1% aqueous formic acid. HPLC Rt=1.3; MS: [M+H]=394.3. SMO % inhibition at 2 uM=87.

### Example 42

### N-((1R,3S)-3-(1H-Benzo[d]imidazol-2-yl)cyclohexyl)-2-(2-methoxyethylamino)isonicotinamide

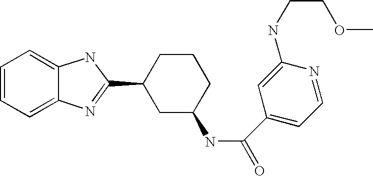

The title compound was prepared in a manner similar to Example 41. HPLC Rt=1.3; MS: [M+H]=394.3. SMO % inhibition at 2 uM=87.

### Example 43

### N-((1R,3S)-3-(5-(2-Methoxyethylamino)methyl)-1H-benzo[d]imidazol-2-yl)cyclohexyl)-2,3-dihydrobenzo[b][1,4]dioxine-6-carboxamide

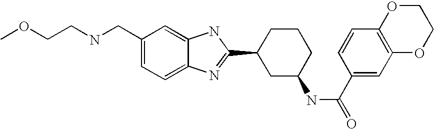

N-((1R,3S)-3-(6-Formyl-1H-benzo[d]imidazol-2-yl)cyclohexyl)-2,3-dihydrobenzo[b][1,4]dioxine-6-carboxamide. To a stirred mixture of N-((1R,3S)-3-(6-cyano-1H-benzo[d]imidazol-2-yl)cyclohexyl)-2,3-dihydrobenzo[b][1,4]dioxine-6-carboxamide (prepared similarly to Example 4, 150 mg, 0.37 mmol) and DCM (3 ml) a 1.0 M solution of DIBAL-H in DCM (2.0 ml, 2.0 mmol) was added dropwise at 0° C., and the obtained solution was stirred at room temperature for one hour. THF (10 ml) and water (0.5 ml) were added, the formed precipitate was filtered off, the mother liquor was concentrated and chromatography on a silica gel column, eluting with a gradient from 70 to 100% ethyl acetate in heptane, provided 50 mg of the target product.

N-((1R,3S)-3-(5-(2-Methoxyethylamino)methyl)-1H-benzo[d]imidazol-2-yl)cyclohexyl)-2,3-dihydrobenzo[b][1,4]dioxine-6-carboxamide. To a stirred solution of N-((1R,3S)-3-(6-formyl-1H-benzo[d]imidazol-2-yl)cyclohexyl)-2,3-dihydrobenzo[b][1,4]dioxine-6-carboxamide (17 mg, 0.04 mmol) and 2-methoxyethylamine (0.2 mmol) in 0.8 ml of DCM a solution of sodium triacetoxyborohydride (25 mg, 0.12 mmol) in DCM (0.9 ml) was added at room temperature in one portion, followed by acetic acid (0.08 ml). After two hours the reaction mixture was concentrated and subjected to chromatography on a reverse phase column, eluting with a gradient from 5 to 50% of acetonitrile in 0.1% aqueous formic acid to obtain 3.4 mg of the title product. HPLC Rt=1.8; MS: [M+H]=465.2. SMO % inhibition at 2 uM=99.

### Example 44

### N-((1R,3S)-3-(5-((Dimethylamino)methyl)-1H-benzo[d]imidazol-2-yl)cyclohexyl)-2,3-dihydrobenzo[b][1,4]dioxine-6-carboxamide

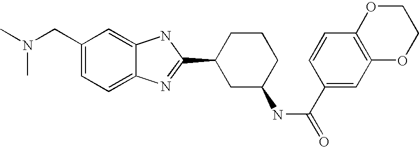

The title compound was prepared in a fashion similar to Example 43. HPLC Rt=1.6; MS: [M+H]=435.2. SMO % inhibition at 2 uM=107.

### Example 45

### N-((1R,3S)-3-(1H-Benzo[d]midazol-2-yl)cyclohexyl)-4-methyl-3-oxo-3,4-dihydro-2H-benzo[b][1,4]oxazine-6-carboxamide

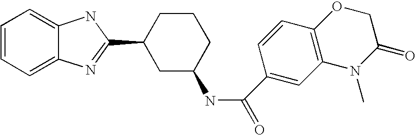

Methyl 4-methyl-3-oxo-3,4-dihydro-2H-benzo[b][1,4]oxazine-6-carboxylate. A mixture of 3-oxo-3,4-dihydro-2H-benzo[b][1,4]oxazine-6-carboxylic acid (200 mg, 1.04 mmol), iodomethane (1.4 ml, 22 mmol), silver (I) oxide (980 mg, 4.23 mmol), and DMF (5 ml) was stirred at room temperature for two days. Ethyl acetate was added, the mixture was filtered through Celite, and the mother liquor was washed with water, brine, dried over magnesium sulfate, and concentrated to obtain the target product.

4-Methyl-3-oxo-3,4-dihydro-2H-benzo[b][1,4]oxazine-6-carboxylic acid. A mixture of methyl 4-methyl-3-oxo-3,4-dihydro-2H-benzo[b][1,4]oxazine-6-carboxylate (220 mg, 0.99 mmol), lithium hydroxide hydrate (63 mg, 1.5 mmol), THF (4.5 ml), and water (1.5) was stirred at room temperature for two days. The mixture was acidified with hydrochloric acid and extracted with ethyl acetate. The extract was dried over magnesium sulfate and concentrated to obtain the target product.

N-((1R,3S)-3-(1H-Benzo[d]midazol-2-yl)cyclohexyl)-4-methyl-3-oxo-3,4-dihydro-2H-benzo[b][1,4]oxazine-6-carboxamide. A mixture of 4-methyl-3-oxo-3,4-dihydro-2H-benzo[b][1,4]oxazine-6-carboxylic acid (23 mg, 0.11 mmol), (1R,3S)-3-(1H-benzo[d]imidazol-2-yl)cyclohexanamine (Example 1, 29 mg, 0.10 mmol), benzotriazol-1-yloxy)tris(dimethylamino)phosphonium hexafluorophosphate (49 mg, 0.11 mmol), diisopropylethylamine (0.053 ml, 0.30 mmol), and DMF (1 ml) was stirred at room temperature for 18 hours. The reaction mixture was poured into 5 ml of 2% aqueous sodium bicarbonate. The obtained mixture was extracted with ethyl acetate. The extract was dried over magnesium sulfate. Chromatography on a silica gel column, eluting with a gradient from 70 to 100% ethyl acetate in heptane yielded 7 mg of the title compound. 1H NMR (methanol-d4, 400 MHz) 1.4-1.5 (m, 1H), 1.55-1.65 (m, 2H), 1.65-1.75 (m, 1H), 1.95-2.1 (m, 3 H), 2.3-2.35 (m, 1H), 3.0-3.1 (m, 1H), 3.35 (3H), 4.0-4.1 (m, 1H), 4.65 (s, 2H), 7.0 (d, 1H), 7.15-7.2 (m, 2H), 7.45-7.5 (m, 2H), 7.55 (d, 1H), 7.6 (s, 1H). HPLC Rt=1.9. MS: [M+H]=405.6. SMO % inhibition at 2 uM=108.

### Example 46

### N-(3-(1H-Benzo[d]imidazol-2-yl)cyclohexyl)-2-(6-chloro-1H-benzo[d]imidazol-2-yl)acetamide

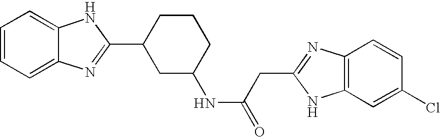

A mixture of 2-(6-chloro-1H-benzo[d]imidazol-2-yl)acetic acid (252 mg (1.2 mmol), HBTU (455 mg (1.2 mmol), DIPEA (0.78 ml, 4.5 mmol), and DMF (6 ml) was stirred at room temperature for 30 minutes. The obtained clear solution was added to 3-(1H-benzo[d]imidazol-2-yl)cyclohexanamine dihydrochioride (Example 1, 314 mg, 1.09 mmol) and the resulting mixture was stirred at room temperature for three days. The mixture was poured into 70 ml of 2% aqueous sodium bicarbonate. Extraction with ethyl acetate and chromatography on a silica gel column, eluting with a gradient from 3 to 10% methanol in ethyl acetate, provided 444 mg (64%) of the title compound. HPLC Rt=1.2. MS: [M+H]=408.0. SMO % inhibition at 2 uM=108.

### Example 47

### 1-((1R,3S)-3-(1H-Benzo[d]imidazol-2-yl)cyclohexyl]-3-(4-(trifluoromethyl)phenyl)urea

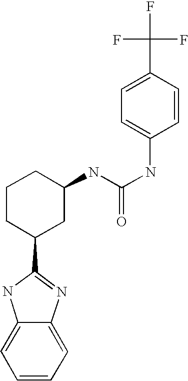

1-Isocyanato-4-(trifluoromethyl)benzene (0.3 mmol) was added to a solution of (1R,3S)-3-(1H-benzo[d]imidazol-2-yl)cyclohexanamine (0.2 mmol) and triethylamine (0.3 mmol) in DMF (1 mL) at room temperature. The reaction mixture was stirred at room temperature for 18 hours. The reaction mixture was subjected to chromatography on a reverse phase column, eluting with a gradient from 15 to 50% of acetonitrile in 0.1% aqueous formic acid to obtain 53 mg of the title product as a white solid. HPLC Rt=1.6. MS: [M+H]=376.6. SMO % inhibition at 2 uM=108.

### Example 48

### N-((1R,3R)-3-(1H-benzo[d]imidazol-2-yl)cyclohexyl)-3-oxo-3,4-dihydro-2H-benzo[b][1,4]oxazine-6-carboxamide and N-((1S,3S)-3-(1H-benzo[d]imidazol-2-yl)cyclohexyl)-3-oxo-3,4-dihydro-2H-benzo[b][1,4]oxazine-6-carboxamide

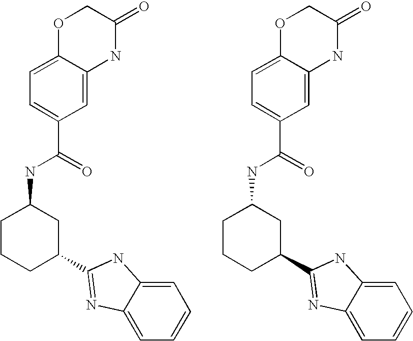

(1S,4S,5S)-4-Iodo-6-oxa-bicyclo[3.2.1]octan-7-one and (1R,4R,5R)-4-iodo-oxa-bicyclo[3.2.1]octan-7-one. To a stirred mixture of racemic cyclohex-3-enecarboxylic acid (5 g, 39.6 mmol), sodium bicarbonate (10 g, 119 mmol), and water (100 ml) a solution of iodine (10.7 g, 42.1 mmol) and potassium iodide (39.7 g, 239.2 mmol) in water (100 ml) was added in one portion at room temperature. The flask was immediately covered with aluminum foil and the resulting mixture was stirred at room temperature for three days. The desired product (9.2 g of a racemic (1:1) mixture) was collected by filtration as a white solid.

(1S,5S)-6-Oxa-bicyclo[3.2.1]oct-3-en-7-one and (1 R,5R)-6-oxa-bicyclo[3.2.1]oct-3-en-7-one. A mixture of (1S,4S,5S)-4-iodo-6-oxa-bicyclo[3.2.1]octan-7-one and (1R,4R,5R)-4-iodo-6-oxa-bicyclo[3.2. 1]octan-7-one (9.2 g, 36.5 mmol), DBU (8.34 g, 54.8 mmol), and toluene (120 ml) was stirred at +120° C. for 90 minutes. The mixture was washed with 1M hydrochloric acid, brine, dried over magnesium sulfate and concentrated in vacuum to provide 3.2 g of the desired product as a racemic (1:1) mixture.

(1S,5R)-5-Azidocyclohex-3-enecarboxylic acid and (1R,5S)-5-azidocyclohex-3-enecarboxylic acid. A mixture of (1S,5S)-6-oxa-bicyclo[3.2.1]oct-3-en-7-one and (1R,5R)-6-oxa-bicyclo[3.2.1]oct-3-en-7-one (3.2 g, 25.8 mmol), sodium azide (1.84 g, 28.4 mmol), THF (50 ml), and water (20 ml) was stirred at +50° C. for two days. After concentration in vacuum, 40 ml of water was added, and the mixture was washed with MTBE (20 ml), acidified with 1 M hydrochloric acid, and extracted with ethyl acetate. The organic extract was dried over magnesium sulfate and concentrated in vacuum to provide 2.8 g of the desired product as a racemic (1:1) mixture.

(1S,3S)-Methyl 3-aminocyclohexanecarboxylate and (1R,3R)-methyl 3-aminocyclohexane-carboxylate hydrochloride. A mixture of (1S,5R)-5-azidocyclohex-3-enecarboxylic acid and (1R,5S)-5-azidocyclohex-3-enecarboxylic acid (2.8 g, 16.8 mmol), 10% palladium on activated carbon (200 mg), and methanol (30 ml) was shaken under 30-40 psi of hydrogen gas at room temperature for four hours. The obtained mixture was saturated with hydrogen chloride and stirred at room temperature for, filtered through a pad of Celite, and concentrated to give 2.16 g of the target product as a racemic (1:1) mixture.

(1S,3S)-Methyl 3-(benzyloxycarbonyl)cyclohexanecarboxylate and (1R,3R)-methyl 3-(benzyloxycarbonyl)cyclohexanecarboxylate. A mixture of (1S,3S)-methyl 3-aminocyclohexane-carboxylate and (1R,3R)-methyl 3-aminocyclohexanecarboxylate hydrochloride (2.16 g, 11.2 mmol), benzyl chloroformate (1.9 ml, 13.4 mmol), powdered sodium carbonate (3.55 g, 33.5 mmol), and acetonitrile (40 ml) was stirred at room temperature for three days. The mixture was loaded on silica gel and chromatographed on a silica gel column, eluting with a gradient from 20 to 40% ethyl acetate in hepatne to obtain 2.7 g of the desired product as a colorless oil (1:1 racemic mixture).

(1S,3S)-3-(Benzyloxycarbonyl)cyclohexanecarboxylic acid and (1R,3R)-3-(benzyloxycarbonyl)-cyclohexanecarboxylic acid. A mixture of (1S,3S)-methyl 3-(benzyloxycarbonyl)cyclohexanecarboxylate and (1R,3R)-methyl 3-(benzyloxycarbonyl)cyclohexanecarboxylate (1:1, 2.7 g, 9.3 mmol), lithium hydroxide hydrate (0.58 g, 13.9 mmol), THF (45 ml), and water (15 ml) was stirred at room temperature for 18 hours. The mixture was acidified with 6 M hydrochloric acid, diluted with 30 ml of brine, and extracted with ethyl acetate. The extract was dried over magnesium sulfate and concentrated in vacuum to provide 2.25 g of the desired product as a racemic mixture (1:1).

N-((1R,3R)-3-(1H-benzo[d]imidazol-2-yl)cyclohexyl)-3-oxo-3,4-dihydro-2H-benzo[b][1,4]oxazine-6-carboxamide and N-((1S,3S)-3-(1H-benzo[d]imidazol-2-yl)cyclohexyl)-3-oxo-3,4-dihydro-2H-benzo[b][1,4]oxazine-6-carboxamide. The title compounds were prepared as a racemic (1:1) mixture according to the procedure described in Example 4. HPLC Rt=1.6; MS: [M+H]=391.2. SMO % inhibition at 2 uM=80.

### Example 49

### N-((1R,3R)-3-(5-Methoxy-1H-benzo[d]imidazol-2-yl)cyclohexyl)-2,3-dihydrobenzo[b][1,4]dioxine-6-carboxamide and N-((1S,3S)-3-(5-methoxy-1H-benzo[d]imidazol-2-yl)cyclohexyl)-2,3-dihydrobenzo[b][1,4]dioxine-6-carboxamide

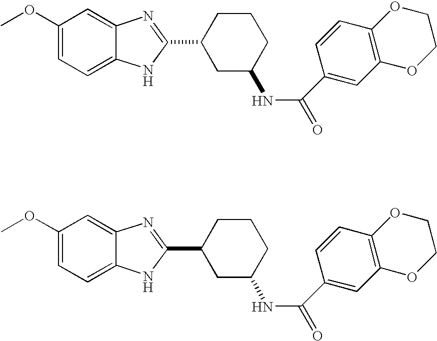

The title compounds were prepared as a racemic (1:1) mixture from (1S,3S)-3-(benzyloxycarbonyl)-cyclohexanecarboxylic acid and (1R,3R)-3-(benzyloxycarbonyl)cyclohexanecarboxylic acid racemic mixture (Example 48) and 4-methoxybenzene-1,2-diamine according to the procedure described in Example 4. HPLC Rt=1.3. MS: [M+H]=408.1. SMO % inhibition at 2 uM=92.

### Example 50

### N-[(1R,3S)-3-(1H-benzimidazol-2-yl)cyclohexyl]-3,4-dihydro-2H-1,5-benzodioxepine-7-carboxamide

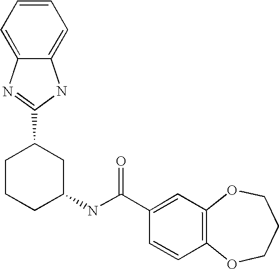

A mixture of 3,4-dihydro-2H-benzo[b][1,4]dioxepine-7-carboxylic acid (0.10 mmol), (1R,3S)-3-(1H-benzo[d]imidazol-2-yl)cyclohexanamine (Example 1, 0.10 mmol), HATU (0.10 mmol), triethylamine (0.10 mmol) in DMF (1.4 ml) was shaken at room temperature for 16 hours. The reaction mixture was evaporated to dryness and the product purified by reverse phase HPLC. LC MS M⁺+1 392.092, retention time: 1.12 min.

### Example 51

### N-[5-(1H-benzimidazol-2-yl)bicyclo[3.1.1]hept-1-yl]-2,3-dihydro-1,4-benzodioxine-6-carboxamide

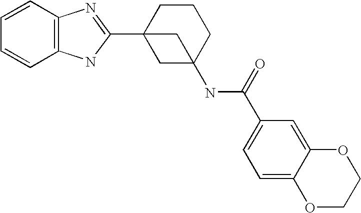

Bicyclo[3.1.1]heptane-1,5-dicarboxylic acid dimethyl ester. Prepared as described by Warner et al. (J. Org. Chem. 1981, 46, 4795).

Bicyclo[3.1.1]heptane-1,5-dicarboxylic acid monomethyl ester. Solid Ba(OH)₂ (9.66 g, 30.64 mmol) was added portion wise to a solution of bicyclo[3.1.1]heptane-1,5-dicarboxylic acid dimethyl ester (13 g, 61.29 mmol) in 80% aqueous MeOH (156 ml) at 0° C. The reaction mixture was stirred for overnight, evaporated to remove the alcohol. The crude residue was diluted with water, washed with pentane and then acidified with conc. HCl (pH-3). It was extracted with ethyl acetate, dried over sodium sulfate and evaporated to afford the desired product (8 g, 66%) as an off white solid.

5-tert-Butoxycarbonylamino-bicyclo[3.1.1]heptane-1-carboxylic acid methyl ester. Triethyl amine (2.05 ml, 7.57 mmol) was added to a solution of [3.1.1]heptane-1,5-dicarboxylic acid monomethyl ester (1.5 g, 7.57 mmol) in benzene (31 ml) followed by DPPA (2.08 g, 7.57 mmol) under argon atmosphere. It was refluxed for 45 min, cooled to RT and t-BuOH (1.43 ml, 15.14 mmol) was added drop wise and again refluxed for overnight. The reaction mass was cooled, evaporated and the crude residue was purified by column chromatography over silica gel by eluting with 5% EtOAc in hexane to the desired product (0.9 g, 45%) as light yellow liquid.

¹H NMR (400 MHz, CDCl₃): δ 3.65 (s, 3H), 2.46 (br s, 2H), 1.94-1.84 (m, 8H), 1.42 (s, 9H). FIA-MS (M+H)⁺: 213.3.

5-tert-Butoxycarbonylamino-bicyclo[3.1.1]heptane-1-carboxylic acid. Solid LiOH (2.18 g, 51.98 mmol) was added portion wise to a solution of 5-tert-Butoxycarbonylamino-bicyclo[3.1.1]heptane-1-carboxylic acid methyl ester (3.5 g, 12.09 mmol) in 80% aqueous MeOH (14 ml) at 0° C. The reaction mixture was stirred for 48 h, evaporated to remove the alcohol. The crude residue was diluted with water, washed with pentane and then acidified with 10% citric acid (pH-5). It was extracted with dichloromethane, dried over sodium sulfate and evaporated to afford the desired product (2.8 g, 85%) as an off white solid.

¹H NMR (400 MHz, DMSO-d₆): δ 12.2 (br s, 1H), 6.97 (br s, 1H), 2.29 (br m, 2H), 1.78-1.75 (m, 8H), 1.37 (s, 9H).

2,3-Dihydro-benzo[1,4]dioxine-6-carboxylic acid [5-(1H-benzoimidazol-2-yl)-bicyclo[3.1.1]hept-1yl]-amide. To a solution of 5-tert-Butoxycarbonylamino-bicyclo[3.1.1]heptane-1-carboxylic acid (1.522 g, 6.00 mmol) and phenylene diamine (0.865 g, 8.00 mmol) in DMF (30 ml) was added triethyl amine (0.809 g, 8.00 mmol) and HATU (2.281 g, 6.00 mmol). The resulting mixture was stirred at 70° C. for 18 hours. LCMS indicated a 9:1 mixture of the intermediate [5-(2-Amino-phenylcarbamoyl)-bicyclo[3.1.1]hept-1-yl]-carbamic acid tert-butyl ester and intermediate [5-(1H-Benzoimidazol-2-yl)-bicyclo[3.1.1]hept-1-yl]-carbamic acid tert-butyl ester. Solvents were evaporated and the crude residue was treated with 4M HCl in 1,4-dioxane (20 ml) at 100° C. for 18 hours. The crude reaction was cooled to room temperature, diluted with methanol (40 mL) and brought to pH 8 by the drop wise addition of conc. ammonium hydroxide. The crude mixture was adsorbed on to silica gel and purified by column chromatography to afford the desired product (1.238 g, 91%) as an off white solid.

¹H NMR (400 MHz, DMSO-d₆): δ 7.4 (m, 2H), 7.1 (m, 2H), 2.5 (m, 3H), 2.06 (m, 3H), 1.9 (m, 4 H)

FIA/MS AP+ 228.2, AP− 226.2

2,3-Dihydro-benzo[1,4]dioxine-6-carboxylic acid [5-(1H-benzoimidazol-2-yl)-bicyclo[3.1.1]hept-1yl]-amide. To a mixture of 2,3-Dihydro-benzo[1,4]dioxine-6-carboxylic acid [5-(1H-benzoimidazol-2-yl)-bicyclo[3.1.1]hept-1yl]-amide (0.455 g, 2.0 mmol) and triethy amine (0.61 g, 6.0 mmol) in acetonitrile (20 ml) was added 2,3-Dihydro-benzo[1,4]dioxine-6-carbonyl chloride (0.438 g, 2.2 mmol) and the resulting mixture was stirred at room temperature overnight. The reaction mixture was diluted with methanol (20 ml) adsorbed on to silica gel and purified by column chromatography to afford the final product (0.312 g, 40%).

2,3-Dihydro-benzo[1,4]dioxine-6-carboxylic acid [5-(1H-benzoimidazol-2-yl)-bicyclo[3.1.1]hept-1yl]-amide was taken up in methanol. HCl and dioxane were added dropwise. The solution was concentrated and vacuum-dried, yielding the HCl salt of the title compound.

¹H NMR (400 MHz, DMSO-d₆): δ 8.45 (s, 1H), 7.42 (br, 1H), 7.35 (m, 3H), 7.06 (m, 2H), 6.86 (d, 1H), 4.23 (s, 4H), 2.45 (m,2H), 2.23 (m, 2H), 2.05 (m,4H), 1.92 (m, 2H).

LCMS: RT=2.0 min.; ES+ 390.2; ES− 388.3.

Examples listed in the following table were prepared using procedures analogous to those described above. In the following table, the structures are shown; if a salt is associated it is identified in the “Compound Name” column.

| | | | ANALYTICAL | SMO CELL % | |
|---|---|---|---|---|---|
| EXAMPLE NO. | COMPOUND NAME | STRUCTURE | DATA | INHIBITION @ 2 μM | EXAMPLE # PREP |
| 52 | N-[3-(1H- benzimidazol-2- yl)cyclohexyl]- 3,5-dimethoxybenz- amide HCl | 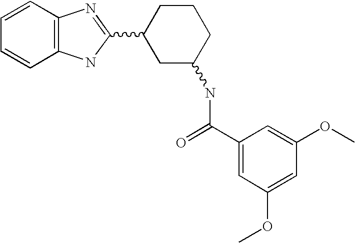 | HPLC Rt = 1.48, MS: [M + H] = 380 | 105 | 1 |
| 53 | N-[3-(1H- benzimidazol-2- yl)cyclohexyl]- nicotinamide HCl | 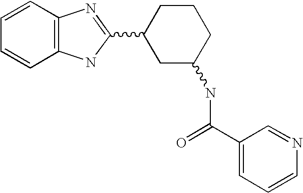 | HPLC Rt = 0.79, MS: [M + H] = 321 | 96 | 1 |
| 54 | N-[3-(1H- benzimidazol-2- yl)cyclohexyl]- 3- cyclopentylpro- panamide HCl | 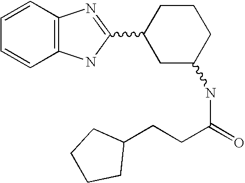 | HPLC Rt = 1.6 MS: [M + H] = 340 | 67 | 45 |
| 55 | N-[3-(1H- benzimidazol-2- yl)cyclohexyl]- 2,2- dimethylpropan- amide HCl | 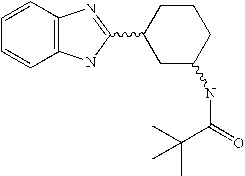 | HPLC Rt = 1.17 MS: [M + H] = 300 | 57 | 45 |
| 56 | N-[3-(1H- benzimidazol-2- yl)cyclohexyl]- 2-furamide HCl | 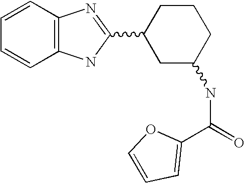 | HPLC Rt = 0.9 MS: [M + H] = 310 | 51 | 45 |
| 57 | N-(3-(1H- benzo[d]imidazol- 2- yl)cyclohexyl) cyclohexanecar- boxamide HCl | 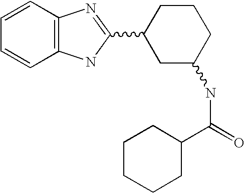 | HPLC Rt = 1.3 MS: [M + H] = 326 | 48 | 1 |
| 58 | N-[3-(1H- benzimidazol-2- yl)cyclohexyl]- 1,3- benzodioxole- 5-carboxamide HCl | 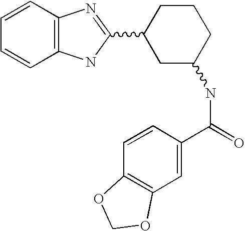 | HPLC Rt = 1.3 MS: [M + H] = 364 | 82 | 1 |
| 59 | N-[3-(1H- benzimidazol-2- yl)cyclohexyl]- 3-chloro-4- methoxybenz- amide HCl | 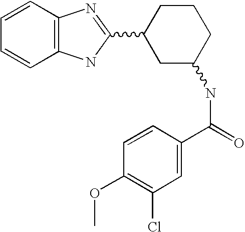 | HPLC Rt = 1.6 MS: [M + H] = 384 | 101 | 1 |
| 60 | N-[3-(1H- benzimidazol-2- yl)cyclohexyl]- 1H- benzimidazole- 5-carboxamide HCl | 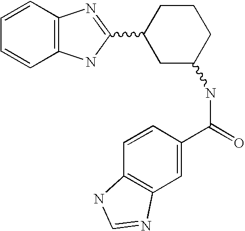 | HPLC Rt = 0.5 MS: [M + H] = 360 | 87 | 45 |
| 61 | N-[3-(1H- benzimidazol-2- yl)cyclohexyl]- 1H-imidazole-2- carboxamide HCl | 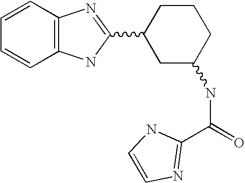 | HPLC Rt = 0.8 MS: [M + H] = 310 | 52 | 45 |
| 62 | N-[3-(1H- benzimidazol-2- yl)cyclohexyl]- 4- methoxybenz- amide HCl | 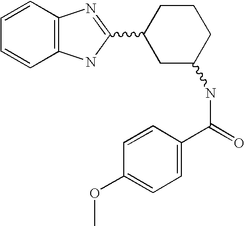 | HPLC Rt = 1.3 MS: [M + H] = 350 | 72 | 1 |
| 63 | N-[(1R,3S)-3- (1H- benzimidazol-2- yl)cyclohexyl]- 2,3-dihydro-1,4- benzodioxine- 6-carboxamide HCl | 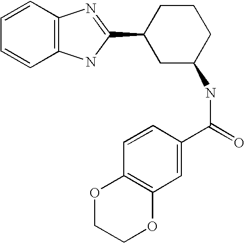 | HPLC Rt = 1.3 MS: [M + H] = 378 | 108 | 2 |
| 64 | N-[(1R,3S)-3- (1H- benzimidazol-2- yl)cyclohexyl]- 2,3-dihydro-1,4- benzodioxine- 6-carboxamide triflouroacetate | 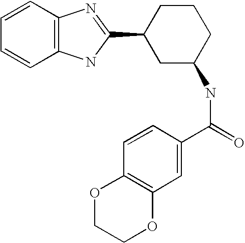 | HPLC Rt = 1.3 MS: [M + H] = 378 | 108 | 2 |
| 65 | N-[(1R,3R)-3- (1H- benzimidazol-2- yl)cyclohexyl]- 2,3-dihydro-1,4- benzodioxine- 6-carboxamide HCl | 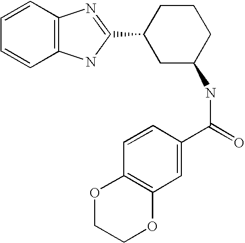 | 1H NMR (CD3OD) 1.75 (m, 4H), 2.0 (m, 2H), 2.2 (m, 2H), 3.4 (t, 1H), 4.2 (m, 1H), 4.25 (m, 4H), 6.85 (d, 1H), 7.25 (m, 2H), 7.35 (m, 2H), and 7.55 (m, 2H); HPLC Rt = 1.61 MS: [M + H] = 378 | 96 | 2 |
| 66 | N-[(1R,3S)-3- (1-methyl-1H- benzimidazol-2- yl)cyclohexyl]- 2,3-dihydro-1,4- benzodioxine- 6-carboxamide HCl | 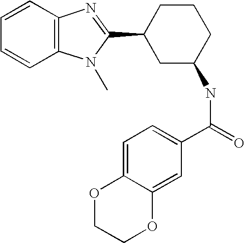 | HPLC Rt = 1.55 MS: [M + H] = 392 | 107 | 3 |
| 67 | N-[(1R,3S)-3- (1-methyl-1H- benzimidazol-2- yl)cyclohexyl]- 2,3-dihydro-1,4- benzodioxine- 6-carboxamide (+)-tartrate | 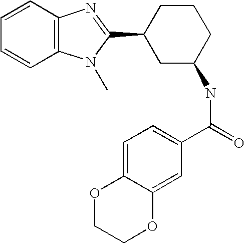 | HPLC Rt = 1.55 MS: [M + H] = 392 | 107 | 3 |
| 68 | N-[3-(6-cyano- 1H- benzimidazol-2- yl)cyclohexyl]- 3,5-dimethoxybenz- amide | 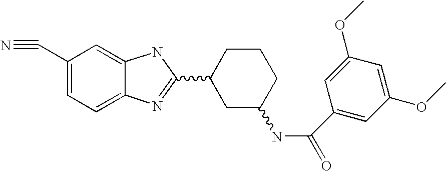 | HPLC Rt = 2.8 MS: [M + H] = 405.3 | 89 | 28 |
| 69 | 3,5-dimethoxy- N-[3-(6- methoxy-1H- benzimidazol-2- yl)cyclohexyl] benzamide | 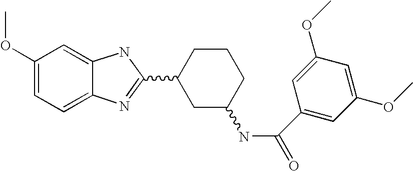 | HPLC Rt = 2.5 MS: [M + H] = 410.3 | 105 | 28 |
| 70 | N-[3-(6-fluoro- 1H- benzimidazol-2- yl)cyclohexyl]- 3,5- dimethoxybenz- amide | 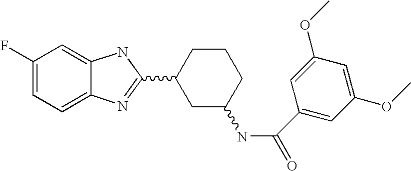 | 1H NMR (d6- acetone) (methanol- d4, 400 MHz), 1.4-1.5 (m, 1H), 1.55-1.65 (m, 1H), 1.65- 1.75 (m, 2H), 1.95-2.1 (m, 4H), 3.0- 3.1 (m, 1H), 3.75 (s, 6H), 4.0-4.1 (m, 1H), 6.6 (s, 1H), 6.95 (s, 2H), 6.9-6.95 (m,1 H), 7.15 (d, 1H), 7.4-7.45 (m, 1H); HPLC Rt = 2.6 MS: [M + H] = 398.3 | 83 | 28 |
| 71 | N-{3-[1-(2- hydroxyethyl)- 1H- benzimidazol-2- yl]cyclohexyl}- 2,3-dihydro-1,4- benzodioxine- 6-carboxamide | 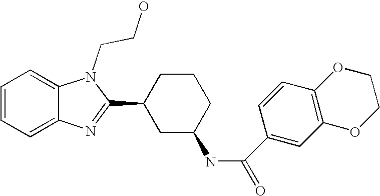 | HPLC Rt = 2 MS: [M + H] = 422.3 | 98 | 13 |
| 72 | N-((1S,3R)-3- (1H- benzo[d]imidazol- 2- yl)cyclohexyl)- 2,3- dihydrobenzo[b] [1,4]dioxine-6- carboxamide HCl | 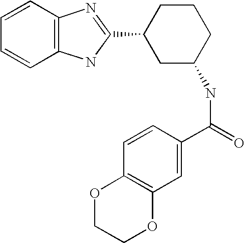 | HPLC Rt = 2 MS: [M + H] = 378.1 | 96 | 1 |
| 73 | N-{3-[5- (aminomethyl)- 1-methyl-1H- benzimidazol-2- yl]cyclohexyl}- 2,3-dihydro-1,4- benzodioxine- 6-carboxamide HCl | 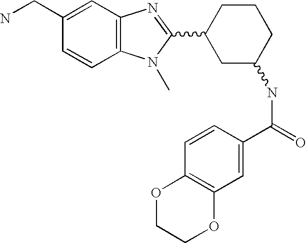 | HPLC Rt = 2.6 MS: [M + H] = 421.1 | 104 | 23 |
| 74 | 3,5-dimethoxy- N-{3-[6- (trifluoromethyl)- 1H- benzimidazol-2- yl)cyclohexyl}- benzamide HCl | 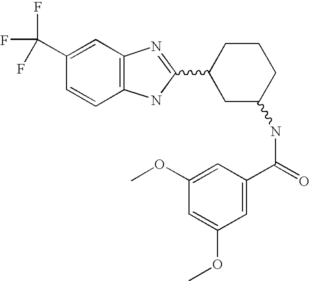 | HPLC Rt = 2.8 MS: [M + H] = 448.1 | 105 | 24 |
| 75 | N-((1R,3S)-3- (1H- benzo[d]imidazol- 2- yl)cyclohexyl)- 2,3- dihydrobenzo[b] [1,4]dioxine-6- carbothioamide HCl | 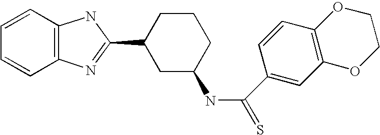 | HPLC Rt = 1.6 MS: [M + H] = 394 | 104 | 52 |
| 76 | N-{(1R,3S)-3- [1-(2- methoxyethyl)- 1H- benzimidazol-2- yl]cyclohexyl}- 2,3-dihydro-1,4- benzodioxine- 6-carboxamide | 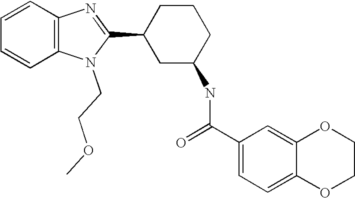 | 1H NMR (d6- acetone) 1.5 (m, 2H), 1.7 (m, 1H), 1.9-2.0 (m, 3H), 2.2 (n, 1H), 2.9 (m, 1H), 3.2 (s, 3H), 3.3 (m, 1H), 3.7 (t, 2H), 4.15 (m, 1H), 4.27 (m, 4H), 4.4 (t, 2H), 6.83 (m, 1H), 7.17 (m, 2H), 7.43 (m, 3H), 7.6 (m, 1H), 8.1 (broad, 1H); HPLC Rt = 1.6 MS: [M + H] = 436.1 | 101 | 16 |
| 77 | N-{3-[6- (dimethylamino)- 1H- benzimidazol-2- yl]cyclohexyl}- 2,3-dihydro-1,4- benzodioxine- 6-carboxamide HCl | 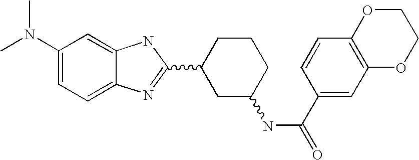 | HPLC Rt = 2.2 MS: [M + H] = 421.1 | 102 | 39 |
| 78 | N-[3-(6-tert- butyl-1H- benzimidazol-2- yl)cyclohexyl]- 2,3-dihydro-1,4- benzodioxine- 6-carboxamide HCl | 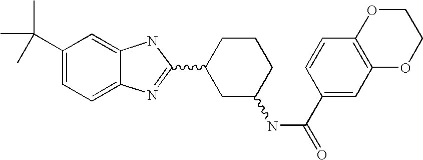 | HPLC Rt = 2.1 MS: [M + H] = 434.1 | 100 | 28 |
| 79 | N-[3-(7-methyl- 1H- benzimidazol-2- yl)cyclohexyl]- 2,3-dihydro-1,4- benzodioxine- 6-carboxamide HCl | 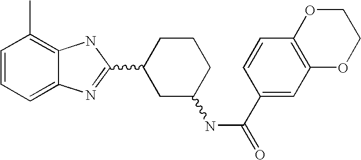 | 1H NMR (d6- acetone) (methanol- d4, 400 MHz) 1.5-1.6 (m, 1H), 1.65-1.8 (m, 3H), 2.0-2.1 (m, 2H), 2.15-2.2 (m, 1H), 2.4- 2.45 (m, 1H), 2.6 (s, 3H), 3.25-3.4 (m, 1H), 3.8 (s, 3H), 4.0- 4.1 (m, 1H), 4.2 (s, 4H), 4.7 (s, 2H), 6.85 (d, 1H), 7.3-7.35 (m, 3H), 7.4- 7.45 (m, 1H), 7.5 (d, 1H); HPLC Rt= 1.5 MS: [M + H] = 392.7 | 102 | 28 |
| 80 | N-[3-(5,6- dimethyl-1H- benzimidazol-2- yl)cyclohexyl]- 2,3-dihydro-1,4- benzodioxine- 6-carboxamide HCl | 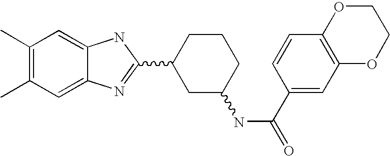 | HPLC Rt = 1.8 MS: [M + H] = 406.1 | 103 | 28 |
| 81 | N-[3-(6,7- difluoro-1H- benzimidazol-2- yl)cyclohexyl]- 2,3-dihydro-1,4- benzodioxine- 6-carboxamide HCl | 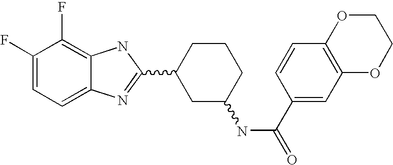 | HPLC Rt = 2.1 MS: [M + H] = 414 | 103 | 28 |
| 82 | N-[3-(6-bromo- 4,5-dimethyl- 1H- benzimidazol-2- yl)cyclohexyl]- 2,3-dihydro-1,4- benzodioxine- 6-carboxamide HCl | 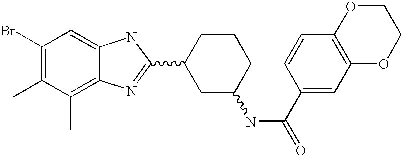 | HPLC Rt = 2.1 MS: [M + H] = 484 | 101 | 28 |
| 83 | N-[3-(5,6- difluoro-1H- benzimidazol-2- yl)cyclohexyl]- 2,3-dihydro-1,4- benzodioxine- 6-carboxamide HCl | 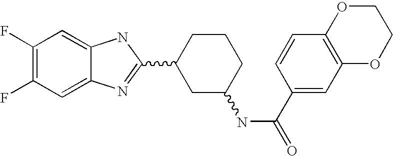 | HPLC Rt = 1.8 MS: [M + H] = 414 | 103 | 28 |
| 84 | N-{3-[6- (trifluoromethyl)- 1H- benzimidazol-2- yl]cyclohexyl}- 2,3-dihydro-1,4- benzodioxine- 6-carboxamide- HCl | 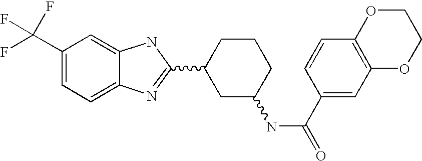 | HPLC Rt = 2.2 MS: [M + H] = 446 | 101 | 28 |
| 85 | N-[3-(6-chloro- 1H- benzimidazol-2- yl)cyclohexyl]- 2,3-dihydro-1,4- benzodioxine- 6-carboxamide HCl | 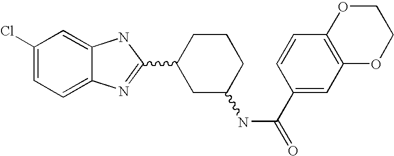 | HPLC Rt = 1.8 MS: [M + H] = 412 | 103 | 28 |
| 86 | N-[3-(6-bromo- 1H- benzimidazol-2- yl)cyclohexyl]- 2,3-dihydro-1,4- benzodioxine- 6-carboxamide HCl | 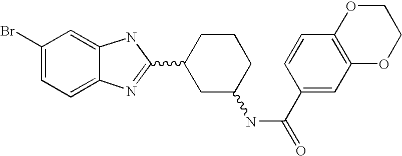 | HPLC Rt = 1.8 MS: [M + H] = 456 | 102 | 28 |
| 87 | N-[5-(1H- benzimidazol-2- yl)bicyclo[3.1.1] hept-1-yl]-2,3- dihydro-1,4- benzodioxine- 6-carboxamide | 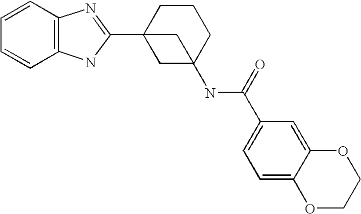 | HPLC Rt = 1.5 MS: [M + H] = 390.1 | 91 | 51 |
| 88 | N-[(1R,3S)-3- (5-methoxy-1H- benzimidazol-2- yl)cyclohexyl]- 2,3-dihydro-1,4- benzodioxine- 6-carboxamide HCl | 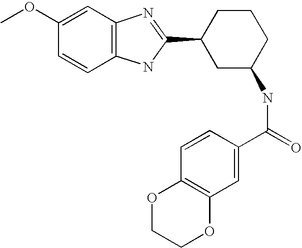 | HPLC Rt = 1.4 MS: [M + H] = 408.1 | 106 | 28 |
| 89 | N-[3-(1H- benzimidazol-2- yl)cyclohexyl]- 4-oxo-4- phenylbutanamide triflouroacetate | 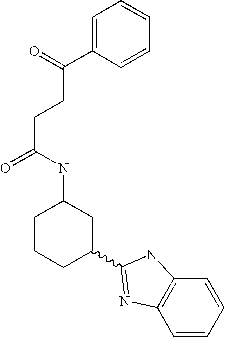 | HPLC Rt = 2.47 MS: [M + H] = 376.26 | 96 | 46 |
| 90 | N-[3-(1H- benzimidazol-2- yl)cyclohexyl]- 4-(1H-imidazol- 1-yl)benzamide triflouroacetate | 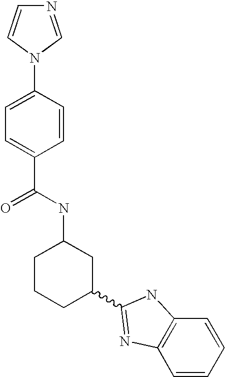 | HPLC Rt = 1.9 MS: [M + H] = 386.3 | 100 | 46 |
| 91 | N-[3-(1H- benzimidazol-2- yl)cyclohexyl]- 1-benzyl-1H- pyrazole-4- carboxamide triflouroacetate | 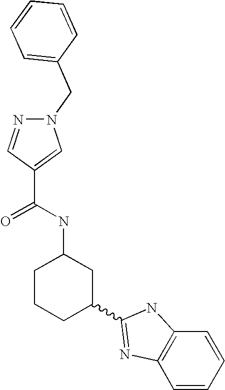 | HPLC Rt = 2.53 MS: [M + H] = 400.27 | 99 | 46 |
| 92 | N-[3-(1H- benzimidazol-2- yl)cyclohexyl]- 2-(2H-1,2,3- benzotriazol-2- yl)acetamide triflouroacetate | 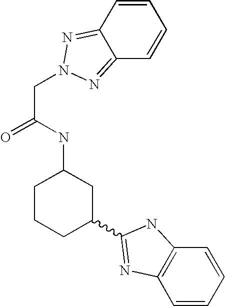 | HPLC Rt = 2.38 MS: [M + H] = 375.26 | 90 | 46 |
| 93 | N-[3-(1H- benzimidazol-2- yl)cyclohexyl]- 3-methyl-2- furamide triflouroacetate | 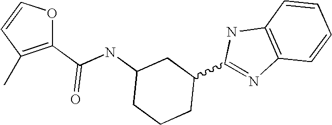 | HPLC Rt = 2.32 MS: [M + H] = 324.29 | 24 | 46 |
| 94 | N-[3-(1H- benzimidazol-2- yl)cyclohexyl]- 2-(4-methyl- 3,4-dihydro-2H- 1,4- benzoxazin-3- yl)acetamide triflouroacetate | 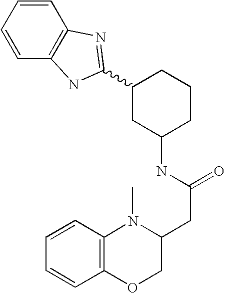 | HPLC Rt = 2.68 MS: [M + H] = 405.27 | 96 | 46 |
| 95 | N-[3-(1H- benzimidazol-2- yl)cyclohexyl]- 2-(1H-indazol- 1-yl)acetamide triflouroacetate | 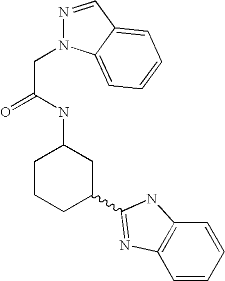 | HPLC Rt = 2.42 MS: [M + H] = 374.25 | 20 | 46 |
| 96 | N-[3-(1H- benzimidazol-2- yl)cyclohexyl]- 4- (methoxymeth- oxy)benzamide triflouroacetate | 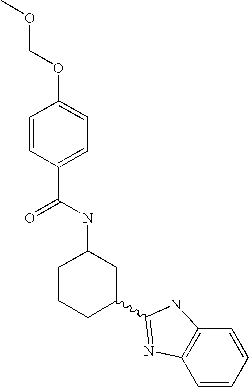 | HPLC Rt = 2.47 MS: [M + H] = 380.28 | 93 | 46 |
| 97 | N-[3-(1H- benzimidazol-2- yl)cyclohexyl]- 3-isopropyl-4,5- dihydroisoxazole- 5- carboxamide triflouroacetate | 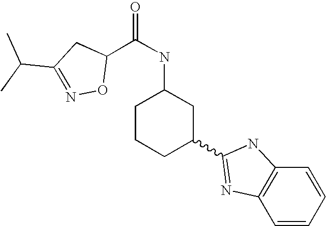 | HPLC Rt = 2.27 MS: [M + H] = 355.3 | 83 | 46 |
| 98 | ethyl 6-{[3-(1H- benzimidazol-2- yl)cyclohexyl] amino}-6- oxohexanoate trifl;uoroacetate | 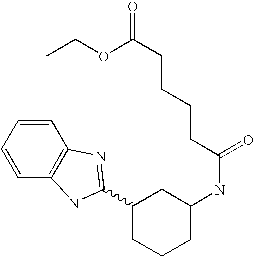 | HPLC Rt = 2.36 MS: [M + H] = 372.3 | 55 | 46 |
| 99 | 4-acetyl-N-[3- (1H- benzimidazol-2- yl)cyclohexyl]- 3,5-dimethyl- 1H-pyrrole-2- carboxamide triflouroacetate | 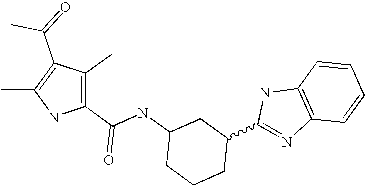 | HPLC Rt = 2.3 MS: [M + H] = 379.28 | 107 | 46 |
| 100 | N-[5-(1H- benzimidazol-2- yl)bicyclo[3.1.1] hept-1-yl]-3,5- dimethoxybenz- amide | 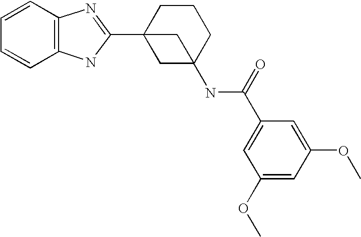 | HPLC Rt = 1.6 MS: [M + H] = 392.1 | 81 | 51 |
| 101 | N-[3-(1H- benzimidazol-2- yl)cyclohexyl]- 1H-indazole-3- carboxamide triflouroacetate | 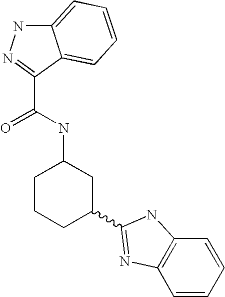 | HPLC Rt = 0.54 MS: [M + H] = 360.16 | 56 | 46 |
| 102 | N-[3-(1H- benzimidazaol-2- yl)cyclohexyl]- 2- cyanoacetamide triflouroacetate | 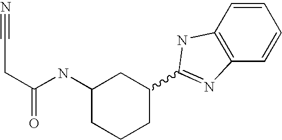 | HPLC Rt = 0.44 MS: [M + H] = 283.17 | 93 | 46 |
| 103 | methyl 5-{[3- (1H- benzimidazol-2- yl)cyclohexyl]- amino}-5- oxopentanoate triflouroacetate | 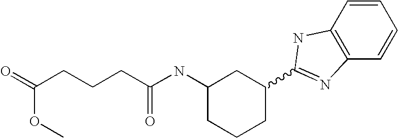 | HPLC Rt = 0.48 MS: [M + H] = 344.19 | 94 | 46 |
| 104 | N-[3-(1H- benzimidazol-2- yl)cyclohexyl]- 2- (benzyloxy)- acetamide triflouroacetate | 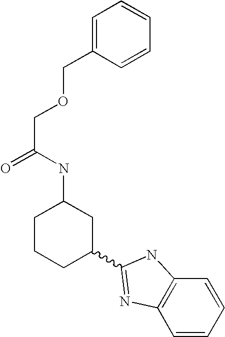 | HPLC Rt = 0.58 MS: [M + H] = 364.2 | 73 | 46 |
| 105 | N-[3-(1H- benzimidazol-2- yl)cyclohexyl]- 2-(3-oxo-2,3- dihydro-4H- pyrido[3,2- b][1,4]oxazin-4- yl)acetamide triflouroacetate | 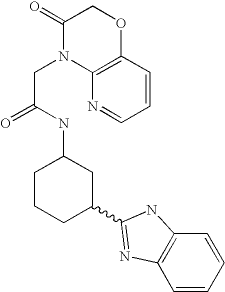 | HPLC Rt = 0.5 MS: [M + H] = 406.18 | 103 | 46 |
| 106 | N-[3-(1H- benzimidazol-2- yl)cyclohexyl]- 1-phenyl-1H- pyrazole-5- carboxamide triflouroacetate | 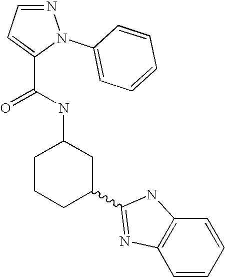 | HPLC Rt = 0.53 MS: [M + H] = 386.2 | 76 | 46 |
| 107 | N-[3-(1H- benzimidazol-2- yl)cyclohexyl]- 2-(6- chloroimidazo[1,2- a]pyridin-2- yl)acetamide triflouroacetate | 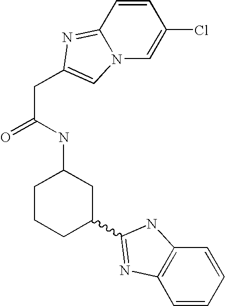 | HPLC Rt = 0.45 MS: [M + H] = 408.14 | 79 | 46 |
| 108 | N-[3-(1H- benzimidazol-2- yl)cyclohexyl]- 2,4-dioxo- 1,2,3,4- tetrahydropyr- imidine-5- carboxamide triflouroacetate | 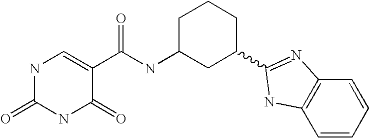 | HPLC Rt = 0.44 MS: [M + H] = 354.15 | 116 | 46 |
| 109 | N-[3-(1H- benzimidazol-2- yl)cyclohexyl]- 2- methoxynico- tinamide triflouroacetate | 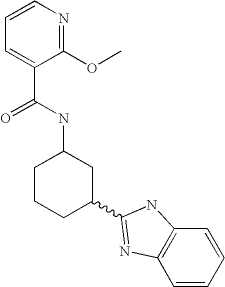 | HPLC Rt = 0.52 MS: [M + H] = 351.16 | 97 | 46 |
| 110 | N-[3-(1H- benzimidazol-2- yl)cyclohexyl]- 2-(3-oxo-1- propylpiperazin- 2-yl)acetamide triflouroacetate | 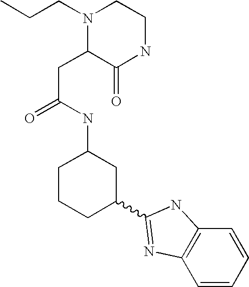 | HPLC Rt = 0.42 MS: [M + H] = 398.24 | 86 | 46 |
| 111 | N-[3-(1H- benzimidazol-2- yl)cyclohexyl]- 2-(6-methyl-2- oxopyridin- 1(2H)- yl)acetamide triflouroacetate | 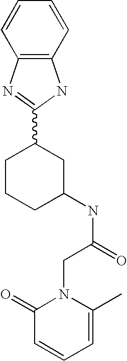 | HPLC Rt = 0.46 MS: [M + H] = 365.17 | 103 | 46 |
| 112 | N-[3-(1H- benzimidazol-2- yl)cyclohexyl]- 2-(2- oxoazepan-1- yl)acetamide triflouroacetate | 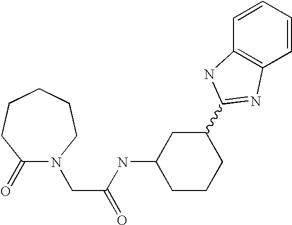 | HPLC Rt = 0.48 MS: [M + H] = 369.22 | 93 | 46 |
| 113 | (4R)-N-[3-(1H- benzimidazol-2- yl)cyclohexyl]- 4-hydroxy-1- methyl-L- prolinamide triflouroacetate | 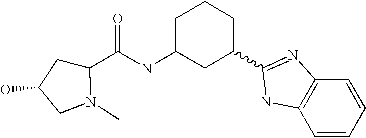 | HPLC Rt = 0.41 MS: [M + H] = 343.21 | 93 | 46 |
| 114 | N-[3-(1H- benzimidazol-2- yl)cyclohexyl]- 3-cyclohexyl]- 1H-pyrazole-5- carboxamide triflouroacetate | 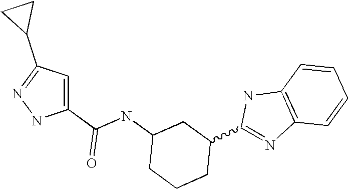 | HPLC Rt = 0.5 MS: [M + H] = 350.18 | 109 | 46 |
| 115 | N-[3-(1H- benzimidazol-2- yl)cyclohexyl]- 2-(1-methyl-1H- indol-3- yl)acetamide triflouroacetate | 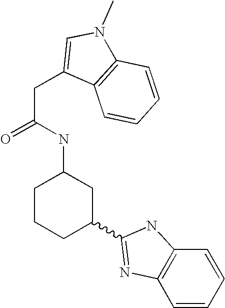 | HPLC Rt = 0.59 MS: [M + H] = 387.21 | 106 | 46 |
| 116 | N-[3-(1H- benzimidazol-2- yl)cyclohexyl]- 3-(2- methylimidazo[2,1- b][1,3]thiazol-6- yl)propanamide triflouroacetate | 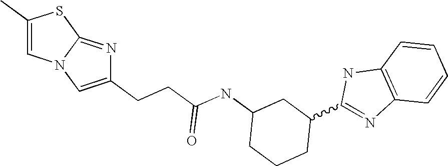 | HPLC Rt = 0.44 MS: [M + H] = 408.2 | 91 | 46 |
| 117 | N-(2-{[3-(1H- benzimidazol-2- yl)cyclohexyl] amino}-2- oxoethyl)benz- amide triflouroacetate | 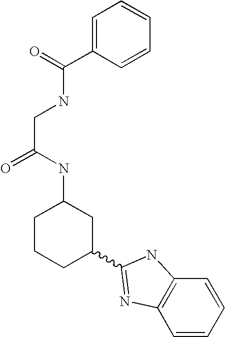 | HPLC Rt = 0.5 MS: [M + H] = 377.18 | 102 | 46 |
| 118 | N-[3-(1H- benzimidazol-2- yl)cyclohexyl]- 6-methoxy-1- methyl-1H- indole-2- carboxamide triflouroacetate | 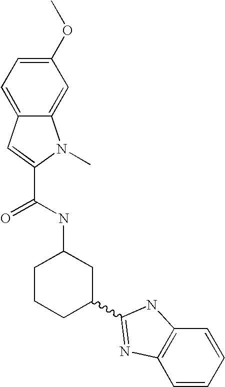 | HPLC Rt = 0.61 MS: [M + H] = 403.19 | 83 | 46 |
| 119 | N-[3-(1H- benzimidazol-2- yl)cyclohexyl]- 2,6- dimethoxypyr- imidine-4- carboxamide triflouroacetate | 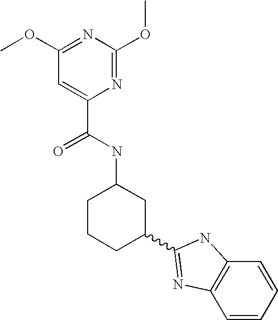 | HPLC Rt = 0.57 MS: [M + H] = 382.19 | 75 | 46 |
| 120 | N-[3-(1H- benzimidazol-2- yl)cyclohexyl]- 3-(3,5-dimethyl- 1H-pyrazol-1- yl)propanamide triflouroacetate | 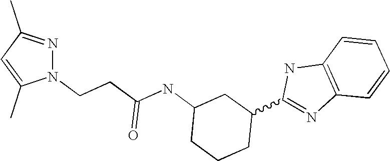 | HPLC Rt = 0.46 MS: [M + H] = 366.22 | 70 | 46 |
| 121 | N-[3-(1H- benzimidazol-2- yl)cyclohexyl] tetrahydrofuran-2- carboxamide triflouroacetate | 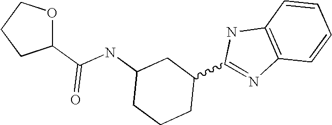 | HPLC Rt = 0.46 MS: [M + H] = 314.2 | 94 | 46 |
| 122 | N-(3-(1H- benzo[d]imida- zol-2- yl)cyclohexyl)- 4,5,6,7- tetrahydropyr- azolo[1,5- a]pyridine-3- carboxamide triflouroacetate | 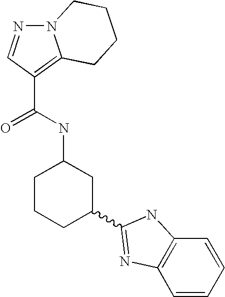 | HPLC Rt = 0.49 MS: [M + H] = 364.2 | 92 | 46 |
| 123 | Nalpha-acetyl- N-[3-(1H- benzimidazol-2- yl)cyclohexyl]- D- phenylalanin- amide triflouroacetate | 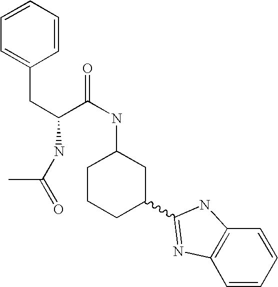 | HPLC Rt = 0.52 MS: [M + H] = 405.2 | 83 | 46 |
| 124 | N-[3-(1H- benzimidazol-2- yl)cyclohexyl]- 2-chloro-6- methylnico- tinamide triflouroacetate | 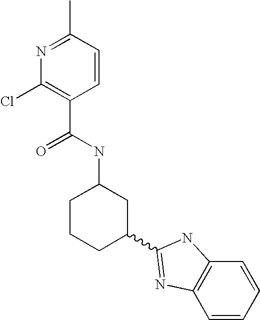 | HPLC Rt = 0.48 MS: [M + H] = 369.15 | 62 | 46 |
| 125 | N-[3-(1H- benzimidazol-2- yl)cyclohexyl]- 2-[(1S,2S,4S)- bicyclo[2.2.1]hept- 5-en-2- yl]acetamide triflouroacetate | 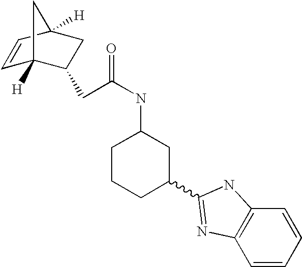 | HPLC Rt = 0.58 MS: [M + H] = 350.22 | 103 | 46 |
| 126 | N-[3-(1H- benzimidazol-2- yl)cyclophexyl]- 4- (methylsulfinyl) benzamide triflouroacetate | 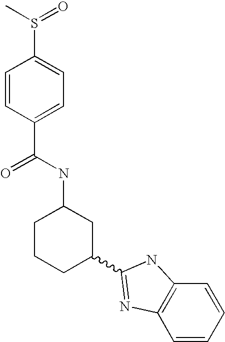 | HPLC Rt = 0.45 MS: [M + H] = 382.12 | 83 | 46 |
| 127 | N-[3-(1H- benzimidazol-2- yl)cyclohexyl]- 2-(4- oxothieno[3,2- c]pyridin-5(4H)- yl)acetamide HCl | 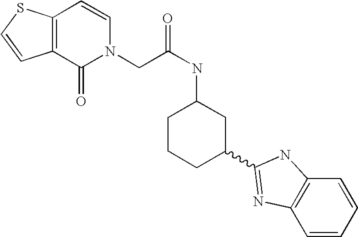 | HPLC Rt = 0.5 MS: [M + H] = 407.15 | 100 | 46 |
| 128 | N-[3-(1H- benzimidazol-2- yl)cyclohexyl]- 2-(4- oxothieno[3,2- c]pyridin-5(4H)- yl)acetamide triflouroacetate | 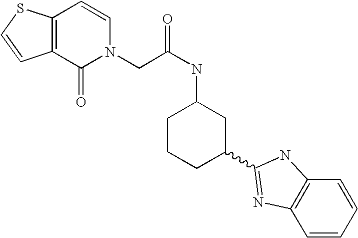 | HPLC Rt = 0.5 MS: [M + H] = 407.15 | 100 | 46 |
| 129 | N-[3-(1H- benzimidazol-2- yl)cyclohexyl]- N′- phenylpentane diamide triflouroacetate | 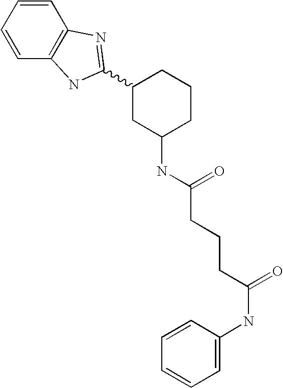 | HPLC Rt = 0.53 MS: [M + H] = 405.2 | 95 | 46 |
| 130 | N-[3-(1H- benzimidazol-2- yl)cyclohexyl]- 1-methyl-4- (pyrrolidin-1- ylmethyl)-1H- pyrrole-2- carboxamide triflouroacetate | 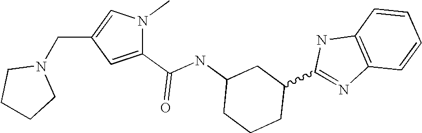 | HPLC Rt = 0.44 MS: [M + H] = 406.25 | 84 | 46 |
| 131 | N-[3-(1H- benzimidazol-2- yl)cyclohexyl]- 2-(5-chloro-2- thienyl)acetamide triflouroacetate | 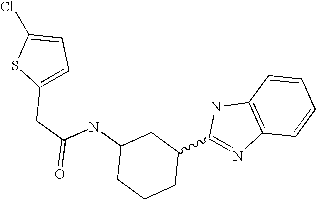 | H_PLC Rt = 0.59 MS: [M + H] = 374.09 | 101 | 46 |
| 132 | N-[3-(1H- benzimidazol-2- yl)cyclohexyl]- 1-(4- methoxyphenyl) cyclopropane- carboxamide triflouroacetate | 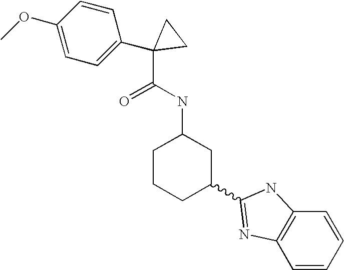 | HPLC Rt = 0.61 MS: [M + H] = 390.2 | 78 | 46 |
| 133 | N-[3-(1H- benzimidazol-2- yl)cyclohexyl]- 8- methoxychrom- ane-3- carboxamide triflouroacetate | 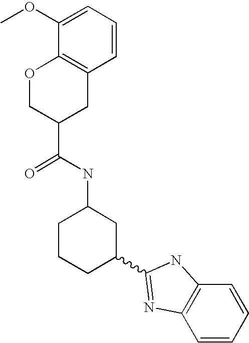 | HPLC Rt = 0.56 MS: [M + H] = 406.18 | 85 | 46 |
| 134 | N-[3-(1H- benzimidazol-2- yl)cyclohexyl]- 5-(morpholin-4- ylmethyl)-2- furamide triflouroacetate | 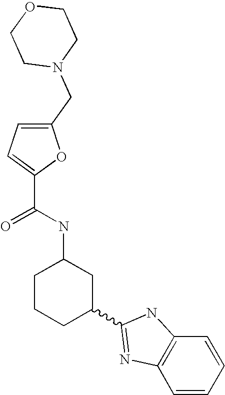 | HPLC Rt = 0.43 MS: [M + H] = 409.18 | 101 | 46 |
| 135 | N-[3-(1H- benzimidazol-2- yl)cyclohexyl]- 3-pyridin-4-yl- 1H-pyrazole-5- carboxamide triflouroacetate | 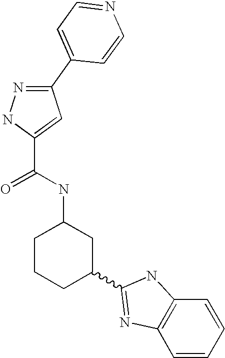 | HPLC Rt = 0.43 MS: [M + H] = 387.16 | 100 | 46 |
| 136 | (2E)-N-[3-(1H- benzimidazol-2- yl)cyclohexyl]- 3-(3,5-dimethyl- 1H-pyrazol-4- yl)acrylamide triflouroacetate | 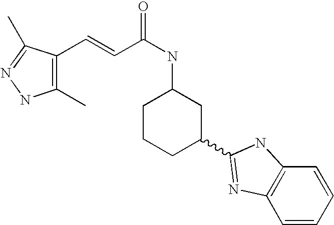 | HPLC Rt = 0.45 MS: [M + H] = 364.2 | 86 | 46 |
| 137 | N-[3-(1H- benzimidazol-2- yl)cyclohexyl]- 2-oxo-1,2,3,4- tetrahydroquino- line-6- carboxamide triflouroacetate | 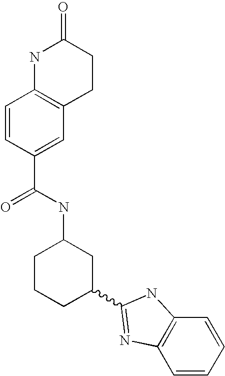 | HPLC Rt = 0.47 MS: [M + H] = 389.16 | 108 | 46 |
| 138 | 2-[(1S,4R)-2- azabicyclo[2.2.1] hept-2-yl]-N- [3-(1H- benzimidazol-2- yl)cyclohexyl]- acetamide triflouroacetate | 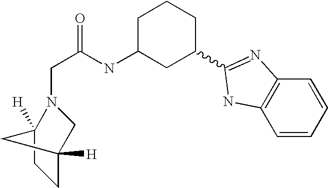 | HPLC Rt = 0.42 MS: [M + H] = 353.19 | 100 | 46 |
| 139 | N-[3-(1H- benzimidazol-2- yl)cyclohexyl]- 8-methoxy-2,3- dihydro-1,4- benzodioxine- 6-carboxamide triflouroacetate | 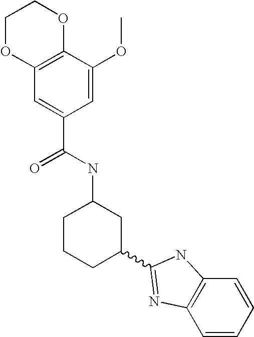 | HPLC Rt = 0.56 MS: [M + H] = 408.21 | 105 | 46 |
| 140 | N-[3-(1H- benzimidazol-2- yl)cyclohexyl]- 1H-indole-6- carboxamide HCl | 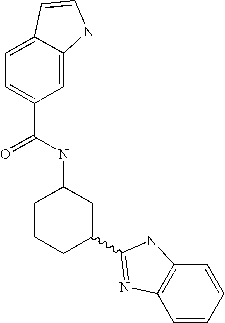 | HPLC Rt = 0.62 MS: [M + H] = 359.18 | 110 | 46 |
| 141 | N-[3-(1H- benzimidazol-2- yl)cyclohexyl]- 1H-indole-6- carboxamide triflouroacetate | 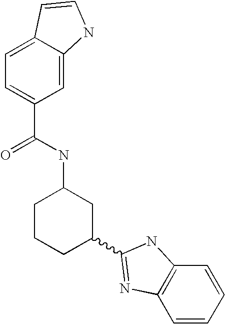 | HPLC Rt = 0.62 MS: [M + H] = 359.18 | 110 | 46 |
| 142 | N-[3-(1H- benzimidazol-2- yl)cyclohexyl]- 2-(1H-pyrazol- 1- yl)propanamide triflouroacetate | 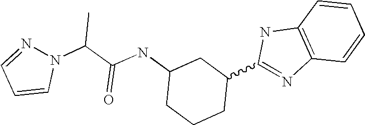 | HPLC Rt = 0.49 MS: [M + H] = 338.27 | 69 | 46 |
| 143 | N-[3-(1H- benzimidazol-2- yl)cyclohexyl]- 1-methyl-2-oxo- 2,3-dihydro-1H- benzimidazole- 5-carboxamide triflouroacetate | 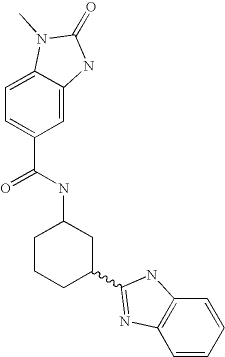 | HPLC Rt = 0.5 MS: [M + H] = 390.21 | 87 | 46 |
| 144 | N-[3-(1H- benzimidazol-2- yl)cyclohexyl]- 2-(5-chloro-1H- benzimidazol-2- yl)acetamide HCl | 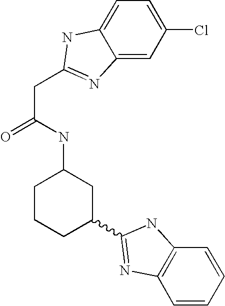 | HPLC Rt = 0.52 MS: [M + H] = 408.17 | 105 | 46 |
| 145 | N-[3-(1H- benzimidazol-2- yl)cyclohexyl]- 2-(5-chloro-1H- benzimidazol-2- yl)acetamide triflouroacetate | 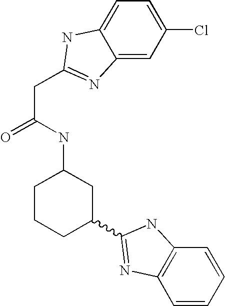 | HPLC Rt = 0.52 MS: [M + H] = 408.17 | 105 | 46 |
| 146 | N-[3-(1H- benzimidazol-2- yl)cyclohexyl]- 1-ethyl-3- isopropyl-1H- pyrazole-5- carboxamide triflouroacetate | 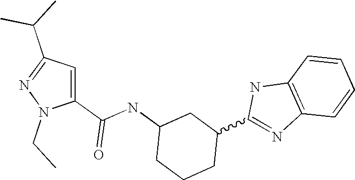 | HPLC Rt = 0.66 MS: [M + H] = 380.26 | 61 | 46 |
| 147 | N-[3-(1H- benzimidazol-2- yl)cyclohexyl]- 3-chloro-4- hydroxybenz- amide triflouroacetate | 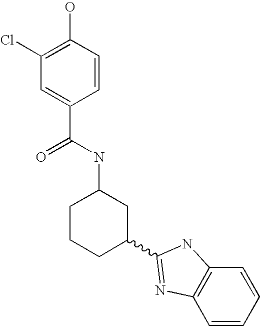 | HPLC Rt = 0.56 MS: [M + H] = 370.13 | 68 | 46 |
| 148 | 3-(1H- benzimidazol-2- yl)-N-[3-(1H- benzimidazol-2- yl)cyclohexyl]- propanamide triflouroacetate | 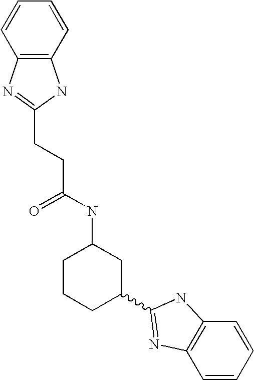 | HPLC Rt = 0.45 MS: [M + H] = 388.21 | 50 | 46 |
| 149 | N-[(1R,3S)-3- (5-bromo-1H-benzimidazol-2- yl)cyclohexyl]- 2,3-dihydro-1,4- benzodioxine- 6-carboxamide HCl | 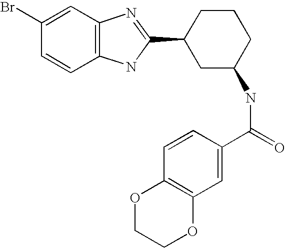 | HPLC Rt = 1.8 MS: [M + H] = 456 | 103 | 28 |
| 150 | N-[(1S,3R)-3- (5-bromo-1H- benzimidazol-2- yl)cyclohexyl]- 2,3-dihydro-1,4- benzodioxine- 6-carboxamide HCl | 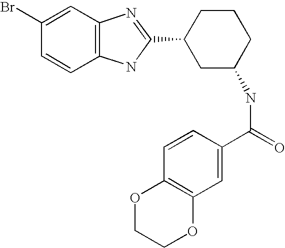 | HPLC Rt = 1.8 MS: [M + H] = 456 | 45 | 28 |
| 151 | N-{(1R,3S)-3- [5- (trifluoromethyl)- 1H- benzimidazol-2- yl]cyclohexyl}- 2,3-dihydro-1,4- benzodioxine- 6-carboxamide HCl | 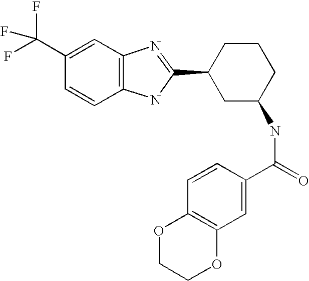 | HPLC Rt = 2.9 MS: [M + H] = 446.1 | 106 | 28 |
| 152 | N-{(1S,3R)-3- [5- (trifluoromethyl)- 1H- benzimidazol-2- yl]cyclohexyl}- 2,3-dihydro-1,4- benzodioxine- 6-carboxamide HCl | 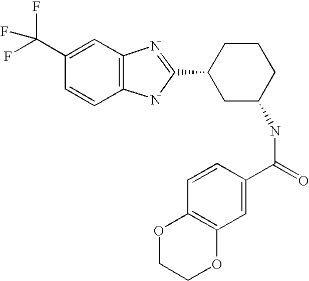 | HPLC Rt = 2.9 MS: [M + H] = 446.1 | 80 | 28 |
| 153 | N-[3-(1H- benzimidazol-2- yl)cyclohexyl]- 3-pyridin-2-yl- 1H-pyrazole-5- carboxamide triflouroacetate | 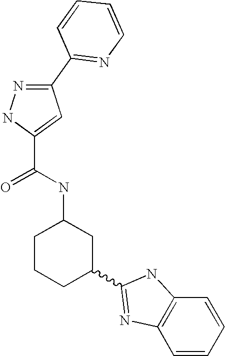 | HPLC Rt = 1.98 MS: [M + H] = 387.29 | 46 | 46 |
| 154 | N-[3-(1H- benzimidazol-2- yl)cyclohexyl]- 2-(1- oxoisoquinolin- 2(1H)- yl)acetamide triflouroacetate | 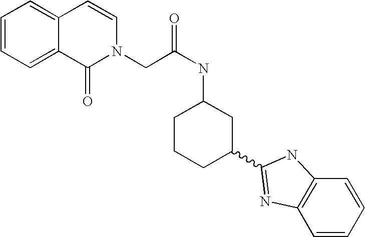 | HPLC Rt = 2.38 MS: [M + H] = 401.25 | 57 | 46 |
| 155 | N-[3-(1H- benzimidazol-2- yl)cyclohexyl]- 3-ethyl-4- methyl-1H- pyrazol-5- carboxamide triflouroacetate | 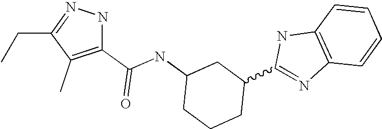 | HPLC Rt = 2.38 MS: [M + H] = 352.32 | 64 | 46 |
| 156 | N-[3-(1H- benzimidazol-2- yl)cyclohexyl]- 3-(7- methylimidazo [1,2-a]pyridin-2- yl)propanamide triflouroacetate | 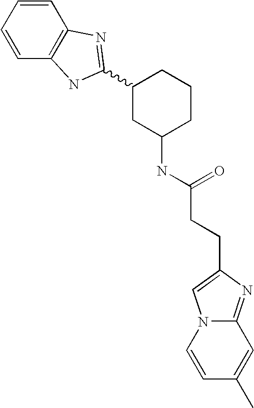 | HPLC Rt = 1.96 MS: [M + H] = 402.34 | 53 | 46 |
| 157 | 2-(4- acetylphenoxy)- N-[3-(1H- benzimidazol-2- yl)cyclohexyl] acetamide triflouroacetate | 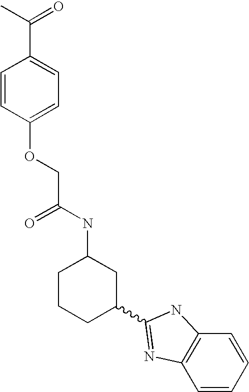 | HPLC Rt = 2.39 MS: [M + H] = 392.28 | 52 | 46 |
| 158 | N-[3-(1H- benzimidazol-2- yl)cyclohexyl]- 2,4-dioxo- 1,2,3,4- tetrahydroquin- azoline-6- carboxamide triflouroacetate | 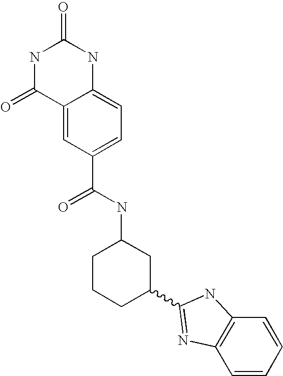 | HPLC Rt = 2.05 MS: [M + H] = 404.26 | 73 | 46 |
| 159 | N-[3-(1H- benzimidazol-2- yl)cyclohexyl]- 4-(1-methyl-1H- imidazol-2- yl)benzamide triflouroacetate | 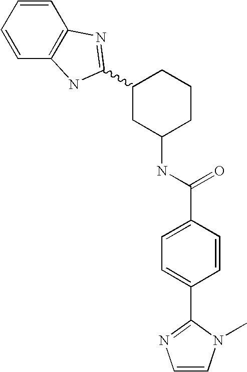 | HPLC Rt = 1.91 MS: [M + H] = 400.28 | 78 | 46 |
| 160 | N-[3-(1H- benzimidazol-2- yl)cyclohexyl]- 4-(1,3-oxazol-5- yl)benzamide triflouroacetate | 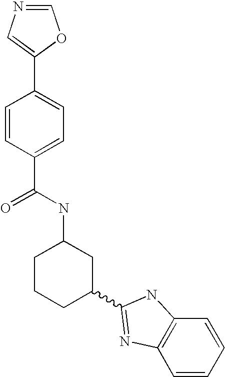 | HPLC Rt = 2.37 MS: [M + H] = 387.24 | 72 | 46 |
| 161 | N-[3-(1H- benzimidazol-2- yl)cyclohexyl]- 1,2,3- benzothiadiazole- 5- carboxamide triflouroacetate | 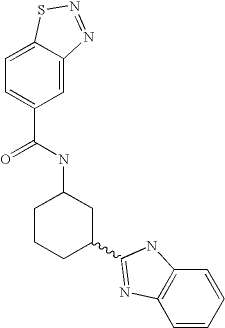 | HPLC Rt = 2.43 MS: [M + H] = 378.21 | 54 | 46 |
| 162 | N-[3-(1H- benzimidazol-2- yl)cyclohexyl]- 2- {(dimethylamino)- sulfonyl]- acetamide triflouroacetate | 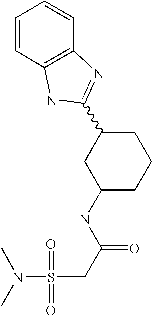 | HPLC Rt = 2.04 MS: [M + H] = 365.23 | 71 | 46 |
| 163 | N-[3-(1H- benzimidazol-2- yl)cyclohexyl]- 4-(1H-tetrazol- 1-yl)benzamide triflouroacetate | 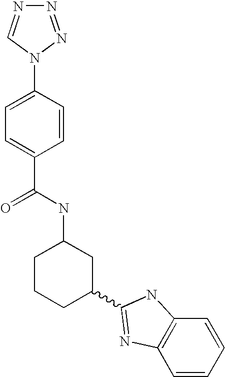 | HPLC Rt = 2.26 MS: [M + H] = 388.27 | 95 | 46 |
| 164 | N-[3-(1H- benzimidazol-2- yl)cyclohexyl]- 1-methyl-1H- imidazole-2- carboxamide triflouroacetate | 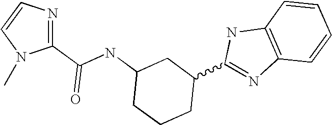 | HPLC Rt = 1.84 MS: [M + H] = 324.38 | 106 | 46 |
| 165 | N-[(1R,3S)-3- (1H- benzimidazol-2- yl)cyclohexyl]- 2- methoxyisonico- tinamide HCl | 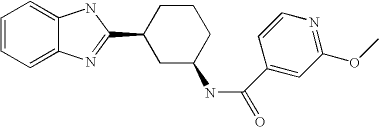 | HPLC Rt = 1.8 MS: [M + H] = 351 | 63 | 5 |
| 166 | N-[(1R,3S)-3- (1H- benzimidazol-2- yl)cyclohexyl]- 3,5- dimethoxybenz- amide HCl | 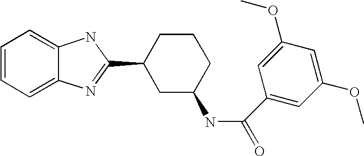 | 1H NMR (methanol-d₄, 400 MHz) 1.5-1.6 (m, 1H), 1.65-1.8 (m, 3H), 2.0- 2.1 (m, 2H), 2.1-2.15 (m, 1H), 2.45- 2.5 (m, 1H), 3.35-3.45 (m, 1H), 3.75 (s, 6H), 4.0-4.1 (m, 1H), 6.6 (s,1H), 6.95 (s, 2H), 7.5- 7.55 (m, 2H), 7.7-7.75 (m, 2H); HPLC Rt = 1.5 MS: [M + H] = 380 | 109 | 5 |
| 167 | 3,5-dimethoxy- N[(1R,3S)-3- (1-methyl-1H- benzimidazol-2- yl)cyclohexyl] benzamide HCl | 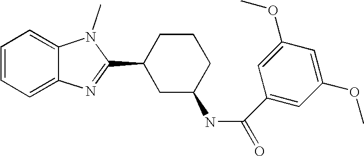 | 1H NMR (methanol- d4, 400 MHz) 1.5-1.6 (m, 1H), 1.65-1.8 (m, 3H), 2.0-2.1 (m, 2H), 2.1- 2.15 (m, 1H), 2.4-2.45 (m, 1H), 3.55-3.6 (m, 1H), 3.75 (s, 6H), 4.05 (s, 3H), 4.1- 4.15 (m, 1H), 6.6 (s, 1H), 6.95 (s, 2H), 7.75-7.8 (m, 2H), 7.75 (d, 1H), 7.85 (d, 1H); HPLC Rt = 2 MS: [M + H] = 394.1 | 101 | 3 |
| 168 | N-[(1R,3S)-3- (6-methyl-1H- benzimidazol-2- yl)cyclohexyl]- 2,3-dihydro-1,4- benzodioxine- 6-carboxamide HCl | 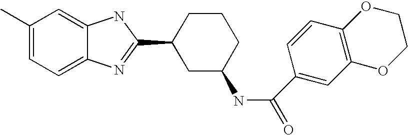 | 1H NMR (methanol-d₄, 400 MHz) 1.4-1.5 (m, 1H), 1.55- 1.65 (m, 2H), 1.65-1.8 (m, 1H), 1.9- 2.05 (m, 3H), 2.25-2.3 (m, 1H), 2.4 (s, 3H), 2.95-3.05 (m, 1H), 3.95- 4.05 (m, 1H), 4.2 (s, 4H), 6.8 (d, 1H), 7.0 (d, 1H), 7.25 (s, 1H), 7.3-7.35 (m, 3H); HPLC Rt =1.8 MS: [M + H] = 392.1 | 106 | 4 |
| 169 | N-[(1R,3S)-3- (1H- benzimidazol-2- yl)cyclohexyl]- 5-chloro-6- hydroxynicotin- amide HCl | 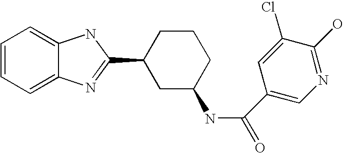 | HPLC Rt = 1.6 MS: [M + H] = 371.2 | 93 | 5 |
| 170 | 3,5-dimethoxy- N-{(1R,3S)-3- [6- (trifluoromethyl)- 1H- benzimidazol-2- yl]cyclohexyl} benzamide HCl | 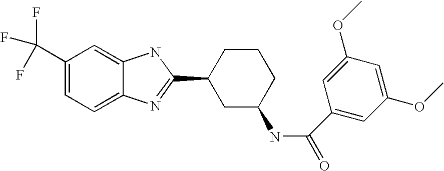 | 1H NMR (methanol-d₄, 400 MHz) 1.4-1.5 (m, 1H),1.6-1.65 (m, 2H), 1.7- 1.8 (m, 1H), 1.95-2.05 (m, 2H), 2.1- 2.15 (m, 1H), 2.3-2.35 (m, 1H), 3.05-3.15 (m, 1H), 3.75 (s, 6H), 4.0-4.1 (m, 1H), 6.6 (s, 1H), 6.95 (s, 2H), 7.45 (d, 1H), 7.6 (d, 1H), 7.75 (s, 1H); HPLC Rt = 2.9 MS: [M+ H] = 448.4 | 110 | 4 |
| 171 | 3,5-dimethoxy- N-{(1R,3S)-3- [6- (trifluoromethyl)- 1H- benzimidazol-2- yl]cyclohexyl} benzamide (+)- tartrate | 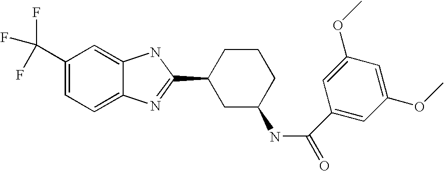 | HPLC Rt = 2.9 MS: [M + H] = 448.4 | 107.2 | 4 |
| 172 | N-[(1R,3S)-3- (6-cyano-1H- benzimidazol-2- yl)cyclohexyl]- 2,3-dihydro-1,4- benzodioxine- 6-carboxamide HCl | 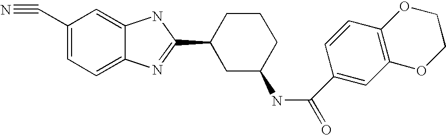 | 1H NMR (methanol- d4, 400 MHz) 1.4-1.5 (m, 1H), 1.55-1.65 (m, 2H), 1.65- 1.75 (m, 1H), 1.9-2.0 (m, 2H), 2.05-2.15 (m, 1H), 2.3- 2.35 (m, 1H), 3.05-3.15 (m, 1H), 3.95-4.05 (m, 1H), 4.2 (s, 4H), 6.8 (d, 1H), 7.25- 7.35 (m, 2H), 7.5 (d, 1H), 7.55-7.6 (m, 1H), 7.85-7.9 (m, 1H); HPLC Rt= 2.6 MS: [M + H] = 403.4 | 90 | 28 |
| 173 | N-[(1R,3S)-3- (1H- benzimidazol-2- yl)cyclohexyl]- 3-oxo-3,4- dihydro-2H-1,4- benzoxazine-6- carboxamide HCl | 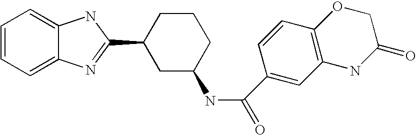 | HPLC Rt = 2 MS: [M + H] = 391.2 | 97 | 5 |
| 174 | N-[(1R,3S)-3- (1H- benzimidazol-2- yl)cyclohexyl]- 1-methyl-1H- imidazole-5- carboxamide HCl | 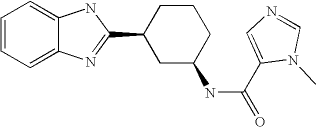 | HPLC Rt = 2.6 MS: [M + H] = 324.3 | 64 | 5 |
| 175 | N-[(1R,3S)-3- (1H- benzimidazol-2- yl)cyclohexyl]- 1-methyl-1H- benzimidazole- 2-carboxamide HCl | 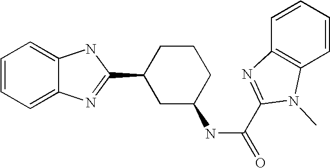 | HPLC Rt = 2.5 MS: [M + H] = 374.3 | 81 | 5 |
| 176 | N-[(1R,3S)-3- (1H- benzimidazol-2- yl)cyclohexyl]- 1H- benzimidazole- 2-carboxamide HCl | 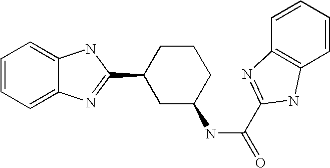 | 1H NMR (methanol-d₄, 400 MHz) 1.6-1.8 (m, 2H), 1.8-1.95 (m, 1H), 2.1- 2.3 (m, 3H), 2.55-2.65 (m, 1H), 3.4-3.5 (m, 1H), 3.75 (s, 6H), 4.15-4.25 (m, 1H), 7.5-7.6 (m, 4H), 7.75-7.8 (m, 2H), 7.8- 7.85 (m, 2H); HPLC Rt = 2.3 MS: [M + H] = 360.3 | 57 | 5 |
| 177 | N-[(1R,3S)-3- (1H- benzimidazol-2- yl)cyclohexyl]- 1,3-dimethyl- 1H-pyrazole-5- carboxamide HCl | 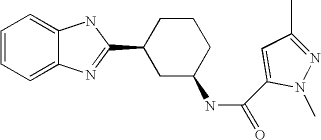 | HPLC Rt = 1.5 MS: [M + H] = 338.3 | 73 | 5 |
| 178 | N-[(1R,3S)-3- (6-chloro-1H- benzimidazol-2- yl)cyclohexyl]- 2,3-dihydro-1,4- benzodioxine- 6-carboxamide HCl | 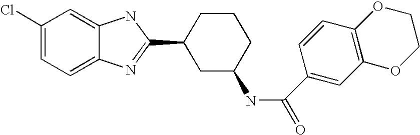 | HPLC Rt = 1.7 MS: [M + H] = 412 | 110 | 28 |
| 179 | N-[(1S,3R)-3- (6-chloro-1H- benzimidazol-2- yl)cyclohexyl]- 2,3-dihydro-1,4- benzodioxine- 6-carboxamide HCl | 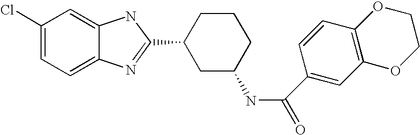 | HPLC Rt = 1.7 MS: [M + H] = 412 | 106 | 28 |
| 180 | N-[(1R,3S)-3- (6-methoxy-1H- benzimidazol-2- yl)cyclohexyl]- 2,3-dihydro-1,4- benzodioxine- 6-carboxamide HCl | 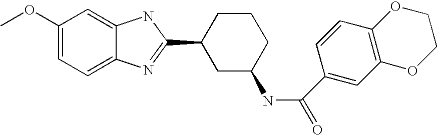 | 1H NMR (methanol-d₄, 400 MHz) 1.5-1.6 (m, 1H), 1.6-1.7 (m, 3H), 2.0- 2.1 (m, 2H), 2.15-2.25 (m, 1H), 2.4- 2.45 (m, 1H), 3.3-3.4 (m, 1H), 3.85 (s, 3H), 4.0-4.1 (m, 1H), 4.2 (s, 4H), 6.8 (d, 1H), 7.1-7.15 (m, 2H), 7.25-7.35 (m, 2H), 7.55 (d, 1H); HPLC Rt = 1.4 MS: [M + H] = 408.6 | 107 | 5 |
| 181 | N-[(1R,3S)-3- (1H- benzimidazol-2- yl)cyclohexyl]- 5-chloro-1- benzofuran-2- carboxamide triflouroacetate | 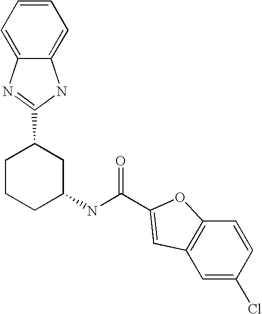 | HPLC Rt = 1.4 MS: [M + H] = 394.01 | 104 | 50 |
| 182 | N-[(1R,3S)-3- (1H- benzimidazol-2- yl)cyclohexyl]- 1- benzothiophene- 2- carboxamide triflouroacetate | 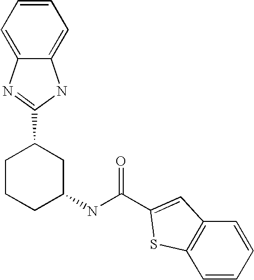 | HPLC Rt = 1.31 MS: [M + H] = 376.023 | 106 | 50 |
| 183 | N-[(1R,3S)-3- (1H- benzimidazol-2- yl)cyclohexyl]- 4-methyl-1- naphthamide triflouroacetate | 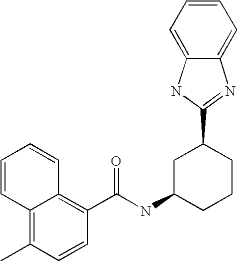 | HPLC Rt = 1.34 MS: [M + H] = 384.074 | 95 | 50 |
| 184 | N-[(1R,3S)-3- (1H- benzimidazol-2- yl)cyclohexyl]- 5-methoxy-1- benzofuran-2- carboxamide triflouroacetate | 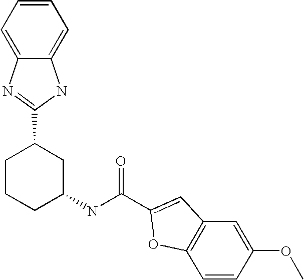 | HPLC Rt = 1.24 MS: [M + H] = 390.056 | 61 | 50 |
| 185 | (2S)-N- [(1R,3S)-3-(1H- benzimidazol-2- yl)cyclohexyl]- 2,3-dihydro-1,4- benzodioxine- 2-carboxamide triflouroacetate | 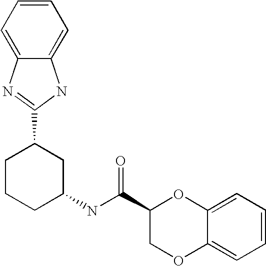 | HPLC Rt = 1.25 MS: [M + H] = 378.049 | 81 | 50 |
| 186 | N-[(1R,3S)-3- (1H- benzimidazol-2- yl)cyclohexyl]- 1,3- benzodioxole- 5-carboxamide triflouroacetate | 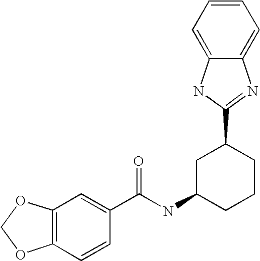 | HPLC Rt = 1.06 MS: [M + H] = 364.05 | 59 | 50 |
| 187 | N-[(1R,3S)-3- (1H- benzimidazol-2- yl)cyclohexyl]- 2-naphthamide triflouroacetate | 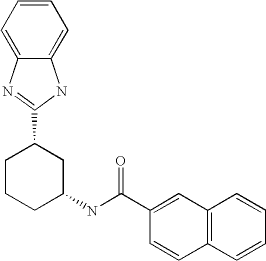 | HPLC Rt = 1.31 MS: [M + H] = 370.066 | 94 | 50 |
| 188 | N-[(1R,3S)-3- (1H- benzimidazol-2- yl)cyclohexyl]- 1-naphthamide triflouroacetate | 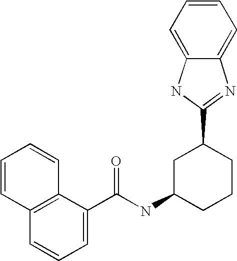 | HPLC Rt = 1.26 MS: [M + H] = 370.066 | 64 | 50 |
| 189 | N-[(1R,3S)-3- (1H- benzimidazol-2- yl)cyclohexyl]- 3-methyl-1- benzofuran-2- carboxamide triflouroacetate | 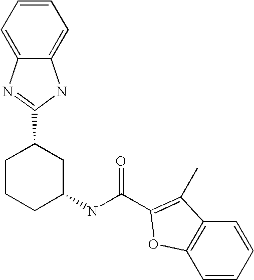 | HPLC Rt = 1.37 MS: [M + H] = 374.062 | 65 | 50 |
| 190 | N-[(1R,3S)-3- (1H- benzimidazol-2- yl)cyclohexyl]- 2,1,3- benzoxadiazole- 5-carboxamide triflouroacetate | 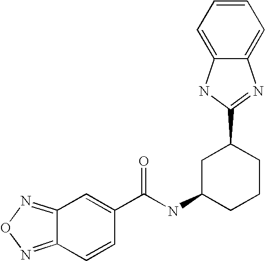 | HPLC Rt = 1.13 MS: [M + H] = 362.052 | 37 | 50 |
| 191 | N-[(1R,3S)-3- (1H- benzimidazol-2- yl)cyclohexyl]- 3,5-dimethyl- 1H-indole-2- carboxamide triflouroacetate | 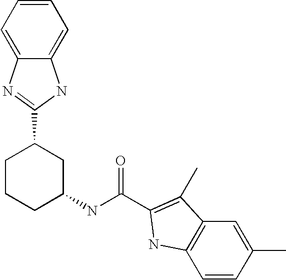 | HPLC Rt = 1.48 MS: [M + H] = 387.098 | 107 | 50 |
| 192 | N-[(1R,3S)-3- (1H- benzimidazol-2- yl)cyclohexyl]- 2,3-dimethyl- 1H-indole-5- carboxamide triflouroacetate | 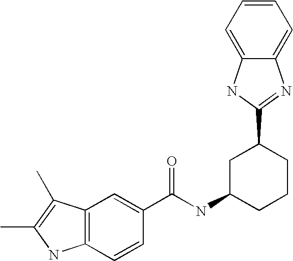 | HPLC Rt = 1.26 MS: [M + H] = 387.104 | 109 | 50 |
| 193 | N-[(1R,3S)-3- (1H- benzimidazol-2- yl)cyclohexyl]- 2H-chromene- 3-carboxamide triflouroacetate | 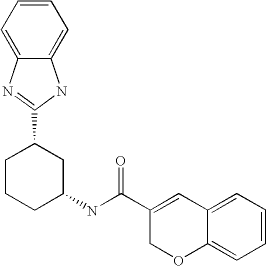 | HPLC Rt = 1.3 MS: [M + H] = 374.07 | 53 | 50 |
| 194 | N-[(1R,3S)-3- (1H- benzimidazol-2- yl)cyclohexyl]- 5,7- dimethylpyrazolo- [1,5- a]pyrimidine-2- carboxamide triflouroacetate | 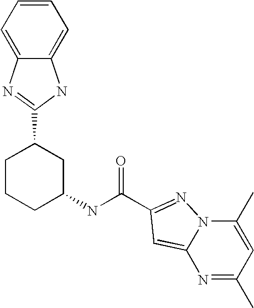 | HPLC Rt = 0.97 MS: [M + H] = 389.05 | 47 | 50 |
| 195 | N-[(1R,3S)-3- (1H- benzimidazol-2- yl)cyclohexyl] chromane-3- carboxamide triflouroacetate | 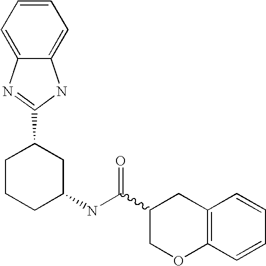 | HPLC Rt = 1.27 MS: [M + H] = 376.097 | 81 | 50 |
| 196 | N-[(1R,3S)-3- (1H- benzimidazol-2- yl)cyclohexyl]- 2,3-dihydro-1,4- benzodioxine- 2-carboxamide triflouroacetate | 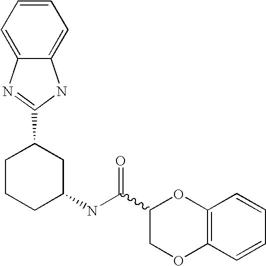 | HPLC Rt = 1.26 MS: [M + H] = 378.08 | 70 | 50 |
| 197 | N-[(1R,3S)-3- (1H- benzimidazol-2- yl)cyclohexyl]- 2-methyl-1H- benzimidazole- 5-carboxamide triflouroacetate | 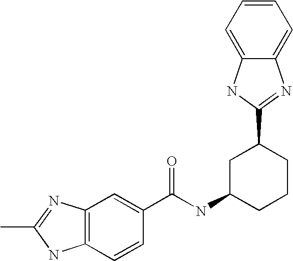 | HPLC Rt = 0.69 MS: [M + H] = 187.558 | 50 | 50 |
| 198 | N-[(1R,3S)-3- (1H- benzimidazol-2- yl)cyclohexyl]- 2,3-dihydro-1- benzofuran-5- carboxamide triflouroacetate | 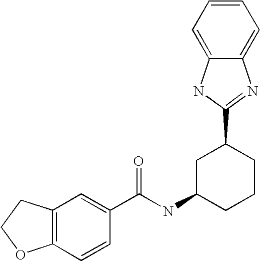 | HPLC Rt = 1.09 MS: [M + H] = 362.082 | 85 | 50 |
| 199 | N-[(1R,3S)-3- (1H- benzimidazol-2- yl)cyclohexyl]- 1H-indazole-3- carboxamide triflouroacetate | 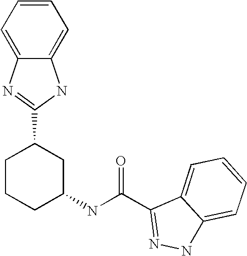 | HPLC Rt = 1.1 MS: [M + H] = 360.088 | 67 | 50 |
| 200 | N-[(1R,3S)-3- (1H- benzimidazol-2- yl)cyclohexyl]- 1-methyl- 1,2,3,4- tetrahydroquino- line-6- carboxamide HCl | 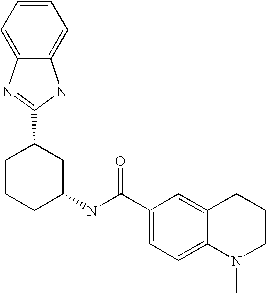 | HPLC Rt = 2.3 MS: [M + H] = 389.3 | 109.92 | 4 |
| 201 | N-[(1R,3S)-3- (1H- benzimidazol-2- yl)cyclohexyl]- 1-methyl- 1,2,3,4- tetrahydroquino- line-6- carboxamide triflouroacetate | 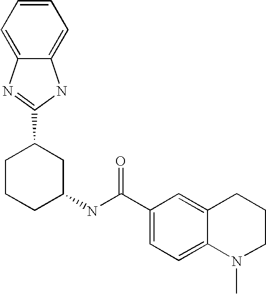 | HPLC Rt = 1.29 MS: [M + H] = 389.137 | 101 | 50 |
| 202 | N-[(1R,3S)-3- (1H- benzimidazol-2- yl)cyclohexyl]- 1,2,3- benzothiadiazole- 5- carboxamide triflouroacetate | 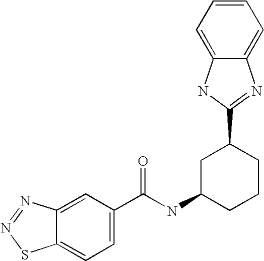 | HPLC Rt = 1.08 MS: [M + H] = 378.039 | 34 | 50 |
| 203 | N-[(1R,3S)-3- (1H- benzimidazol-2- yl)cyclohexyl]- 1H-indole-6- carboxamide triflouroacetate | 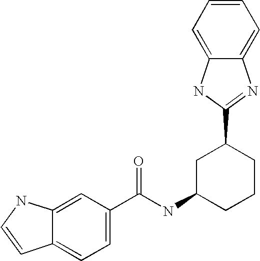 | HPLC Rt = 1.12 MS: [M + H] = 359.11 | 109 | 50 |
| 204 | N-[(1R,3S)-3- (1H- benzimidazol-2- yl)cyclohexyl]- 1,3-dimethyl- 1H-thieno[2,3- c]pyrazole-5- carboxamide triflouroacetate | 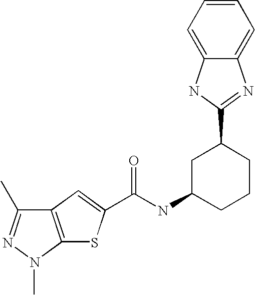 | HPLC Rt = 1.05 MS: [M + H] = 394.075 | 103 | 50 |
| 205 | N-[(1R,3S)-3- (1H- benzimidazol-2- yl)cyclohexyl]- 1H-indole-3- carboxamide triflouroacetate | 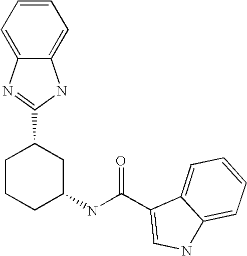 | HPLC Rt = 1.08 MS: [M + H] = 359.102 | 54 | 50 |
| 206 | N-[(1R,3S)-3- (1H- benzimidazol-2- yl)cyclohexyl]- 2,1,3- benzothiadiazole- 5- carboxamide triflouroacetate | 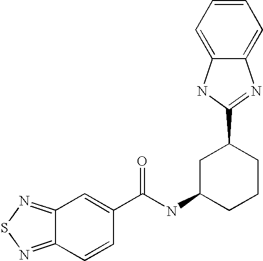 | HPLC Rt = 1.06 MS: [M + H] = 378.038 | 56 | 50 |
| 207 | (2R)-N- [(1R,3S)-3-(1H- benzimidazol-2- yl)cyclohexyl]- chromane-2- carboxamide triflouroacetate | 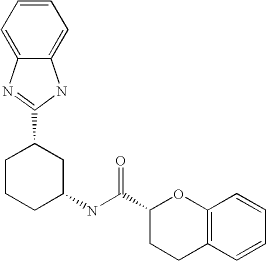 | HPLC Rt = 1.32 MS: [M + H] = 376.116 | 107 | 50 |
| 208 | N-[(1R,3S)-3- (1H- benzimidazol-2- yl)cyclohexyl]- 4-methoxy-1H- indole-2- carboxamide triflouroacetate | 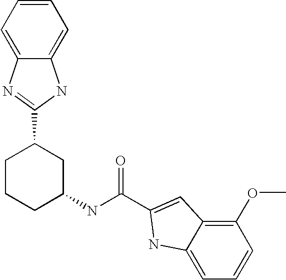 | HPLC Rt = 1.28 MS: [M + H] = 389.113 | 80 | 50 |
| 209 | N-[(1R,3S)-3- (1H- benzimidazol-2- yl)cyclohexyl]- 6-fluoro-1H- indole-2- carboxamide triflouroacetate | 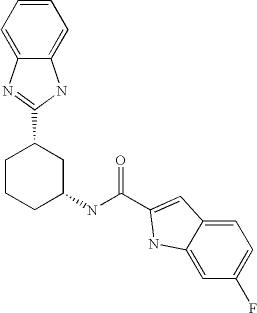 | HPLC Rt = 1.32 MS: [M + H] = 377.079 | 32 | 50 |
| 210 | N-[(1R,3S)-3- (1H- benzimidazol-2- yl)cyclohexyl]- 6-chloro-1H- indole-2- carboxamide triflouroacetate | 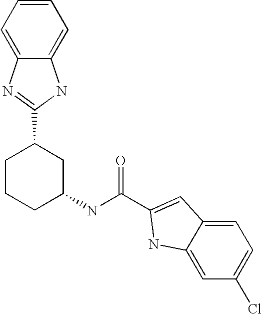 | HPLC Rt = 1.45 MS: [M + H] = 393.058 | 88 | 50 |
| 211 | N-[(1R,3S)-3- (1H- benzimidazol-2- yl)cyclohexyl]- 6-methoxy-1H- indole-2- carboxamide triflouroacetate | 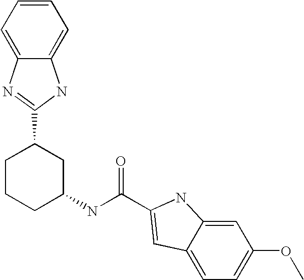 | HPLC Rt = 1.27 MS: [M + H] = 389.104 | 89 | 50 |
| 212 | N-[(1R,3S)-3- (1H- benzimidazol-2- yl)cyclohexyl]- 4,5,6,7,8,9- hexahydro-1H- cycloocta[c] pyrazole-3- carboxamide triflouroacetate | 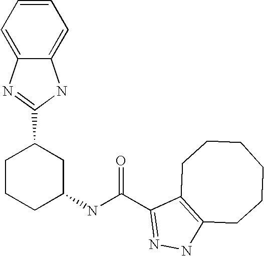 | HPLC Rt = 1.31 MS: [M + H] = 392.155 | 71 | 50 |
| 213 | N-[(1R,3S)-3- (1H- benzimidazol-2- yl)cyclohexyl]- 2-oxo-2,3- dihydro-1,3- benzoxazole-5- carboxamide triflouroacetate | 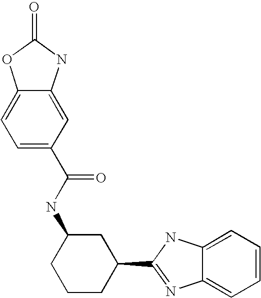 | HPLC Rt = 0.9 MS: [M + H] = 377.069 | 73 | 50 |
| 214 | N-[(1R,3S)-3- (1H- benzimidazol-2- yl)cyclohexyl]- 3-ethyl-1H- indole-2- carboxamide triflouroacetate | 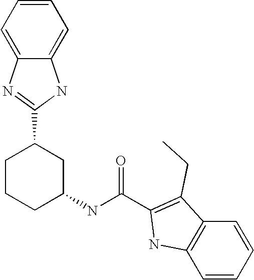 | HPLC Rt = 1.48 MS: [M + H] = 387.115 | 100 | 50 |
| 215 | N-[(1R,3S)-3- (1H- benzimidazol-2- yl)cyclohexyl]- 2-methyl-1,3- benzothiazole- 5-carboxamide triflouroacetate | 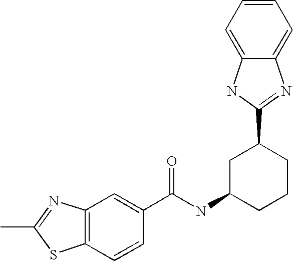 | HPLC Rt = 1.1 MS: [M + H] = 391.069 | 46 | 50 |
| 216 | N-[(1R,3S)-3- (1H- benzimidazol-2- yl)cyclohexyl]- 1,3- benzothiazole- 5-carboxamide triflouroacetate | 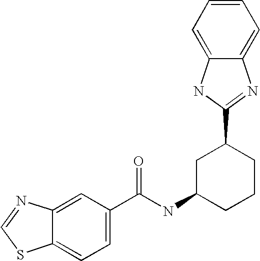 | HPLC Rt = 1.02 MS: [M + H] = 377.054 | 86 | 50 |
| 217 | N-[(1R,3S)-3- (1H- benzimidazol-2- yl)cyclohexyl]- 1-methyl-2-oxo- 2,3-dihydro-1H- benzimidazole- 5-carboxamide triflouroacetate | 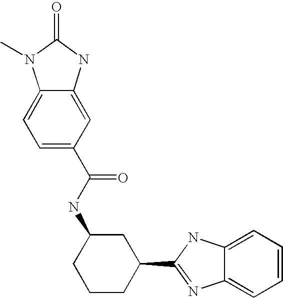 | HPLC Rt = 0.88 MS: [M + H] = 390.095 | 85 | 50 |
| 218 | (2S)-N- [(1R,3S)-3-(1H- benzimidazol-2- yl)cyclohexyl] chromane-2- carboxamide triflouroacetate | 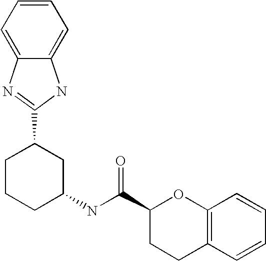 | HPLC Rt = 1.3 MS: [M + H] = 376.093 | 96 | 50 |
| 219 | N-[(1R,3S)-3- (1H- benzimidazol-2- yl)cyclohexyl]- 2-oxoindoline- 5-carboxamide triflouroacetate | 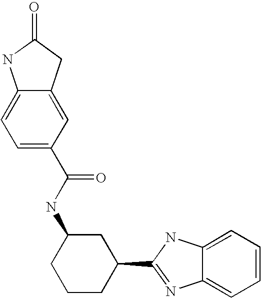 | HPLC Rt = 0.84 MS: [M + H] = 375.091 | 87 | 50 |
| 220 | N-[(1R,3S)-3- (1H- benzimidazol-2- yl)cyclohexyl]- 2-oxo-1,2,3,4- tetrahydroquin- oline-6- carboxamide triflouroacetate | 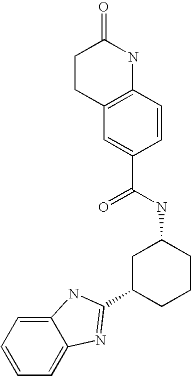 | HPLC Rt = 0.88 MS: [M + H] = 389.108 | 81 | 50 |
| 221 | N-[(1R,3S)-3- (1H- benzimidazol-2- yl)cyclohexyl]- 6- ethylimidazo[1, 2-a]pyridine-2- carboxamide triflouroacetate | 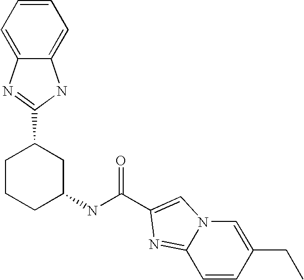 | HPLC Rt = 0.99 MS: [M + H] = 388.123 | 48 | 50 |
| 222 | N-[(1R,3S)-3- (1H- benzimidazol-2- yl)cyclohexyl]- 7-methoxy-1,3- benzodioxole- 5-carboxamide triflouroacetate | 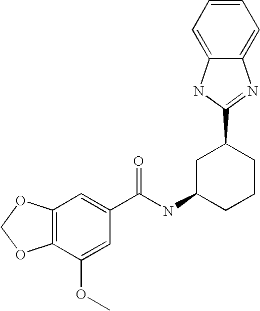 | HPLC Rt = 1.11 MS: [M + H] = 394.081 | 52 | 50 |
| 223 | N-((1R,3S)-3- [6- (trifluoromethyl)- 1H- benzimidazol-2- yl]cyclohexyl]- 2,3-dihydro-1,4- benzodioxine- 6-carboxamide HCl | 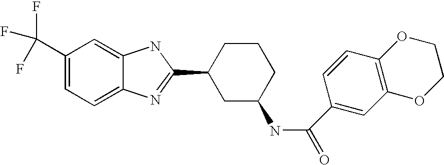 | HPLC Rt = 2.1 MS: [M + H] = 446.6 | 105 | 5 |
| 224 | N-[(1R,3S)-3- (6-bromo-1H- benzimidazol-2- yl)cyclohexyl]- 2,3-dihydro-1,4- benzodioxine- 6-carboxamide HCl | 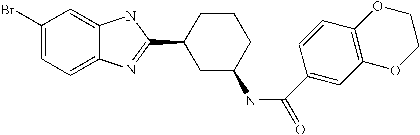 | 1H NMR (methanol-d₄, 400 MHz) 1.5-1.6 (m, 1H), 1.6-1.7 (m, 3H), 2.0- 2.1 (m, 2H), 2.2-2.25 (m, 1H), 2.45- 2.5 (m, 1H), 3.3-3.4 (m, 1H), 3.85 (s, 3H), 4.0-4.1 (m, 1H), 4.2 (s, 4H), 6.8 (d, 1H), 7.3- 7.35 (m, 2H), 7.6-7.7 (m, 2H), 7.9 (s, 1H); HPLC Rt = 1.8 MS: [M + H] = 356.4 | 106 | 5 |
| 225 | 1-((1R,3S)-3- (1H- benzo[d]imidaz- ol-2- yl)cyclohexyl)- 3-(3- chloropropyl)- urea | 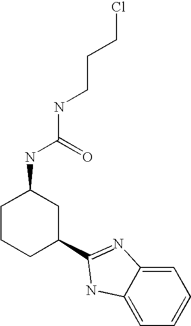 | HPLC Rt = MS: [M + H] = 335.2 | 70 | 47 |
| 226 | 1-((1R,3S)-3- (1H- benzo[d]imidaz- ol-2- yl)cyclohexyl)- 3- cyclopentylurea | 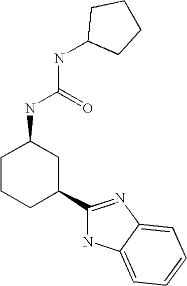 | HPLC Rt = 1.2 MS: [M + H] = 327.3 | 63 | 47 |
| 227 | 1-[(1R,3S)-3- (1H- benzimidazol-2- yl)cyclohexyl]- 3-(3,5- dichlorophenyl) urea | 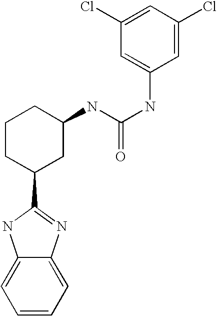 | HPLC Rt = 1.9 MS: [M + H] = 403.3 | 115 | 47 |
| 228 | 1-[(1R,3S)-3- (1H- benzimidazol-2- yl)cyclohexyl]- 3-(4- cyanophenyl) urea | 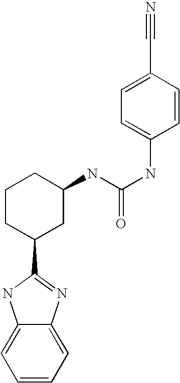 | HPLC Rt = 1.5 MS: [M + H] = 359.6 | 109 | 47 |
| 229 | 1-[(1R,3S)-3- (1H- benzimidazol-2- yl)cyclohexyl]- 3-(5-phenyl-2- thienyl)urea | 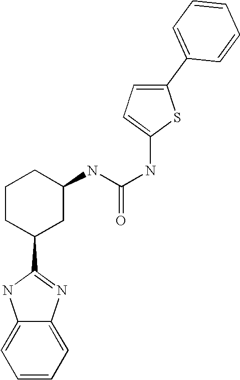 | HPLC Rt = 3.2 MS: [M + H] = 416.6 | 108 | 47 |
| 230 | 1-[(1R,3S)-3- (1H- benzimidazol-2- yl)cyclohexyl]- 3-(6-fluoro-4H- 1,3- benzodioxol-8- yl)urea | 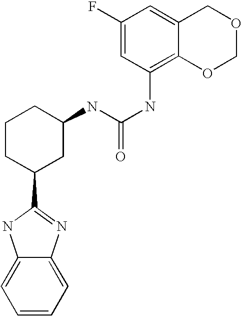 | HPLC Rt = 1.5 MS: [M + H] = 410.6 | 111 | 47 |
| 231 | 1-[(1R,3S)-3- (1H- benzimidazol-2- yl)cyclohexyl] 3-(3- cyanophenyl)- urea | 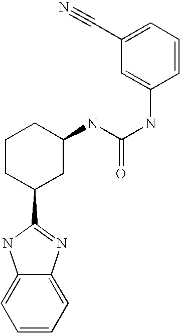 | HPLC Rt = 1.4 MS: [M + H] = 359.6 | 103 | 47 |
| 232 | 1-[(1R,3S)-3- (1H- benzimidazol-2- yl)cyclohexyl]- 3-(4- methoxyphenyl) urea | 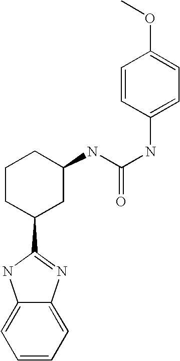 | HPLC Rt = 1.3 MS: [M + H] = 364.6 | 103 | 47 |
| 233 | 1-((1R,3S)-3- (1H- benzo[d]imidaz- ol-2- yl)cyclohexyl)- 3-(3,5- dimethylisoxaz- ol-4-yl)urea | 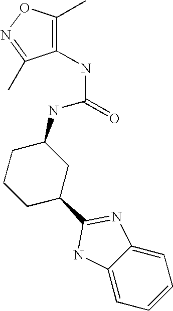 | HPLC Rt = 0.9 MS: [M + H] = 353.6 | 48 | 47 |
| 234 | 1-[(1R,3S)-3- (1H- benzimidazol-2- yl)cyclohexyl]- 3-(2,4- dichlorophenyl) urea | 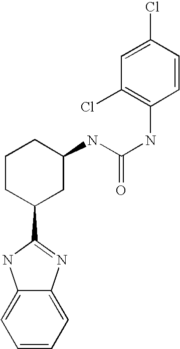 | HPLC Rt = 1.8 MS: [M + H] = 403.3 | 108 | 47 |
| 235 | 1-[(1R,3S)-3- (1H- benzimidazol-2- yl)cyclohexyl]- 3-(4-methoxy- 2- methylphenyl) urea | 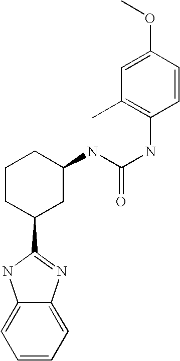 | HPLC Rt = 1.3 MS: [M + H] = 379.2 | 98 | 47 |
| 236 | 1-[(1R,3S)-3- (1H- benzimidazol-2- yl)cyclohexyl]- 3-(4- isopropylphenyl) urea | 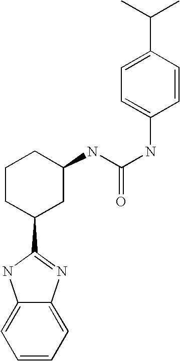 | HPLC Rt = 1.8 MS: [M + H] = 376.6 | 108 | 47 |
| 237 | 1-[(1R,3S-3- (1H- benzimidazol-2- yl)cyclohexyl]- 3-(3,4- dichlorophenyl) urea | 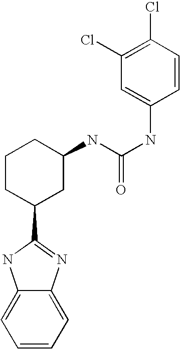 | HPLC Rt = 1.9 MS: [M + H] = 403.3 | 111 | 47 |
| 238 | 1-[(1R,3S)-3- (1H- benzimidazol-2- yl)cyclohexyl]- 3-[4-chloro-3- (trifluoromethyl) phenyl]urea | 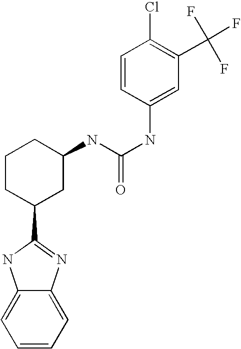 | HPLC Rt = 2.1 MS: [M + H] = 436.5 | 114 | 47 |
| 239 | N-((1S,3R)-3- (5-methoxy-1H- benzo[d]imidaz- ol-2- yl)cyclohexyl)- 2,3- dihydrobenzo[b] [1,4]dioxine-6- carboxamide HCl | 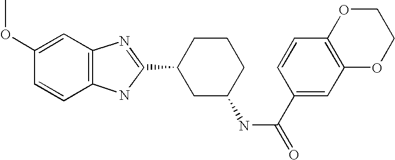 | HPLC Rt = 1.3 MS: [M + H] = 408.1 | 23 | 4 |
| 240 | N-([1R,3R]-3- (1-methyl-1H- benzo[d]imidaz- ol-2- yl)cyclohexyl)- 2,3- dihydrobenzo[b] [1,4]dioxine-6- carboxamide HCl | 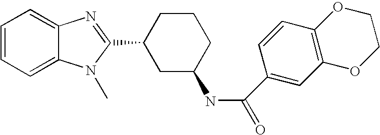 | HPLC Rt = 1.7 MS: [M + H] = 392.2 | 60 | 3 |
| 241 | N-([1R,3S)]-3- (1H- benzo[d]imidaz- ol-2- yl)cyclohexyl)- 2,3- dihydrobenzo[b] [1,4]dioxine-6- carboxamide HCl | 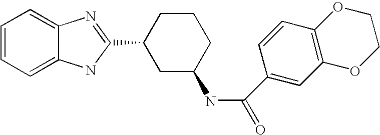 | HPLC Rt = 1.6 MS: [M + H] = 378.2 | 63 | 2 |
| 242 | N-((1R,3S)-3- (1H- benzo[d]imidaz- ol-2- yl)cyclohexyl)- 2,3-dihydro- [1,4]dioxino[2,3- b]pyridine-6- carboxamide | 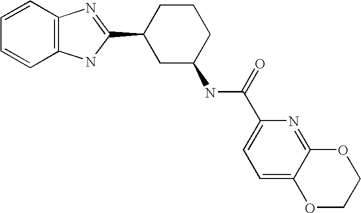 | HPLC Rt = 1.6 MS: [M + H] = 379.3 | 86 | 50 |
| 243 | N-((1R,3S)-3- (1H- benzo[d]imidaz- ol-2- yl)cyclohexyl)- 2,3-dihydro- [1,4]dioxino[2,3- b]pyridine-7- carboxamide | 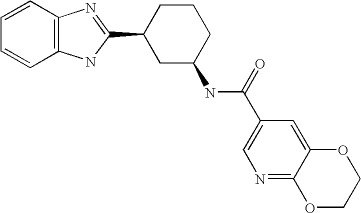 | HPLC Rt = 1.6 MS: [M + H] = 379.3 | 96 | 50 |
| 244 | N-(5-(1-methyl- 1H- benzo[d]imidaz- ol-2- yl)bicyclo[3.1.1] heptan-1-yl)- 2,3- dihydrobenzo[b] [1,4]dioxine-6- carboxamide | 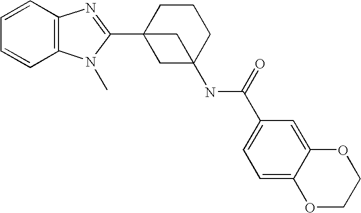 | HPLC Rt = 1.3 MS: [M + H] = 404.3 | 66 | 51 |
| 245 | 3,5-dimethoxy- N-(5-(1-methyl- 1H- benzo[d]imidaz- ol-2- yl)bicyclo[3.1.1] heptan-1- yl)benzamide | 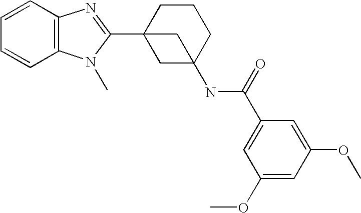 | HPLC Rt = 1.7 MS: [M + H] = 406.3 | 49 | 51 |
| 246 | N-(3-(1H- benzo[d]imidaz- ol-2- yl)cyclohexyl)- 3-cyclopropyl- 1H-pyrazole-5- carboxamide HCl | 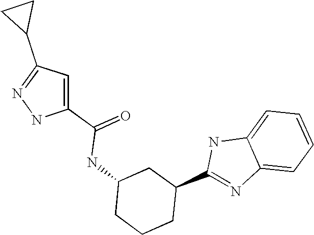 | HPLC Rt = 1.9 MS: [M + H] = 350.2 | 44 | 48 |
| 247 | N-(3-(1H- benzo[d]imidaz- ol-2- yl)cyclohexyl)- 2-oxo-1,2,3,4- tetrahydroquino- line-6- carboxamide HCl | 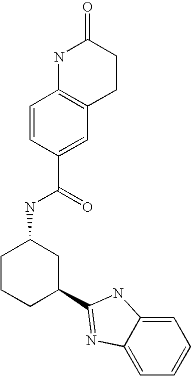 | HPLC Rt = 1.8 MS: [M + H] = 389.2 | 98 | 48 |
| 248 | N-(3-(1H- benzo[d]imidaz- ol-2- yl)cyclohexyl)- 6-methoxy-1- methyl-1H- indole-2- carboxamide HCl | 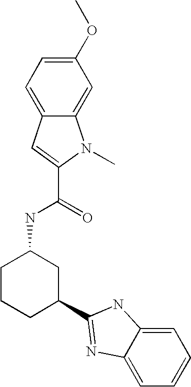 | HPLC Rt = 2.5 MS: [M + H] = 403.2 | 74 | 48 |
| 249 | N-(3-(1H- benzo[d]imidaz- ol-2- yl)cyclohexyl)- 4-methyl-3-oxo- 3,4-dihydro-2H- benzo[d][1,4]ox- azine-6- carboxamide HCl | 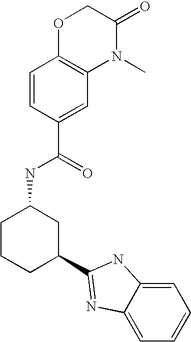 | 1H NMR (methanol-d₄, 400 MHz) 1.7-1.8 (m, 1H), 1.8-1.85 (m, 2H), 1.85-1.95 (m, 1H), 2.05- 2.1 (m, 1H), 2.15-2.2 (m, 1H), 2.2-2.3 (m, 1H), 2.35-2.4 (m, 1H), 3.2 (s, 3H), 3.65- 3.7 (m, 1H), 4.15-4.2 (m, 1H), 4.65 (s, 2H), 7.0 (d, 1H), 7.5-7.6 (m, 4H), 7.7- 7.75(m, 2H); HPLC Rt = 2 MS: [M + H] = 405.2 | 109 | 48 |
| 250 | 2,2-difluoro-N- (5-(1-methyl- 1H- benzo[d]imidaz- ol-2- yl)bicyclo[3.1.1] heptan-1- yl)benzo[d][1,3] dioxole-5- carboxamide | 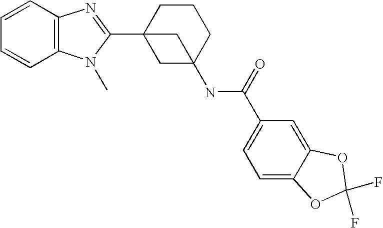 | HPLC Rt = 1.98 MS: [M + H] = 426.4 | 94 | 51 |
| 251 | N-((1R,3S)-3- (1H- benzo[d]imidaz- ol-2- yl)cyclohexyl)- 2,2- difluorobenzo[d] [1,3]dioxole-5- carboxamide | 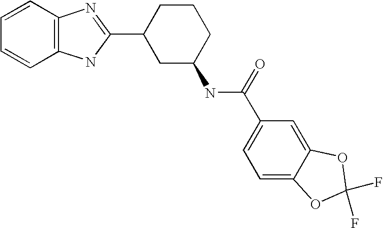 | HPLC Rt = 1.8 MS: [M + H] = 400.3 | 102 | 50 |
| 252 | N-(3-(1H- benzo[d]imidaz- ol-2- yl)cyclohexyl)- 3,5- dimethoxybenz- amide HCl | 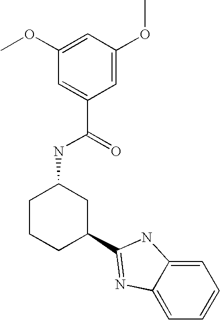 | HPLC Rt = 2.1 MS: [M + H] = 380.2 | 46 | 48 |
| 253 | N-((1R,3S)-3- (1H- benzo[d]imidaz- ol-2- yl)cyclohexyl)- 2,3-dihydro- [1,4]dioxino[2,3- c]pyridine-7- carboxamide | 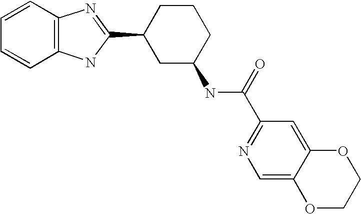 | HPLC Rt = 1.4 MS: [M + H] = 374.3 | 100 | 50 |
| 254 | N-(3-(5- methoxy-1H- benzo[d]imidaz- ol-2- yl)cyclohexyl)- 2,3- dihydrobenzo[b] [1,4]dioxine-6- carboxamide HCl | 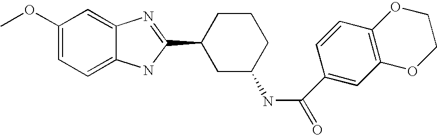 | HPLC Rt = 2.3 MS: [M + H] = 408.1 | 91 | 4 |
| 255 | 1-(5-(1H- benzo[d]imidaz- ol-2- yl)bicyclo[3.1.1] heptan-1-yl)-3- (4-fluoro-3- (trifluoromethyl) phenyl)urea HCL | 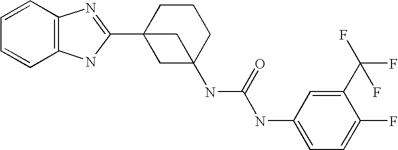 | HPLC Rt = 1.4 MS: [M + H] = 433.2 | 101 | 47 |
| 256 | 1-(5-(1H- benzo[d]imidaz- ol-2- yl)bicyclo[3.1.1] heptan-1-yl)-3- (4- cyanophenyl)- urea HCl | 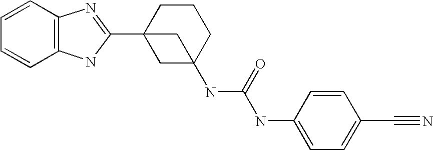 | HPLC Rt = 1.7 MS: [M + H] = 372.2 | 94 | 47 |
| 257 | N-{5-[5- (trifluoromethyl)- 1H- benzimidazol-2- yl]bicyclo[3.1.1] hept-1-yl}-2,3- dihydro-1,4- benzodioxine- 6-carboxamide | 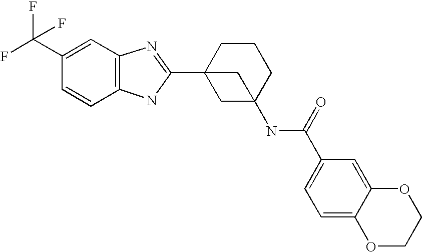 | HPLC Rt = 2.0 MS: [M + H] = 456.2 | 106.75 | 51 |
| 258 | N-{5-[5- (trifluoromethyl)- 1H- benzimidazol-2- yl]bicyclo[3.1.1] hept-1-yl}-2,3- dihydro-1,4- benzodioxine- 6-carboxamide HCL | 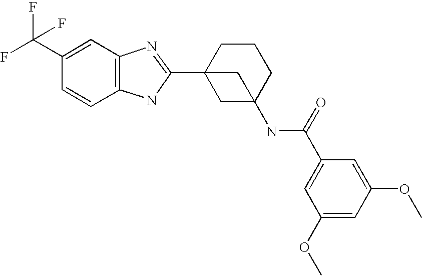 | HPLC Rt = 2.0 MS: [M + H] = 458.2 | 99.22 | 51 |
| 259 | 3,5-dimethoxy- N-{5-[5- (trifluoromethyl)- 1H- benzimidazol-2- yl)bicyclo[3.1.1] hept-1- yl}benzamide | 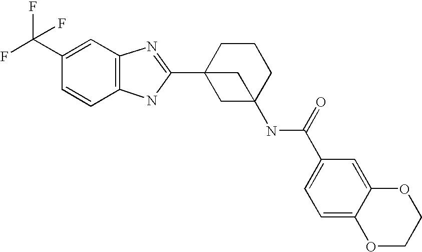 | HPLC Rt = 2.1 MS: [M + H] = 460.2 | 95.48 | 51 |
| 260 | 3,5-dimethoxy- N-{5-[5- (trifluoromethyl)- 1H- benzimidazol-2- yl]bicyclo[3.1.1] hept-1- yl}benzamide HCl | 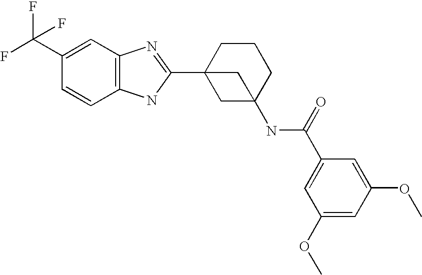 | HPLC Rt = 2.1 MS: [M + H] = 460.2 | 101.34 | 51 |
| 261 | 1-(4- cyanophenyl)- 3-{5-[5- (trifluoromethyl)- 1H- benzimidazol-2- yl]bicyclo[3.1.1] hept-1-yl}urea | 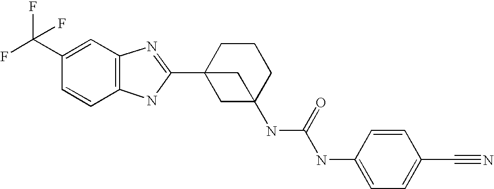 | HPLC Rt = 2.2 MS: [M + H] = 440.2 | 78.8 | 47 |
| 262 | 1-(4- cyanophenyl)- 3-{5-[5- (trifluoromethyl)- 1H- benzimidazol-2- yl]bicyclo[3.1.1] hept-1-yl}urea HCl | 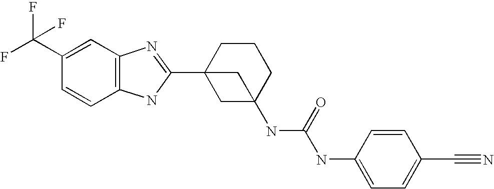 | HPLC Rt = 2.2 MS: [M + H] = 440.2 | 89 | 47 |
| 263 | N-{5-[5- (trifluoromethyl)- 1H- benzimidazol-2- yl]bicyclo[3.1.1] hept-1-yl}-2,3- dihydro[1,4]di- oxino[2,3- c]pyridine-7- carboxamide | 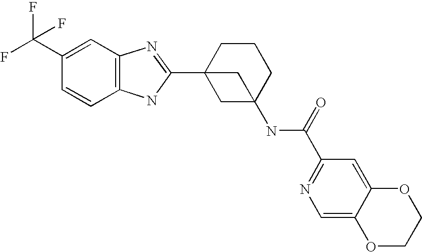 | HPLC Rt = 2.1 MS: [M + H] = 459.2 | 91.36 | 50 |
| 264 | N-{5-[5- (trifluoromethyl)- 1H- benzimidazol-2- yl]bicyclo[3.1.1] hept-1-yl}-2,3- dihydro[1,4]di- oxino[2,3- c]pyridine-7- carboxamide HCl | 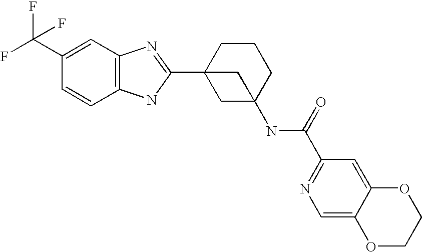 | HPLC Rt = 2.1 MS: [M + H] = 459.2 | 94.31 | 50 |
| 265 | N-[(1R,3S)-3- (1H- benzimidazol-2- yl)cyclohexyl] chromane-6- carboxamide | 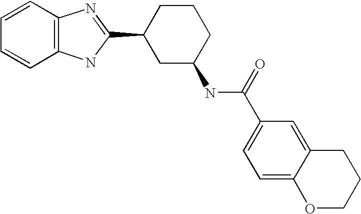 | HPLC Rt = 1.6 MS: [M + H] = 376.3 | 102.75 | 50 |
| 266 | N-[(1R,3S)-3- (1H- benzimidazol-2- yl)cyclohexyl] chromane-6- carboxamide HCl | 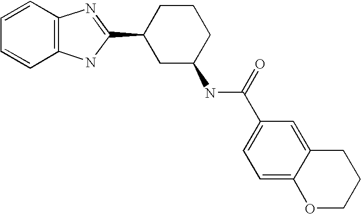 | HPLC Rt = 1.6 MS: [M + H] = 376.3 | 102.05 | 50 |
| 267 | N-[(1R,3S)-3- (1H- benzimidazol-2- yl)cyclohexyl]- 2- (cyclopropyl- methyl)-1,2,3,4- tetrahydroiso- quinoline-7- carboxamide HCl | 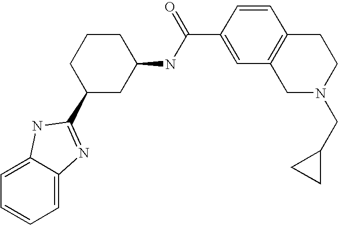 | HPLC Rt = 1.9 MS: [M + H] = 429.3 | 103.43 | 4 |
| 268 | (2S,3aS,7aS)- N-[(1R,3S)-3- (1H- benzimidazol-2- yl)cyclohexyl]- 1- methyloctahy- dro-1H-indole-2- carboxamide HCl | 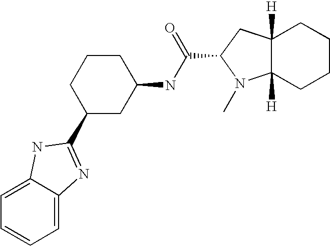 | HPLC Rt = 2.0 MS: [M + H] = 381.3 | 50.7 | 4 |
| 269 | N-[5-(5-chloro- 1H- benzimidazol-2- yl)bicyclo[3.1.1] hept-1-yl]-3,5- dimethoxybenz- amide | 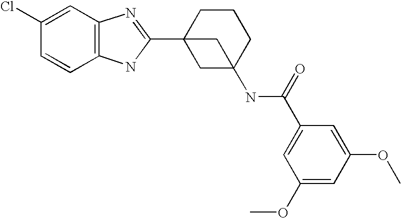 | HPLC Rt = 1.9 MS: [M + H] = 426.2 | 107.23 | 51 |
| 270 | 1-[5-(5-chloro- 1H- benzimidazol-2- yl)bicyclo[3.1.1] hept-1-yl]-3-(4- cyanophenyl) urea | 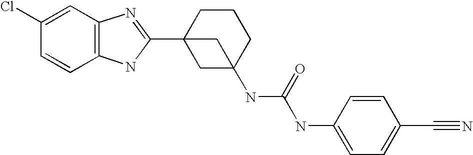 | HPLC Rt = 1.9 MS: [M + H] = 406.2 | 106.73 | 47 |
| 271 | N-[5-(5-chloro- 1H- benzimidazol-2- yl)bicyclo[3.1.1] hept-1-yl]-2,3- dihydro[1,4]di- oxino[2,3- c]pyridine-7- carboxamide | 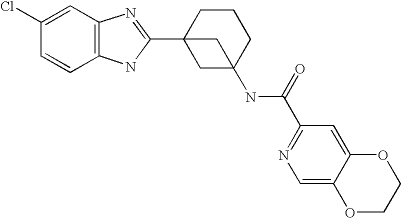 | HPLC Rt = 1.8 MS: [M + H] = 425.2 | 84.7 | 50 |
| 272 | N-[5-(5-chloro- 1H- benzimidazol-2- yl)bicyclo[3.1.1] hept-1-yl]-2,3- dihydro-1,4- benzodioxine- 6-carboxamide | 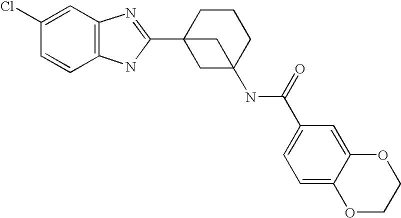 | HPLC Rt = 1.9 MS: [M + H] = 424.2 | 110.5 | 51 |
| 273 | 4-azido-N-{5- [5- (trifluoromethyl)- 1H- benzimidazol-2- yl]bicyclo[3.1.1] hept-1- yl}benzamide | 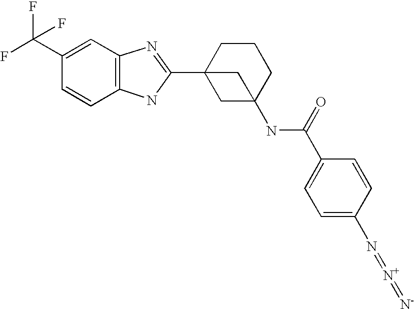 | HPLC Rt = 2.3 MS: [M + H] = 441.2 | 93.23 | 50 |
| 274 | 4- (trifluoroacetyl)- N-{5-[5- (trifluoromethyl)- 1H- benzimidazol-2- yl]bicyclo[3.1.1] hept-1- yl}benzamide | 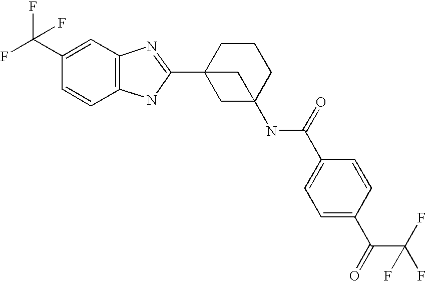 | HPLC Rt = 2.0 MS: [M + H] = 494.3 | 104.72 | 50 |
| 275 | 4-benzoyl-N-{5- [5- (trifluoromethyl)- 1H- benzimidazol-2- yl]bicyclo[3.1.1] hept-1- yl}benzamide | 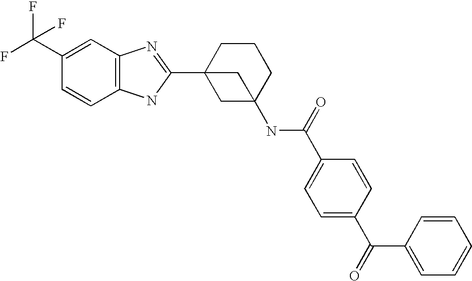 | HPLC Rt = 2.5 MS: [M + H] = 504.2 | 89 | 50 |
| 276 | N-{5-[5- (trifluoromethyl)- 1H- benzimidazol-2- yl]bicyclo[3.1.1] hept-1- yl}chromane-6- carboxamide | 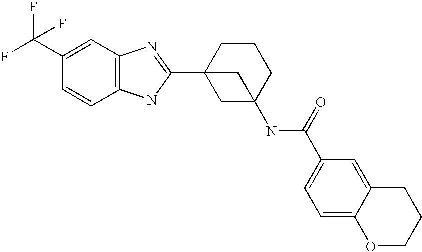 | HPLC Rt = 2.2 MS: [M + H] = 456.2 | 84.7 | 50 |
| 277 | 4-azido-N-[5- (1H- benzimidazol-2- yl)bicyclo[3.1.1] hept-1- yl]benzamide | 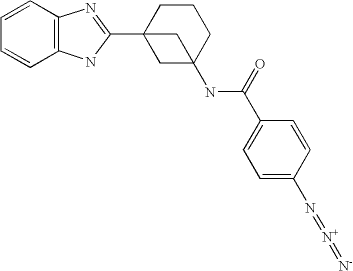 | HPLC Rt = 1.6 MS: [M + H] = 373.3 | 82.3 | 50 |
| 278 | N-[5-(1H- benzimidazol-2- yl)bicyclo[3.1.1] hept-1-yl]-4- benzoylbenz- amide | 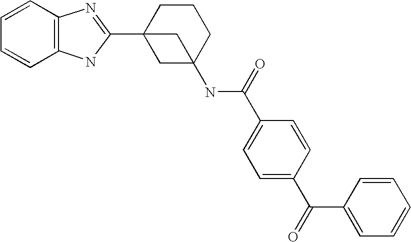 | HPLC Rt = 2.0 MS: [M + H] = 436.2 | 99.851 | 50 |
| 279 | N-[5-(1H- benzimidazol-2- yl)bicyclo[3.1.1] hept-1- yl]chromane-6- carboxamide | 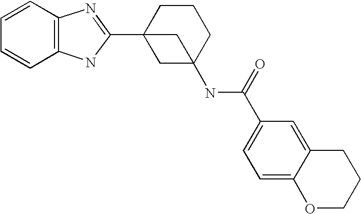 | HPLC Rt = 1.4 MS: [M + H] = 388.3 | 92.59 | 50 |
| 280 | 4-methyl-3-oxo- N-{(1R,3S)-3- [5- (trifluoromethyl)- 1H- benzimidazol-2- yl]cyclohexyl}- 3,4-dihydro-2H- 1,4- benzoxazine-6- carboxamide HCl | 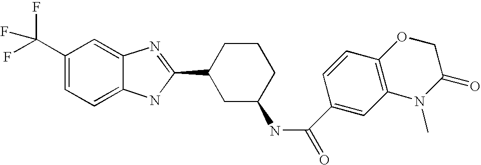 | 1H NMR (methanol- d4, 400 MHz) d 1.45- 1.55 (m, 1H), 1.6-1.7 (m, 2H), 1.7-1.8 (m, 1H), 2.0- 2.1 (m, 1H), 2.15-2.2 (m, 2H), 2.35- 2.4 (m, 1H), 3.1-3.2 (m, 1H), 3.3 (s, 3H), 4.0-4.1 (m,1 H), 4.65 (s, 2H), 7.0 (d, 1H), 7.45 (d, 1H), 7.55 (d, 1H), 7.6(s, 1H), 7.65 (d, 1H), 7.8 (s, 1H); HPLC Rt = 2.0 MS: [M + H] = 473.2 | 112 | 4 |
| 281 | N-{(1R,3S)-3- [5- (trifluoromethyl)- 1H- benzimidazol-2- yl]cyclohexyl]- 4,5,6,7- tetrahydro-1H- indazol-3- carboxamide HCl | 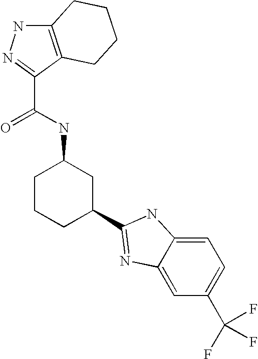 | HPLC Rt = 2.0 MS: [M + H] = 432.2 | 98.46 | 4 |
| 282 | 2-(6-methyl-2- oxopyridin- 1(2H)-yl)-N- {(1R,3S)-3-[5- (trifluoromethyl)- 1H- benzimidazol-2- yl]cyclohexyl} acetamide HCl | 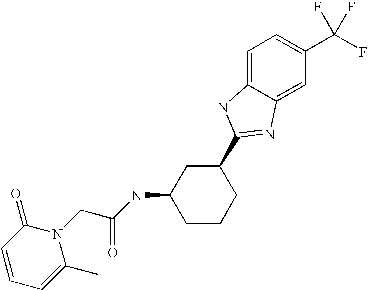 | HPLC Rt = 1.5 MS: [M + H] = 433.2 | 85.2 | 4 |
| 283 | N-{(1R,3S)-3- [5- (trifluoromethyl)- 1H- benzimidazol-2- yl]cyclohexyl} 4,5,6,7,8,9- hexahydro-1H- cycloocta[c]- pyrazole-3- carboxamide HCl | 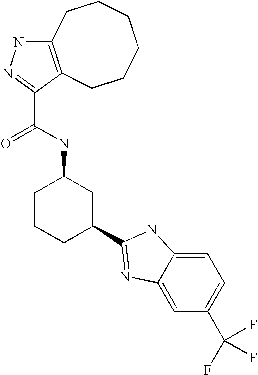 | HPLC Rt = 2.3 MS: [M + H] = 460.3 | 105.84 | 4 |
| 284 | 1,3-dimethyl-N- {(1R,3S)-3-[5- (trifluoromethyl)- 1H- benzimidazol-2- yl]cyclohexyl}- 1H-thieno[2,3- c]pyrazole-5- carboxamide HCl | 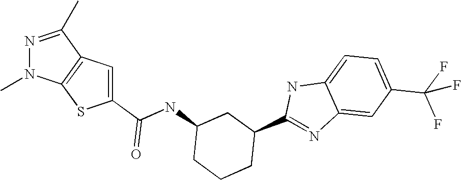 | HPLC Rt = 2.1 MS: [M + H] = 462.2 | 94.87 | 4 |
| 285 | 2-oxo-N- {(1R,3S)-3-[5- (trifluoromethyl)- 1H- benzimidazol-2- yl]cyclohexyl}- 1,2,3,4- tetrahydroquino- line-6- carboxamide HCl | 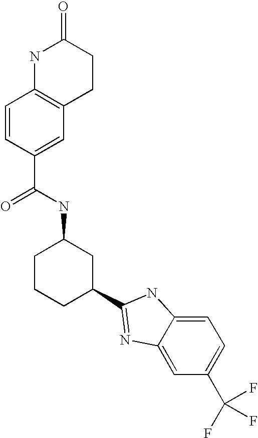 | HPLC Rt = 1.8 MS: [M + H] '2 457.2 | 110.8 | 4 |
| 286 | 2-oxo-N- {(1R,3S)-3-[5- (trifluoromethyl)- 1H- benzimidazol-2- yl]cyclohexyl} indoline-5- carboxamide HCl | 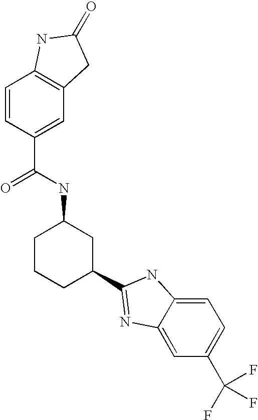 | HPLC Rt = 1.7 MS: [M + H] = 443.2 | 114.1 | 4 |
| 287 | N-{(1R,3S)-3- [5- (trifluoromethyl)- 1H- benzimidazol-2- yl]cyclohexyl}- 1,2,3,4- tetrahydroisoquino- line-6- carboxamide HCl | 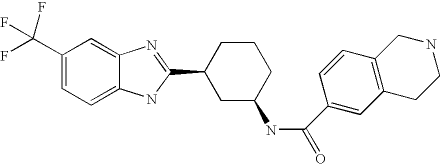 | HPLC Rt = 2.0 MS: [M + H] = 443.2 | 107.35 | 4 |
| 288 | 2,3,4,5,6- pentafluoro-N- {5-[5- (trifluoromethyl)- 1H- benzimidazol-2- yl]bicyclo[3.1.1] hept-1- yl}benzamide | 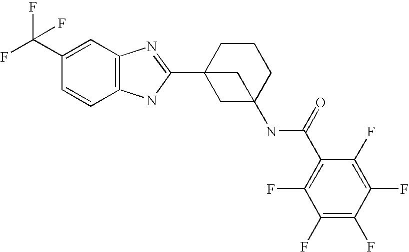 | HPLC Rt = 2.4 MS: [M + H] = 490.1 | 93.34 | 51 |
| 289 | 3,5-dimethoxy- N-{(1R,3S)-3- [5- (methylsulfonyl)- 1H- benzimidazol-2- yl]cyclohexyl} benzamide (+)- tartrate | 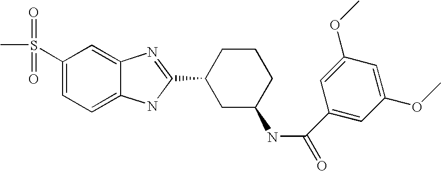 | HPLC Rt = 1.7 MS: [M + H] = 458.1 | 81 | 4 |
| 290 | 2- (cyclopropyl- methyl)-N- {(1R,3S)-3-[5- (trifluoromethyl)- 1H- benzimidazol-2- yl]cyclohexyl}- 1,2,3,4- tetrahydroisoquino- line-7- carboxamide HCl | 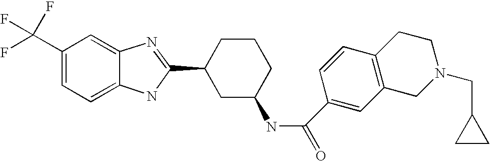 | HPLC Rt = 2.2 MS: [M + H] = 497.3 | 104.33 | 4 |
| 291 | N-[(1S,3S)-3- (1H- benzimidazol-2- yl)cyclohexyl]- 2- (cyclopropyl- methyl)-1,2,3,4- tetrahydroisoquino- line-7- carboxamide HCl | 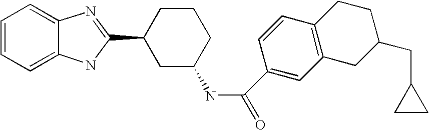 | HPLC Rt = 1.8 MS: [M + H] = 429.3 | 85.2 | 4 |
| 292 | 6-[(2- methoxyethyl) amino]-N- {(1R,3S)-3-[5- (trifluoromethyl)- 1H- benzimidazol-2- yl]cyclohexyl} nicotinamide HCl | 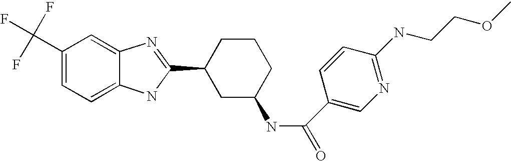 | 1H NMR (methanol d4, 400 MHz) d 1.45- 1.55 (m, 1H), 1.6-1.7 (m, 2H), 1.7-1.8 (m, 1H), 2.0- 2.1 (m, 1H), 2.15-2.2 (m, 2H), 2.35- 2.4 (m, 1H), 3.1-3.2 (m, 1H), 3.35 (s, 3H), 3.5-3.6 (m, 4H), 4.0- 4.1 (m, 1H), 6.55 (d, 1H), 7.45 (d, 1H), 7.65 (d, 1H), 7.8 (s, 1H), 7.85(d, 1H), 8.45 (s, 1H); HPLC Rt = 2.3 MS: [M + H] = 462.2 | 103.61 | 41 |
| 293 | 6-{[2- (dimethylamino) ethyl]amino}- N-{(1R,3S)-3- [5- (trifluoromethyl)- 1H- benzimidazol-2- yl]cyclohexyl}- nicotinamide HCl | 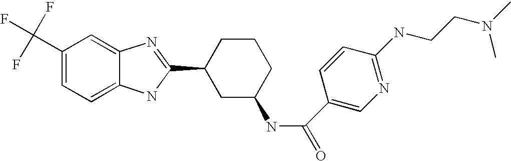 | HPLC Rt = 2.1 MS: [M + H] = 475.3 | 105.55 | 41 |
| 294 | N-[(1R)-3-(1H- benzimidazol-2- yl)cyclohexyl]- 3-oxo-3,4- dihydro-2H-1,4- benzoxazine-6- carboxamide (+)-tartrate | 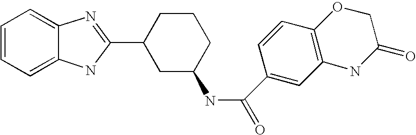 | HPLC Rt = 2.1 MS: [M + H] = 475.3 | 62.5 | |
| 295 | 2- (cyclopropyl- methyl)-N- {(1R,3S)-3-[5- (trifluoromethyl)- 1H- benzimidazol-2- yl]cyclohexyl}- 1,2,3,4- tetrahydroquino- line-6- carboxamide (+)-tartrate | 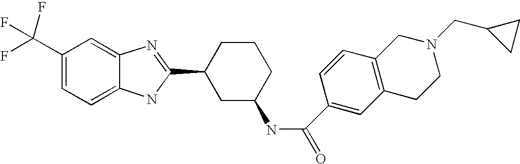 | HPLC Rt = 2.2 MS: [M + H] = 497.3 | 105.11 | 4 |
| 296 | N-[(1R,3S)-3- (5-chloro-1H- benzimidazol-2- yl)cyclohexyl]- 2- (cyclopropyl- methyl)-1,2,3,4- tetrahydroisoquino- line-7- carboxamide (+)-tartrate | 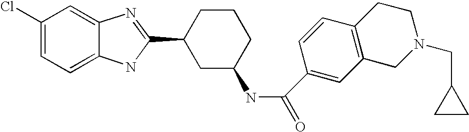 | HPLC Rt = 2.2 MS: [M + H] = 463.3 | 106.4 | 4 |
| 297 | 4-methyl-N- [(1R,3S)-3-(1- methyl-1H- benzimidazol-2- yl)cyclohexyl]- 3-oxo-3,4- dihydro-2H-1,4- benzoxazine-6- carboxamide (+)-tartrate | 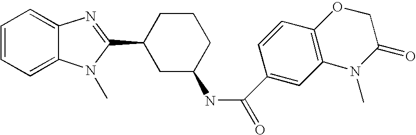 | HPLC Rt = 1.8 MS: [M + H] = 419.3 | 106.25 | 3 |
| 298 | N-[(1R,3S)-3- (5-chloro-1H- benzimidazol-2- yl)cyclohexyl]- 3,5- dimethoxybenz- amide (+)- tartrate | 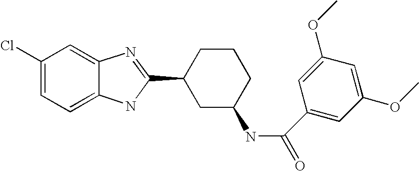 | HPLC Rt = 2.7 MS: [M + H] = 414.2 | 103.56 | 4 |
| 299 | N-[(1R,3S)-3- (5-chloro-1H- benzimidazol-2- yl)cyclohexyl]- 3-oxo-3,4- dihydro-2H-1,4- benzoxazine-6- carboxamide (+)-tartrate | 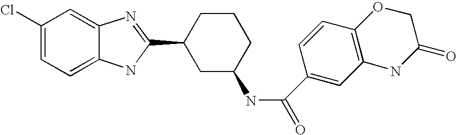 | HPLC Rt = 2.3 MS: [M + H] = 425.2 | 105.45 | 4 |
| 300 | N-[(1R,3S)-3- (5-chloro-1H- benzimidazol-2- yl)cyclohexyl]- 2-oxo-1,2,3,4- tetrahydroquino- line-6- carboxamide (+)-tartrate | 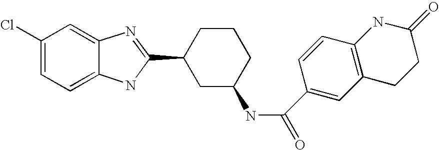 | 1H NMR (methanol- d4, 400 MHz) d 1.45- 1.55 (m, 1H), 1.6-1.7 (m, 2H), 1.7-1.8 (m, 1H), 2.0- 2.1 (m, 2H), 2.15-2.2 (m, 1H), 2.35- 2.4 (m, 1H), 2.55-2.65 (m, 2H), 2.90- 3.0 (m, 2H), 3.05-3.15 (m, 1H), 4.0-4.1 (m, 1H), 6.85 (d, 1H), 7.2 (d, 1H), 7.45-7.5 (m, 2H), 7.65 (s, 1H),7.95 (s, 1H); HPLC Rt = 2.3 MS: [M + H] = 423.2 | 105.8 | 4 |
| 301 | N-[(1R,3S)-3- (5-chloro-1H- benzimidazol-2- yl)cyclohexyl]- 4-methyl-3-oxo- 3,4-dihydro-2H- 1,4- benzoxazine-6- carboxamide (+)-tartrate | 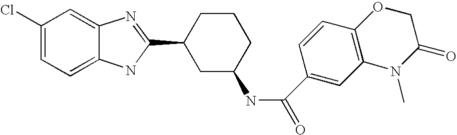 | HPLC Rt = 2.4 MS: [M + H] = 439.2 | 107.79 | 4 |
| 302 | 4-azido-2,3,5,6- tetrafluoro-N- {5-[5- (trifluoromethyl)- 1H- benzimidazol-2- yl]bicyclo[3.1.1] hept-1- yl}benzamide | 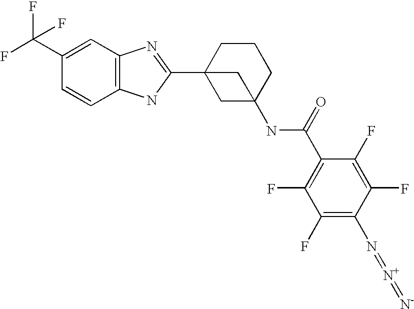 | HPLC Rt = 2.4 MS: [M + H] = 513.1 | 97.08 | 51 |
| 303 | N-{(1R,3S)-3- [5- (trifluoromethyl)- 1H- benzimidazol-2- yl]cyclohexyl}- 1,2,3,4- tetrahydroiso- quinoline-7- carboxamide HCl | 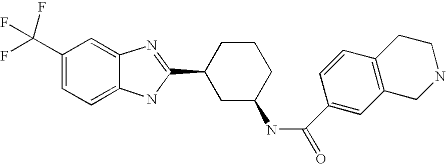 | 1H NMR (methanol- d4, 400 MHz) d 1.55- 1.65 (m, 1H), 1.65-1.75 (m, 2H), 1.75- 1.85 (m, 1H), 2.0-2.1 (m, 2H), 2.25-2.3 (m, 1H), 2.5- 2.55 (m, 1H), 3.15-3.2 (m, 2H), 3.4- 3.5 (m, 1H), 3.50-3.55 (m, 2H), 4.1- 4.15 (m, 1H)l, 4.4 (s, 1H), 7.35 (d, 1H), 7.7-7.8 (m,2H), 7.85 (d, 1H), 7.95 (d, 1H), 8.1 (s, 1H); HPLC Rt = 0.9 MS: [M + H] = 443.2 | 111.2 | 4 |
| 304 | 1-methyl-2-oxo- N-[(1R,3S)-3- [5- (trifluoromethyl)- 1H-benzimidazol-2- yl]cyclohexyl}- 1,2,3,4- tetrahydroquin- oline-6- carboxamide (+)-tartrate | 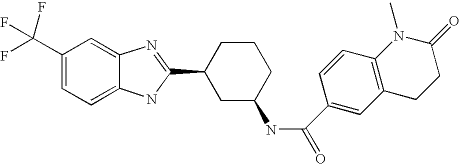 | 1H NMR (methanol- d4, 400 MHz) d 1.45- 1.55 (m, 1H), 1.6-1.7 (m, 2H), 1.7-1.8 (m, 1H), 2.0- 2.1 (m, 2H), 2.15-2.2 (m, 1H), 2.35- 2.4 (m, 1H), 2.55-2.65 (m, 2H), 2.90- 3.0 (m, 2H), 3.05-3.15 (m, 1H), 3.3 (s, 3H), 4.0-4.1 (m, 1H), 7.15 (d, 1H), 7.45 (d, 1H), 7.6 (d, 1H), 7.7 (s, 1H), 7.75-7.8 (m, 2H); HPLC Rt = 2.1 MS: [M + H] = 471.2 | 110.6 | 4 |
| 305 | N-[(1R,3S)-3- (5-chloro-1H- benzimidazol-2- yl)cyclohexyl]- 1-methyl-2-oxo- 1,2,3,4- tetrahydroquino- line-6- carboxamide (+)-tartrate | 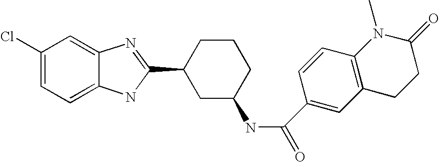 | HPLC Rt = 1.7 MS: [M + H] = 437.2 | 81.2 | 4 |
| 306 | 7-methyl-N-{5- [5- (trifluoromethyl)- 1H- benzimidazol-2- yl]bicyclo[3.1.1] hept-1-yl}-2,3- dihydro-1,4- benzodioxine- 6-carboxamide | 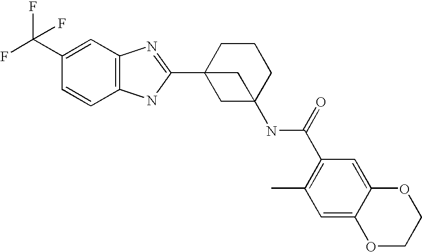 | HPLC Rt = 2.0 MS: [M + H] = 472.1 | 110.3 | 50 |
| 307 | N-[(1R,3S)-3- (1H- benzimidazol-2- yl)cyclohexyl]- 7-methyl-2,3- dihydro-1,4- benzodioxine- 6-carboxamide | 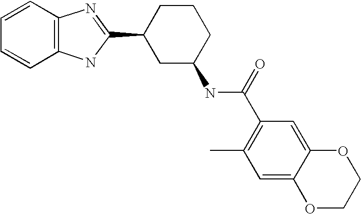 | HPLC Rt = 1.3 MS: [M + H] = 392.2 | 90.51 | 50 |
| 308 | N-[(1R,3S)-3- (1H- benzimidazol-2- yl)cyclohexyl]- 1,2,3,4- tetrahydroiso- quinoline-7- carboxamide HCl | 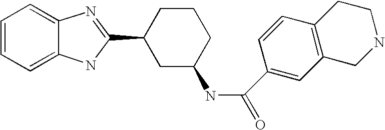 | HPLC Rt = 1.5 MS: [M + H] = 375.3 | 96.16 | 4 |
| 309 | N-[(1R,3S)-3- (1H- benzimidazol-2- yl)cyclohexyl]- 1,2,3,4- tetrahydroiso- quinoline-6- carboxamide HCl | 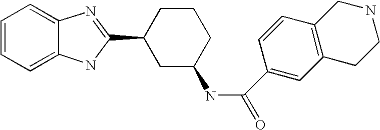 | HPLC Rt = 1.4 MS: [M + H] = 375.3 | 102.1 | 4 |
| 310 | N-[(1R,3S)-3- (1-methyl-1H- benzimidazol-2- yl)cyclohexyl]- 1,2,3,4- tetrahydroiso- quinoline-7- carboxamide HCl | 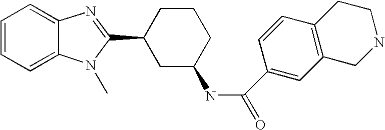 | HPLC Rt = 1.5 MS: [M + H] = 389.3 | 91.63 | 3, 4 |
| 311 | N-[(1R,3S)-3- (1-methyl-1H- benzimidazol-2- yl)cyclohexyl]- 1,2,3,4- tetrahydroiso- quinoline-6- carboxamide HCl | 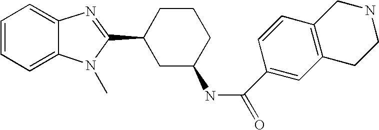 | HPLC Rt = 1.5 MS: [M + H] = 389.3 | 100.3 | 3, 4 |

1. A compound of formula I: 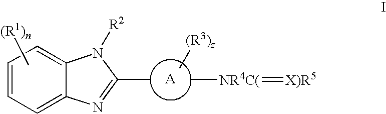
or a pharmaceutically acceptable salt wherein:
A is a 1,3-(C₃-C₁₂)cycloalkyl;
each R¹ is independently selected from the group consisting of halo, —(CH₂)ₜOH, —(CH₂)ₜCF₃, —(CH₂)ₜC≡N, —NO₂, —(CH₂)ₜN[(CH₂)ₜR⁹]₂, —(CH₂)ₜ(C═O)N[(CH₂)ₜR⁹], —(CH₂)ₜN[(CH₂)ₜR⁹](C═O)[(CH₂)ₜR⁹], —(CH₂)ₜN[(CH₂)ₜR⁹]S(O)_{w}[(CH₂)ₜR⁹], —(CH₂)ₜS(O)_{w}N[(CH₂)ₜR⁹]₂, —(CH₂)ₜS(O)_{w}[(CH₂)ₜR⁹], —(CH₂)ₜR⁹, —(CH₂)ₜO[(CH₂)ₜR⁹], —(CH₂)ₜ(C═O)[(CH₂)ₜR⁹], —(CH₂)ₜ(C═O)O[(CH₂)ₜR⁹], —(CH₂)ₜO(C═O)[(CH₂)ₜR⁹], —N[(CH₂)ₜR⁹](C═O)N[(CH₂)ₜR⁹]₂, —(CH₂)ₜ(C₃-C₁₂)carbocyclyl, —(CH₂)ₜ(C₆-C₁₀ aryl), and —(CH₂)ₜ(4 to 14 membered heterocyclyl) wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂;
R² is selected from the group consisting of hydrogen, —(C₁-C₆)alkyl, —(CH₂)_{q}OH, —(CH₂)_{q}O(C₁-C₆)alkyl, —(CH₂)_{q}O(C₁-C₆)alkylOH, —(CH₂)ₚCF₃, and —(CH₂)ₚCN;
each R³ is independently selected from the group consisting of hydrogen, —CN, halo, hydroxy, —(C₁-C₆)alkyl, —(C₂-C₆)alkenyl, —(C₂-C₆)alkynyl, —(C₁-C₆)alkoxy, —CF₃, —OCF₃, —N[(CH₂)ₜR⁹]₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, —(C₃-C₁₂)carbocyclyl, —(CH₂)ₜ(C₆-C₁₀ aryl), —(CH₂)ₜ(4 to 14 membered heterocyclyl), —(CH₂)ₜO(CH₂)ₜ(C₆-C₁₀ aryl), —(CH₂)ₜO(CH₂)ₜ(4 to 14 membered heterocyclyl), —(CH₂)ₜ(C═O)(CH₂)ₜ(C₆-C₁₀ aryl), —(CH₂)ₜ(C═O)(CH₂)ₜ(4 to 14 membered heterocyclyl), wherein said heterocyclyl has 1 to 4 ring heteroatoms selected from the group consisting of N, O, and S, and wherein each said alkyl, cycloalkyl, aryl, and heterocyclyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —(C═O)(C₁-C₆)alkyl, and —(C₃-C₁₂)carbocyclyl;
R⁴ is selected from the group consisting of hydrogen, —(C₁-C₆)alkyl, —(CH₂)_{q}OH, —(CH₂)_{q}O(C₁-C₆)alkyl, —(CH₂)_{q}O(C₁-C₆)alkylOH, —(CH₂)ₚCF₃, —(CH₂)ₚCN, —(CH₂)ₚNH₂, —(CH₂)ₚNH(C₁-C₆)alkyl, and —(CH₂)ₚN[(C₁-C₆)alkyl]₂;
R⁵ is selected from the group consisting of —(C₁-C₆)alkyl, —(C₂-C₆)alkenyl, —(C₂-C₆)alkynyl, —(CH₂)ₜ(C₃-C₁₂)carbocyclyl, —(CH₂)ₜ(C₆-C₁₀)aryl, —(CH₂)ₚ(C₁-C₆)alkoxy, —(CH₂)ₜO(CH₂)ₜ(C₆-C₁₀)aryl, —(CH₂)ₜN[(CH₂)ₜR⁹]₂, —(CH₂)ₜN[(CH₂)ₜR⁹](C₆-C₁₀)aryl, —(CH₂)ₜ(4 to 14 membered heterocyclyl), —(CH₂)ₜO(CH₂)ₜ(4 to 14 membered heterocyclyl) and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, and wherein each said (CH₂) moiety, alkyl, alkynyl, alkenyl, carbocyclyl, aryl, and heterocyclyl are independently optionally substituted by 1 to 5 substituents selected from R⁶;
each R⁶ is independently selected from the group consisting of azide, halo, —NO₂, —OR⁷, —(CH₂)ₜ(R⁷), —CF₃, —OCF₃, —OCHF₂, —OCH₂F, —O(CH₂)ₜ(C₆-C₁₀)aryl(R⁷), —(CH₂)ₜC≡N, —(C₁-C₆)alkyl, —(CH₂)ₜ(C₃-C₁₂)carbocyclyl(R⁷), —(CH₂)ₜ(C₆-C₁₀)aryl(R⁷), —(CH₂)ₜ(4 to 14 membered heterocyclyl)(R⁷), —(CH₂)ₜSR⁷, —(CH₂)ₜ(S═O)R⁷, —(CH₂)ₜS(═O)₂R⁷, —[C(R⁶)₂]ₜN(R⁷)S(═O)₂R⁷, —S(═O)₂N(R⁷)₂, —(C═O)R⁷, —(C═O)OR⁷, —[C(R⁷)₂]ₜO(C═O)R⁷, —[C(R⁷)₂]ₜO(C═O)N(R⁷)₂, —[C(R⁷)₂]ₜN(R⁷)(C═O)R⁷, —[C(R⁷)₂]ₜN(R⁷)₂, —8 C(R⁷)₂]ₜOR⁷, —[C(R⁷)₂]ₜN(R⁷)(C═O)OR⁷, —[C(R⁷)₂]ₜN(R⁷)(C═O)N(R⁷)₂, —[C(R⁷)₂]ₜN(R⁷)S(═O)₂N(R⁷)₂, —[C(R⁷)₂]ₜN(R⁷)N(R⁷)₂, —(C═O)N(R⁷)₂, —O(C═O)N(R⁷ )₂, —[C(R⁷)₂]ₜOR⁷, —C(R⁷)₂SR⁷, —[C(R⁷)₂]ₜ(S═O)R⁷, —[C(R⁷)₂]ₜS(═O)₂R⁷, —[C(R⁷)₂]ₜS(═O)₂N(R⁷)₂, —[C(R⁷)₂]ₜN(R⁷)(C═O)R⁷, —[C(R⁷)₂]ₜN(R⁷)(C═O)OR⁷, —C(R⁷)═NN(R⁷)₂, —C(R⁷)═NOR⁷, —C(R⁷)₂N(R⁷)N(R⁷)₂, —[C(R⁷)₂]ₜN(R⁷)S(═O)₂N(R⁷)₂, and —[C(R⁷)₂]ₜN(R⁷)(C═O)N(R⁷)₂;
each R⁷ is independently selected from H, —CF₃, —(C₁-C₆)alkyl, —(C₂-C₆)alkenyl, —(C₂-C₆)alkynyl, —(C₃-C₁₂)carbocyclyl, and —(C₆-C₁₀)aryl, or two R⁷ groups on the same nitrogen atom may be taken together with the nitrogen atom to form a 5 to 8 membered heterocyclyl ring, wherein said heterocyclyl ring has 1 to 3 ring heteroatoms selected from the group consisting of N, O, and S, or two R⁷ groups on the same carbon atom may be taken together with the carbon atom to form a 3 to 7 membered carbocyclyl ring and wherein each said alkyl, alkenyl, aryl, heterocyclyl and carbocyclyl may optionally be substituted by one to three substituents independently selected from the group consisting of halo, hydroxy, —CN, —(C₁-C₆)alkyl, —(C₁-C₆)alkoxy, —CF₃, —OCF₃, —N[(CH₂)ₜR⁹]₂, —NO₂, —S(C₁-C₆)alkyl, —(S═O)(C₁-C₆)alkyl, —S(═O)₂(C₁-C₆)alkyl, —(C═O)O(C₁-C₆)alkyl, —C(═O)(C₁-C₆)alkyl, and —(C₃-C₁₂)carbocyclyl; X is selected from the group consisting of O, S, and NR⁸;
R⁸ is selected from the group consisting of hydrogen, —(C₁-C₆)alkyl, —(CH₂)ₜC≡N, —NO₂, and —S(═O)₂R⁹;
each R⁹ is independently selected from the group consisting of H, —(C₁-C₆)alkyl, —(CH₂)ₜOH, —(CH₂)ₜ(C₆-C₁₀ aryl), —(CH₂)ₜ(C₃-C₁₂)carbocyclyl, and —(CH₂)ₜ(4 to 14 membered heterocyclyl), wherein said heterocyclyl has 1 to 3 ring heteroatoms selected from the group consisting of N, O, and S, or two R⁹ groups on the same nitrogen atom may be taken together with the nitrogen atom to form a 5 to 8 membered heterocyclyl ring wherein said heterocyclyl ring optionally has 1 to 3 ring additional heteroatoms selected from the group consisting of N, O, and S, or two R⁹ groups on the same carbon atom may be taken together with the carbon atom to form a 3 to 7 membered carbocyclyl ring, wherein each said alkyl, aryl, (CH₂) moiety, carbocyclyl, and heterocyclyl may optionally be substituted by one to three substituents independently selected from the group consisting of —(C₁-C₆)alkyl, halo, hydroxy, —(C₁-C₆)alkoxy, —CN, —(CH₂)ₜCF₃, —(CH₂)ₜ(C₆-C₁₀ aryl), —NH(C₁-C₆)alkyl, —N[(C₁-C₆)alkyl]₂ and —(CH₂)ₜ(4 to 14 membered heterocyclyl) wherein said heterocyclyl has 1 to 3 ring heteroatoms selected from the group consisting of N, O, and S;
each p is an integer independently selected from 1, 2, 3, 4, or 5;
each t is an integer independently selected from 0, 1, 2, 3, 4, or 5;
each n is an integer independently selected from 0, 1, 2, 3, or 4;
each q is an integer independently selected from 2, 3, 4, or 5;
each w is an integer independently selected from 0, 1, or 2; and
each z is an integer independently selected from 0, 1, 2, 3, 4, 5, 6, or 7.

2. A compound according to claim 1, or pharmaceutically acceptable salt thereof, wherein A is a 1,3-(C₃-C₉)cycloalkyl.

3. A compound according to claim 1, or pharmaceutically acceptable salt thereof, wherein A is a 1,3-cyclohexyl.

4. A compound according to claim 1, or pharmaceutically acceptable salt thereof, wherein A is a bicyclo[3.1.1]heptanyl, said ring may optionally contain 1 or 2 double bonds.

5. A compound according to claim 1, or pharmaceutically acceptable salt thereof, wherein each R¹ is independently selected from the group consisting of halo, —(CH₂)ₜCF₃, —(CH₂)ₜC≡N, —(CH₂)ₜR⁹, and —(CH₂)ₜO[(CH₂)ₜR⁹], wherein each said (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

6. A compound according to claim 1, or pharmaceutically acceptable salt thereof, wherein A is a 1,3-cyclohexyl and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

7. A compound according to claim 1, or pharmaceutically acceptable salt thereof, wherein A is a bicyclo[3.1.1]heptanyl, said ring may optionally contain 1 or 2 double bonds, and wherein R¹ is selected from the group consisting of halo and —(CH₂)ₜCF₃ wherein each (CH₂) moiety may optionally be substituted by one to two substituents independently selected from the group consisting of —(C₁-C₆)alkyl, halo, hydroxy, —(C₁-C₆)alkoxy, —CN, —(CH₂)ₜCF₃, and —N[(CH₂)ₜR⁹]₂.

8. A compound according to claim 1, or pharmaceutically acceptable salt thereof, wherein R² is hydrogen.

9. A compound according to claim 1, or pharmaceutically acceptable salt thereof, wherein A is a 1,3-cyclohexyl, and wherein R⁴ is selected from the group consisting of hydrogen and —(C₁-C₆)alkyl.

10. A compound according to claim 1, or pharmaceutically acceptable salt thereof, wherein A is a bicyclo[3.1.1]heptanyl, and wherein R⁴ is selected from the group consisting of hydrogen and —(C₁-C₆)alkyl.

11. A compound according to claim 1, or pharmaceutically acceptable salt thereof, wherein R⁵ is selected from the group consisting of —(CH₂)ₜ(C₃-C₁₂)carbocyclyl, —(CH₂)ₜ(C₆-C₁₀)aryl, —(CH₂)ₚ(C₁-C₆)alkoxy, —(CH₂)ₜO(CH₂)ₜ(C₆-C₁₀)aryl, —(CH₂)ₜN[(CH₂)ₜR⁹]₂, —(CH₂)ₜN[(CH₂)ₜR⁹]C₆-C₁₀)aryl, —(CH₂)ₜ(4 to 14 membered heterocyclyl), -(CH₂)ₜO(CH₂)t(4 to 14 membered heterocyclyl) and —(CH₂)ₜ(N[(CH₂)ₜR⁹])(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, wherein each said (CH₂) moiety may optionally be substituted by one to two substituents independently selected substituents selected from R⁶, and wherein each said carbocyclyl, aryl, and heterocyclyl are independently optionally substituted by 1 to 3 substituents selected from R⁶.

12. A compound according to claim 10, or pharmaceutically acceptable salt thereof, wherein R⁵ is —(CH₂)ₜ(C₆-C₁₀)aryl, wherein each said (CH₂) moiety may optionally be substituted by one to two substituents independently selected substituents selected from R⁶, and wherein each said aryl and heterocyclyl are independently optionally substituted by 1 to 3 substituents selected from R⁶.

13. A compound according to claim 10, or a pharmaceutically acceptable salt thereof, wherein R⁵ is —(CH₂)ₜ(4 to 14 membered heterocyclyl), wherein said heterocyclyl has 1 to 3 ring heteroatoms selected from the group consisting of N, O, and S, and wherein one or two carbon atoms of said heterocyclyl are optionally substituted with an oxo group, and wherein each said (CH₂) moiety may optionally be substituted by one to two substituents independently selected substituents selected from R⁶.

14. A compound according to claim 1, or pharmaceutically acceptable salt thereof, wherein X is O.

15. A compound according to claim 1 selected from the group consisting of:
N-((1R,3S)-3-(1H-benzo[d]imidazol-2-yl)cyclohexyl)-2,3-dihydro-[1,4]dioxino[2,3-c]pyridine-7-carboxamide,
1-[(1R,3S)-3-(1H-benzimidazol-2-yl)cyclohexyl]-3-[4-chloro-3-(trifluoromethyl)phenyl]urea,
N-{3-[6-(trifluoromethyl)-1H-benzimidazol-2-yl]cyclohexyl}-2,3-dihydro-1,4-benzodioxine-6-carboxamide,
N-{3-[6-(dimethylamino)-1H-benzimidazol-2-yl]cyclohexyl}-2,3-dihydro-1,4-benzodioxine-6-carboxamide,
N-((3S)-3-(1H-benzo[d]imidazol-2-yl)cyclohexyl)-2,3-dihydrobenzo[b][1,4]dioxine-6-carbothioamide,
N-[(1R,3S)-3-(5-chloro-1H-benzimidazol-2-yl)cyclohexyl]-1-methyl-2-oxo-1,2,3,4-tetrahydroquinoline-6-carboxamide,
4-methyl-3-oxo-N-{(1R,3S)-3-[5-(trifluoromethyl)-1H-benzimidazol-2-yl]cyclohexyl}-3,4-dihydro-2H-1,4-benzoxazine-6-carboxamide,
1-[(1R,3S)-3-(1H-benzimidazol-2-yl)cyclohexyl]-3-(3,5-dichlorophenyl)urea,
N-[(1R,3S)-3-(5-chloro-1H-benzimidazol-2-yl)cyclohexyl]-3,5-dimethoxybenzamide,
N-[(1R,3S)-3-(5-chloro-1H-benzimidazol-2-yl)cyclohexyl]-2-oxo-1,2,3,4-tetrahydroquinoline-6-carboxamide,
1-[(1R,3S)-3-(1H-benzimidazol-2-yl)cyclohexyl]-3-(3,4-dichlorophenyl)urea,
N-[3-(6-{[(2-methoxyethyl)amino]methyl}-1-methyl-1H-benzimidazol-2-yl)cyclohexyl]-2,3-dihydro-1,4-benzodioxine-6-carboxamide,
1,3-dimethyl-N-{(1R,3S)-3-[5-(trifluoromethyl)-1H-benzimidazol-2-yl]cyclohexyl}-1H-thieno[2,3-c]pyrazole-5-carboxamide,
2-oxo-N-{(1R,3S)-3-[5-(trifluoromethyl)-1H-benzimidazol-2-yl]cyclohexyl}-1,2,3,4-tetrahydroquinoline-6-carboxamide,
4-benzoyl-N-{5-[5-(trifluoromethyl)-1H-benzimidazol-2-yl]bicyclo[3.1.1]hept-1-yl}benzamide,
3,5-dimethoxy-N-{(1R,3S)-3-[6-(trifluoromethyl)-1H-benzimidazol-2-yl]cyclohexyl}benzamide,
1-methyl-2-oxo-N-{(1R,3S)-3-[5-(trifluoromethyl)-1H-benzimidazol-2-yl]cyclohexyl}-1,2,3,4-tetrahydroquinoline-6-carboxamide,
N-[3-(6-bromo-1H-benzimidazol-2-yl)cyclohexyl]-2,3-dihydro-1,4-benzodioxine-6-carboxamide,
3,5-dimethoxy-N-{5-[5-(trifluoromethyl)-1H-benzimidazol-2-yl]bicyclo[3.1.1]hept-1-yl}benzamide,
1-[(1R,3S)-3-(1H-benzimidazol-2-yl)cyclohexyl]-3-(4-isopropylphenyl)urea,
N-[(1R,3S)-3-(1-methyl-1H-benzimidazol-2-yl)cyclohexyl]-2,3-dihydro-1,4-benzodioxine-6-carboxamide,
N-{5-[5-(trifluoromethyl)-1H-benzimidazol-2-yl]bicyclo[3.1.1]hept-1-yl}-2,3-dihydro-1,4-benzodioxine-6-carboxamide,
N-((1S,3S)-3-(1H-benzo[d]imidazol-2-yl)cyclohexyl)-3-oxo-3,4-dihydro-2H-benzo[b][1,4]oxazine-6-carboxamide,
N-[3-(6-bromo-1-methyl-1H-benzimidazol-2-yl)cyclohexyl]-3,5-dimethoxybenzamide,
4-(trifluoroacetyl)-N-{5-[5-(trifluoromethyl)-1H-benzimidazol-2-yl]bicyclo[3.1.1]hept-1-yl}benzamide,
N-[(1R,3S)-3-(5-chloro-1H-benzimidazol-2-yl)cyclohexyl]-4-methyl-3-oxo-3,4-dihydro-2H-1,4-benzoxazine-6-carboxamide,
1-(4-cyanophenyl)-3-{5-[5-(trifluoromethyl)-1H-benzimidazol-2-yl]bicyclo[3.1.1]hept-1-yl}urea,
3,5-dimethoxy-N-{3-[6-(trifluoromethyl)-1H-benzimidazol-2-yl]cyclohexyl}benzamide,
N-[3-(1H-benzimidazol-2-yl)cyclohexyl]-2-(5-chloro-1H-benzimidazol-2-yl)acetamide,
N-{(3S)-3-[5-(trifluoromethyl)-1H-benzimidazol-2-yl]cyclohexyl}-2,3-dihydro-1,4-benzodioxine-6-carboxamide,
N-[(1R,3S)-3-(6-chloro-1H-benzimidazol-2-yl)cyclohexyl]-2,3-dihydro-1,4-benzodioxine-6-carboxamide,
1-[(1R,3S)-3-(1H-benzimidazol-2-yl)cyclohexyl]-3-[4-(trifluoromethyl)phenyl]urea,
N-[(1R,3S)-3-(1-methyl-1H-benzimidazol-2-yl)cyclohexyl]-2,3-dihydro-1,4-benzodioxine-6-carboxamide,
N-{3-[6-(methoxymethyl)-1-methyl-1H-benzimidazol-2-yl]cyclohexyl}-2,3-dihydro-1,4-benzodioxine-6-carboxamide,
3,5-dimethoxy-N-{(1R,3S)-3-[6-(trifluoromethyl)-1H-benzimidazol-2-yl]cyclohexyl}benzamide,
N-[3-(7-methyl-1H-benzimidazol-2-yl)cyclohexyl]-2,3-dihydro-1,4-benzodioxine-6-carboxamide,
N-[3-(6-tert-butyl-1H-benzimidazol-2-yl)cyclohexyl]-2,3-dihydro-1,4-benzodioxine-6-carboxamide,
N-[(3R)-3-(5-methoxy-1H-benzimidazol-2-yl)cyclohexyl]-2,3-dihydro-1,4-benzodioxine-6-carboxamide,
N-[3-(1H-benzimidazol-2-yl)cyclohexyl]-6-methoxy-1-methyl-1H-indole-2-carboxamide,
N-[3-(1H-benzimidazol-2-yl)cyclohexyl]-1H-indole-6-carboxamide,
3,5-dimethoxy-N-{(1R,3S)-3-[6-(trifluoromethyl)-1H-benzimidazol-2-yl]cyclohexyl}benzamide,
N-[(1R,3S)-3-(6-methyl-1H-benzimidazol-2-yl)cyclohexyl]-2,3-dihydro-1,4-benzodioxine-6-carboxamide,
N-[3-(6-chloro-1H-benzimidazol-2-yl)cyclohexyl]-2,3-dihydro-1,4-benzodioxine-6-carboxamide,
N-[3-(5,6-dimethyl-1H-benzimidazol-2-yl)cyclohexyl]-2,3-dihydro-1,4-benzodioxine-6-carboxamide,
1-[(1R,3S)-3-(1H-benzimidazol-2-yl)cyclohexyl]-3-(6-fluoro-4H-1,3-benzodioxin-8-yl)urea,
N-{3-[6-(cyanomethyl)-1-methyl-1H-benzimidazol-2-yl]cyclohexyl}-2,3-dihydro-1,4-benzodioxine-6-carboxamide,
N-[5-(5-chloro-1H-benzimidazol-2-yl)bicyclo[3.1.1]hept-1-yl]-3,5-dimethoxybenzamide,
N-[3-(1H-benzimidazol-2-yl)cyclohexyl]-2-oxo-1,2,3,4-tetrahydroquinoline-6-carboxamide,
N-[(3S)-3-(5-bromo-1H-benzimidazol-2-yl)cyclohexyl]-2,3-dihydro-1,4-benzodioxine-6-carboxamide, and
N-{(1R,3S)-3-[5-(trifluoromethyl)-1H-benzimidazol-2-yl]cyclohexyl}-4,5,6,7,8,9-hexahydro-1H-cycloocta[c]pyrazole-3-carboxamide, or pharmaceutically acceptable salt thereof.

16. A compound according to claim 1 selected from the group consisting of:
N-((1R,3S)-3-(1H-benzo[d]imidazol-2-yl)cyclohexyl)-2,3-dihydro-[1,4]dioxino[2,3-c]pyridine-7-carboxamide,
1-[(1R,3S)-3-(1H-benzimidazol-2-yl)cyclohexyl]-3-[4-chloro-3-(trifluoromethyl)phenyl]urea,
N-{3-[6-(trifluoromethyl)-1H-benzimidazol-2-yl]cyclohexyl}-2,3-dihydro-1,4-benzodioxine-6-carboxamide,
N-{3-[6-(dimethylamino)-1H-benzimidazol-2-yl]cyclohexyl}-2,3-dihydro-1,4-benzodioxine-6-carboxamide,
N-((3S)-3-(1H-benzo[d]imidazol-2-yl)cyclohexyl)-2,3-dihydrobenzo[b][1,4]dioxine-6-carbothioamide,
N-[(1R,3S)-3-(5-chloro-1H-benzimidazol-2-yl)cyclohexyl]-1-methyl-2-oxo-1,2,3,4-tetrahydroquinoline-6-carboxamide,
4-methyl-3-oxo-N-{(1R,3S)-3-[5-(trifluoromethyl)-1H-benzimidazol-2-yl]cyclohexyl}-3,4-dihydro-2H-1,4-benzoxazine-6-carboxamide,
1-[(1R,3S)-3-(1H-benzimidazol-2-yl)cyclohexyl]-3-(3,5-dichlorophenyl)urea,
N-[(1R,3S)-3-(5-chloro-1H-benzimidazol-2-yl)cyclohexyl]-3,5-dimethoxybenzamide,
N-[(1R,3S)-3-(5-chloro-1H-benzimidazol-2-yl)cyclohexyl]-2-oxo-1,2,3,4-tetrahydroquinoline-6-carboxamide,
1-[(1R,3S)-3-(1H-benzimidazol-2-yl)cyclohexyl]-3-(3,4-dichlorophenyl)urea,
N-[3-(6-{[(2-methoxyethyl)amino]methyl}-1-methyl-1H-benzimidazol-2-yl)cyclohexyl]-2,3-dihydro-1,4-benzodioxine-6-carboxamide,
1,3-dimethyl-N-{(1R,3S)-3-[5-(trifluoromethyl)-1H-benzimidazol-2-yl]cyclohexyl}-1H-thieno[2,3-c]pyrazole-5-carboxamide,
2-oxo-N-{(1R,3S)-3-[5-(trifluoromethyl)-1H-benzimidazol-2-yl]cyclohexyl}-1,2,3,4-tetrahydroquinoline-6-carboxamide,
4-benzoyl-N-{5-[5-(trifluoromethyl)-1H-benzimidazol-2-yl]bicyclo[3.1.1]hept-1-yl}benzamide,
3,5-dimethoxy-N-{(1R,3S)-3-[6-(trifluoromethyl)-1H-benzimidazol-2-yl]cyclohexyl}benzamide,
1-methyl-2-oxo-N-{(1R,3S)-3-[5-(trifluoromethyl)-1H-benzimidazol-2-yl]cyclohexyl}-1,2,3,4-tetrahydroquinoline-6-carboxamide,
N-[3-(6-bromo-1H-benzimidazol-2-yl)cyclohexyl]-2,3-dihydro-1,4-benzodioxine-6-carboxamide,
3,5-dimethoxy-N-{5-[5-(trifluoromethyl)-1H-benzimidazol-2-yl]bicyclo[3.1.1]hept-1-yl}benzamide,
1-[(1R,3S)-3-(1H-benzimidazol-2-yl)cyclohexyl]-3-(4-isopropylphenyl)urea,
N-[(1R,3S)-3-(1-methyl-1H-benzimidazol-2-yl)cyclohexyl]-2,3-dihydro-1,4-benzodioxine-6-carboxamide,
N-{5-[5-(trifluoromethyl)-1H-benzimidazol-2-yl]bicyclo[3.1.1]hept-1-yl}-2,3-dihydro-1,4-benzodioxine-6-carboxamide,
N-((1S,3S)-3-(1H-benzo[d]imidazol-2-yl)cyclohexyl)-3-oxo-3,4-dihydro-2H-benzo[b][1,4]oxazine-6-carboxamide,
N-[3-(6-bromo-1-methyl-1H-benzimidazol-2-yl)cyclohexyl]-3,5-dimethoxybenzamide, and
4-(trifluoroacetyl)-N-{5-[5-(trifluoromethyl)-1H-benzimidazol-2-yl]bicyclo[3.1.1]hept-1-yl}benzamide, or the pharmaceutically acceptable salt thereof.

17. A method for the treatment of abnormal cell growth in a mammal comprising administering to said mammal an amount of a compound according to claim 1, or pharmaceutically acceptable salt thereof, that is effective in treating abnormal cell growth.

18. A method according to claim 17, wherein said abnormal cell growth is cancer.

19. A method according to claim 18, wherein said cancer is selected from the group consisting of basal cell cancer, medulloblastoma cancer, liver cancer, rhabdomyosarcoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

20. A pharmaceutical composition comprising an amount of a compound of claim 1, or a pharmaceutically acceptable salt thereof, and a pharmaceutically acceptable carrier.
